(19) United States
(12) Patent Application Publication
(10) Pub. No.: US 2026/0271849 A1
(43) Pub. Date: Sep. 17, 2026

(54) METHODS FOR IMPROVING NUTRIENT CONTENT IN PLANT BASED ANIMAL FEEDSTOCKS

(71) Applicant: Biolumic Limited, Palmerston North (NZ)

(72) Inventor: Jason WARGENT, Palmerston North (NZ)

(21) Appl. No.: 19/669,615

(22) Filed: May 6, 2026

Related U.S. Application Data
(63) Continuation of application No. PCT/IB2024/061136, filed on Nov. 9, 2024.
(60) Provisional application No. 63/597,864, filed on Nov. 10, 2023.

(51) Int. Cl. A01H 3/02; A01C 1/00; A01G 22/22; A01G 22/40; A23K 10/30; B01D 53/85

(52) U.S. Cl. CPC A01H 3/02; A01C 1/00; A23K 10/30; B01D 53/85; A01G 22/22; A01G 22/40; B01D 2257/504; B01D 2258/06

(57) ABSTRACT

Provided are systems and methods for increasing nutrient content in plant based livestock feed sources through the administration of UV light to plant seed.

### CROSS-REFERENCE

This application is a continuation of International Patent Application No. PCT/IB2024/061136, filed Nov. 9, 2024, which claims the benefit of U.S. Provisional Patent Application No. 63/597,864 filed on Nov. 10, 2023, the disclosure of which is incorporated by reference in its entirety.

### BACKGROUND

Livestock nutrition is a critical factor impacting the quality and yield of the resulting animal products such as milk, meat, and wool. Feed plays a significant role in maintaining the health and productivity of livestock, primarily ruminants such as cattle, sheep, and goats. Among the various feeds used, grasses form an essential component of their diet, supplying a substantial proportion of the necessary nutrients for these animals.

Over time, traditional practices have involved using synthetic fertilizers to boost the nutritional quality of grasses. However, this approach has been linked with environmental and health concerns. The excessive use of synthetic fertilizers often leads to nutrient leaching into water bodies, causing pollution and biodiversity loss. Simultaneously, the accumulation of harmful residues in livestock products due to these fertilizers can pose risks to human health. Therefore, there exists a need for more sustainable and effective methods to increase the nutritional value of grasses used in livestock feed.

### SUMMARY

In certain aspects, disclosed herein is a method of increasing the performance of a plant, the method comprising: 1) administering UV-B light to a plant seed, 2) sowing the plant seed, 3) growing the plant from the plant seed, and 4) harvesting the plant, wherein the performance is measured by total lipid percentage, metabolizable energy, non-structural carbohydrates, crude fat concentration, forage biomass, tiller number, or shoot fresh weight. In some embodiments, the lipid percentage is increased in plants grown from plant seeds administered UV-B light prior to sowing as compared to plants grown from plant seeds not administered UV-B light prior to sowing. In some embodiments, the lipid percentage is increased by about 1% to about 100%, by about 1% to about 25%, or by about 5% to about 20%. In some embodiments, the lipid percentage is increased by about 1% to about 100% total plant lipids per unit of plant dry matter, or by about 1% to about 25% total plant lipids per unit of plant dry matter. In some embodiments, the lipid comprises a triacylglycerol, phospholipid, sterol, galactolipid, fatty acid, or tocopherol. In some embodiments, the phospholipid comprises phosphatidylcholine or phosphatidylethanolamine. In some embodiments, the sterol comprises sitosterol, campesterol, or stigmasterol. In some embodiments, the galactolipid comprises digalactosyldiacylglycerol or monogalactosyldiacylglycerol. In some embodiments, the tocopherol comprises Vitamin E. In some embodiments, the fatty acid comprises palmitic acid, palmitoleic acid, stearic acid, oleic acid, linoleic acid, linolenic acid, arachidic acid, gondoic acid, behenic acid, or erucic acid. In some embodiments, the total lipid percent is measured by gas chromatography or near infrared spectroscopy %. In some embodiments, the plant comprises a member of the Poaceae or Fabaceae family. In some embodiments, the member of the Poaceae family comprises rice, corn, or a grass. In some embodiments, the grass comprises a forage grass. In some embodiments, the grass comprises perennial ryegrass (Lolium perenne), timothy grass (Phleum pratense), tall fescue (Festuca arundinacea), kentucky bluegrass (Poa pratensis), bermuda grass (Cynodon dactylon), alfalfa grass (Medicago sativa), orchard grass (Dactylis glomerata), rye grass (Lolium), brome grass (Bromus), sudan grass (Sorghum×drummondii), bahia grass (Paspalum notatum), clover (trifolium), meadow fescue (Festuca pratensis), switchgrass (Panicum virgatum), prairie grass (Bromus kalmii), festulolium (hybrid of ryegrass and fescue), signal grass (Urochloa panicoides), Italian ryegrass (Lolium multiflorum), reed canary grass (Phalaris arundinacea), or millet (Panicum miliaceum or Setaria italica). In some embodiments, the grass comprises a ryegrass or a bluegrass. In some embodiments, the grass comprises ryegrass. In some embodiments, the ryegrass comprises annual ryegrass. In some embodiments, the member of the Fabaceae family comprises alfalfa, clover, soybean, peas, lentils, vetch, peanut, cowpea, chickpea or, lupins. In some embodiments, the member of the Fabaceae family comprises soybeans. In some embodiments, the UV-B light comprises UV-B light with a waveband of about 280-310 nm. In some embodiments, the UV-B light comprises UV-B light with a waveband of about 280-290 nm. In some embodiments, the UV-B light does not comprise UV-A or UV-C.

In certain aspects, disclosed herein is a method of increasing total lipid percentage in a plant, the method comprising: 1) administering UV-B light to a plant seed, 2) sowing the plant seed, 3) growing the plant from the plant seed, and 4) harvesting the plant. In certain aspects, disclosed herein is a method of increasing the performance of a plant, the method comprising: 1) administering UV-B light to a plant seed, 2) sowing the plant seed, 3) growing the plant from the plant seed, and 4) harvesting the plant, wherein the performance is measured by total lipid percentage, metabolizable energy, non-structural carbohydrates, crude fat concentration, forage biomass, tiller number, or shoot fresh weight. In some embodiments, the lipid percentage is increased in plants grown from plant seeds administered UV-B light prior to sowing as compared to plants grown from plant seeds not administered UV-B light prior to sowing. In some embodiments, the lipid percentage is increased by about 1% to about 100%, by about 1% to about 25%, or by about 5% to about 20%. In some embodiments, the lipid percentage is increased by about 1% to about 100% total plant lipids per unit of plant dry matter, or by about 1% to about 25% total plant lipids per unit of plant dry matter. In some embodiments, the lipid comprises a triacylglycerol, phospholipid, sterol, galactolipid, fatty acid, or tocopherol. In some embodiments, the phospholipid comprises phosphatidylcholine or phosphatidylethanolamine. In some embodiments, the sterol comprises sitosterol, campesterol, or stigmasterol. In some embodiments, the galactolipid comprises digalactosyldiacylglycerol or monogalactosyldiacylglycerol. In some embodiments, the tocopherol comprises Vitamin E. In some embodiments, the fatty acid comprises palmitic acid, palmitoleic acid, stearic acid, oleic acid, linoleic acid, linolenic acid, arachidic acid, gondoic acid, behenic acid, or erucic acid. In some embodiments, the total lipid percent is measured by gas chromatography or near infrared spectroscopy %. In some embodiments, the plant comprises a member of the Poaceae or Fabaceae family. In some embodiments, the member of the Poaceae family comprises rice, corn, or a grass. In some embodiments, the grass comprises a forage grass. In some embodiments, the grass comprises perennial ryegrass (Lolium perenne), timothy grass (Phleum pratense), tall fescue (Festuca arundinacea), kentucky bluegrass (Poa pratensis), bermuda grass (Cynodon dactylon), alfalfa grass (Medicago sativa), orchard grass (Dactylis glomerata), rye grass (lolium), brome grass (bromus), sudan grass (Sorghum×drummondii), bahia grass (Paspalum notatum), clover (trifolium), meadow fescue (Festuca pratensis), switchgrass (Panicum virgatum), prairie grass (Bromus kalmii), festulolium (hybrid of ryegrass and fescue), signal grass (Urochloa panicoides), Italian ryegrass (Lolium multiflorum), reed canary grass (Phalaris arundinacea), or millet (Panicum miliaceum or Setaria italica). In some embodiments, the grass comprises a ryegrass or a bluegrass. In some embodiments, the grass comprises ryegrass. In some embodiments, the ryegrass comprises annual ryegrass. In some embodiments, the member of the Fabaceae family comprises alfalfa, clover, soybean, peas, lentils, vetch, peanut, cowpea, chickpea or, lupins. In some embodiments, the member of the Fabaceae family comprises soybeans. In some embodiments, the UV-B light comprises UV-B light with a waveband of about 280-310 nm. In some embodiments, the UV-B light comprises UV-B light with a waveband of about 280-290 nm. In some embodiments, the UV-B light does not comprise UV-A or UV-C.

In certain aspects, disclosed herein is a method of increasing the total lipid percentage in a field for grazing, the method comprising: 1) administering UV-B light to a plurality of plant seeds, 2) sowing the plurality of plant seeds in the field, and 3) growing plants from the plurality of plant seeds. In certain aspects, disclosed herein is a method of increasing the performance of a plant, the method comprising: 1) administering UV-B light to a plant seed, 2) sowing the plant seed, 3) growing the plant from the plant seed, and 4) harvesting the plant, wherein the performance is measured by total lipid percentage, metabolizable energy, non-structural carbohydrates, crude fat concentration, forage biomass, tiller number, or shoot fresh weight. In some embodiments, the lipid percentage is increased in plants grown from plant seeds administered UV-B light prior to sowing as compared to plants grown from plant seeds not administered UV-B light prior to sowing. In some embodiments, the lipid percentage is increased by about 1% to about 100%, by about 1% to about 25%, or by about 5% to about 20%. In some embodiments, the lipid percentage is increased by about 1% to about 100% total plant lipids per unit of plant dry matter, or by about 1% to about 25% total plant lipids per unit of plant dry matter. In some embodiments, the lipid comprises a triacylglycerol, phospholipid, sterol, galactolipid, fatty acid, or tocopherol. In some embodiments, the phospholipid comprises phosphatidylcholine or phosphatidylethanolamine. In some embodiments, the sterol comprises sitosterol, campesterol, or stigmasterol. In some embodiments, the galactolipid comprises digalactosyldiacylglycerol or monogalactosyldiacylglycerol. In some embodiments, the tocopherol comprises Vitamin E. In some embodiments, the fatty acid comprises palmitic acid, palmitoleic acid, stearic acid, oleic acid, linoleic acid, linolenic acid, arachidic acid, gondoic acid, behenic acid, or erucic acid. In some embodiments, the total lipid percent is measured by gas chromatography or near infrared spectroscopy %. In some embodiments, the plant comprises a member of the Poaceae or Fabaceae family. In some embodiments, the member of the Poaceae family comprises rice, corn, or a grass. In some embodiments, the grass comprises a forage grass. In some embodiments, the grass comprises perennial ryegrass (Lolium perenne), timothy grass (Phleum pratense), tall fescue (Festuca arundinacea), kentucky bluegrass (Poa pratensis), bermuda grass (Cynodon dactylon), alfalfa grass (Medicago sativa), orchard grass (Dactylis glomerata), rye grass (lolium), brome grass (bromus), sudan grass (Sorghum×drummondii), bahia grass (Paspalum notatum), clover (trifolium), meadow fescue (Festuca pratensis), switchgrass (Panicum virgatum), prairie grass (Bromus kalmii), festulolium (hybrid of ryegrass and fescue), signal grass (Urochloa panicoides), Italian ryegrass (Lolium multiflorum), reed canary grass (Phalaris arundinacea), or millet (Panicum miliaceum or Setaria italica). In some embodiments, the grass comprises a ryegrass or a bluegrass. In some embodiments, the grass comprises ryegrass. In some embodiments, the ryegrass comprises annual ryegrass. In some embodiments, the member of the Fabaceae family comprises alfalfa, clover, soybean, peas, lentils, vetch, peanut, cowpea, chickpea or, lupins. In some embodiments, the member of the Fabaceae family comprises soybeans. In some embodiments, the UV-B light comprises UV-B light with a waveband of about 280-310 nm. In some embodiments, the UV-B light comprises UV-B light with a waveband of about 280-290 nm. In some embodiments, the UV-B light does not comprise UV-A or UV-C.

In certain aspects, disclosed herein is a method of increasing metabolizable energy in a plant, the method comprising: 1) administering UV-B light to a plant seed, 2) sowing the plant seed, 3) growing the plant from the plant seed, and 4) harvesting the plant. In some embodiments, metabolizable energy is increased in plants grown from plant seeds administered UV-B light prior to sowing as compared to plants grown from plant seeds not administered UV-B light prior to sowing. In some embodiments, metabolizable energy is increased by about 1% to about 100%, or by about 1% to about 15%. In some embodiments, metabolizable energy is increased by about 0.1 to about 10 MJ/Kg of dry matter, by about 0.1 to about 0.5 MJ/Kg of dry matter, or by about 1 to about 5 MJ/Kg of dry matter. %. In some embodiments, the plant comprises a member of the Poaceae or Fabaceae family. In some embodiments, the member of the Poaceae family comprises rice, corn, or a grass. In some embodiments, the grass comprises a forage grass. In some embodiments, the grass comprises perennial ryegrass (Lolium perenne), timothy grass (Phleum pratense), tall fescue (Festuca arundinacea), kentucky bluegrass (Poa pratensis), bermuda grass (Cynodon dactylon), alfalfa grass (Medicago sativa), orchard grass (Dactylis glomerata), rye grass (lolium), brome grass (bromus), sudan grass (Sorghum×drummondii), bahia grass (Paspalum notatum), clover (trifolium), meadow fescue (Festuca pratensis), switchgrass (Panicum virgatum), prairie grass (Bromus kalmii), festulolium (hybrid of ryegrass and fescue), signal grass (Urochloa panicoides), Italian ryegrass (Lolium multiflorum), reed canary grass (Phalaris arundinacea), or millet (Panicum miliaceum or Setaria italica). In some embodiments, the grass comprises a ryegrass or a bluegrass. In some embodiments, the grass comprises ryegrass. In some embodiments, the ryegrass comprises annual ryegrass. In some embodiments, the member of the Fabaceae family comprises alfalfa, clover, soybean, peas, lentils, vetch, peanut, cowpea, chickpea or, lupins. In some embodiments, the member of the Fabaceae family comprises soybeans. In some embodiments, the UV-B light comprises UV-B light with a waveband of about 280-310 nm. In some embodiments, the UV-B light comprises UV-B light with a waveband of about 280-290 nm. In some embodiments, the UV-B light does not comprise UV-A or UV-C.

In certain aspects, disclosed herein is a method of increasing non-structural carbohydrates in a plant, the method comprising: 1) administering UV-B light to a plant seed, 2) sowing the plant seed, 3) growing the plant from the plant seed, and 4) harvesting the plant. In some embodiments, non-structural carbohydrates are increased in plants grown from plant seeds administered UV-B light prior to sowing as compared to plants grown from plant seeds not administered UV-B light prior to sowing. In some embodiments, non-structural carbohydrates are increased by about 1% to about 100%, or by about 1% to about 15%. In some embodiments, non-structural carbohydrates are increased by about 1% to about 10% of plant dry matter percentage, or by about 1% to about 5% of plant dry matter percentage. %. In some embodiments, the plant comprises a member of the Poaceae or Fabaceae family. In some embodiments, the member of the Poaceae family comprises rice, corn, or a grass. In some embodiments, the grass comprises a forage grass. In some embodiments, the grass comprises perennial ryegrass (Lolium perenne), timothy grass (Phleum pratense), tall fescue (Festuca arundinacea), kentucky bluegrass (Poa pratensis), bermuda grass (Cynodon dactylon), alfalfa grass (Medicago sativa), orchard grass (Dactylis glomerata), rye grass (lolium), brome grass (bromus), sudan grass (Sorghum×drummondii), bahia grass (Paspalum notatum), clover (trifolium), meadow fescue (Festuca pratensis), switchgrass (Panicum virgatum), prairie grass (Bromus kalmii), festulolium (hybrid of ryegrass and fescue), signal grass (Urochloa panicoides), Italian ryegrass (Lolium multiflorum), reed canary grass (Phalaris arundinacea), or millet (Panicum miliaceum or Setaria italica). In some embodiments, the grass comprises a ryegrass or a bluegrass. In some embodiments, the grass comprises ryegrass. In some embodiments, the ryegrass comprises annual ryegrass. In some embodiments, the member of the Fabaceae family comprises alfalfa, clover, soybean, peas, lentils, vetch, peanut, cowpea, chickpea or, lupins. In some embodiments, the member of the Fabaceae family comprises soybeans. In some embodiments, the UV-B light comprises UV-B light with a waveband of about 280-310 nm. In some embodiments, the UV-B light comprises UV-B light with a waveband of about 280-290 nm. In some embodiments, the UV-B light does not comprise UV-A or UV-C.

In certain aspects, disclosed herein is a method of increasing crude fat concentration in a plant, the method comprising: 1) administering UV-B light to a plant seed, 2) sowing the plant seed, 3) growing the plant from the plant seed, and 4) harvesting the plant. In some embodiments, crude fat concentration is increased in plants grown from plant seeds administered UV-B light prior to sowing as compared to plants grown from plant seeds not administered UV-B light prior to sowing. In some embodiments, crude fat concentration is increased by about 1% to about 100%, or by about 1% to about 15%. In some embodiments, crude fat concentration is increased by about 1% to about 100% of plant dry matter percentage, or by about 1% to about 15% of plant dry matter percentage. %. In some embodiments, the plant comprises a member of the Poaceae or Fabaceae family. In some embodiments, the member of the Poaceae family comprises rice, corn, or a grass. In some embodiments, the grass comprises a forage grass. In some embodiments, the grass comprises perennial ryegrass (Lolium perenne), timothy grass (Phleum pratense), tall fescue (Festuca arundinacea), kentucky bluegrass (Poa pratensis), bermuda grass (Cynodon dactylon), alfalfa grass (Medicago sativa), orchard grass (Dactylis glomerata), rye grass (lolium), brome grass (bromus), sudan grass (Sorghum×drummondii), bahia grass (Paspalum notatum), clover (trifolium), meadow fescue (Festuca pratensis), switchgrass (Panicum virgatum), prairie grass (Bromus kalmii), festulolium (hybrid of ryegrass and fescue), signal grass (Urochloa panicoides), Italian ryegrass (Lolium multiflorum), reed canary grass (Phalaris arundinacea), or millet (Panicum miliaceum or Setaria italica). In some embodiments, the grass comprises a ryegrass or a bluegrass. In some embodiments, the grass comprises ryegrass. In some embodiments, the ryegrass comprises annual ryegrass. In some embodiments, the member of the Fabaceae family comprises alfalfa, clover, soybean, peas, lentils, vetch, peanut, cowpea, chickpea or, lupins. In some embodiments, the member of the Fabaceae family comprises soybeans. In some embodiments, the UV-B light comprises UV-B light with a waveband of about 280-310 nm. In some embodiments, the UV-B light comprises UV-B light with a waveband of about 280-290 nm. In some embodiments, the UV-B light does not comprise UV-A or UV-C.

In certain aspects, disclosed herein is a method of increasing forage biomass in a grass, the method comprising: 1) administering UV-B light to a plant seed, 2) sowing the plant seed, 3) growing the plant from the plant seed, and 4) harvesting the plant. In some embodiments, forage biomass is increased in plants grown from plant seeds administered UV-B light prior to sowing as compared to plants grown from plant seeds not administered UV-B light prior to sowing. In some embodiments, forage biomass is increased by about 1% to about 100%, or by about 1% to about 15%. %. In some embodiments, the plant comprises a member of the Poaceae or Fabaceae family. In some embodiments, the member of the Poaceae family comprises rice, corn, or a grass. In some embodiments, the grass comprises a forage grass. In some embodiments, the grass comprises perennial ryegrass (Lolium perenne), timothy grass (Phleum pratense), tall fescue (Festuca arundinacea), kentucky bluegrass (Poa pratensis), bermuda grass (Cynodon dactylon), alfalfa grass (Medicago sativa), orchard grass (Dactylis glomerata), rye grass (lolium), brome grass (bromus), sudan grass (Sorghum×drummondii), bahia grass (Paspalum notatum), clover (trifolium), meadow fescue (Festuca pratensis), switchgrass (Panicum virgatum), prairie grass (Bromus kalmii), festulolium (hybrid of ryegrass and fescue), signal grass (Urochloa panicoides), Italian ryegrass (Lolium multiflorum), reed canary grass (Phalaris arundinacea), or millet (Panicum miliaceum or Setaria italica). In some embodiments, the grass comprises a ryegrass or a bluegrass. In some embodiments, the grass comprises ryegrass. In some embodiments, the ryegrass comprises annual ryegrass. In some embodiments, the member of the Fabaceae family comprises alfalfa, clover, soybean, peas, lentils, vetch, peanut, cowpea, chickpea or, lupins. In some embodiments, the member of the Fabaceae family comprises soybeans. In some embodiments, the UV-B light comprises UV-B light with a waveband of about 280-310 nm. In some embodiments, the UV-B light comprises UV-B light with a waveband of about 280-290 nm. In some embodiments, the UV-B light does not comprise UV-A or UV-C.

In certain aspects, disclosed herein is a method of increasing tiller number in a grass, the method comprising: 1) administering UV-B light to a plant seed, 2) sowing the plant seed, 3) growing the plant from the plant seed, and 4) harvesting the plant. In some embodiments, tiller number of the grass is increased in plants grown from plant seeds administered UV-B light prior to sowing as compared to plants grown from plant seeds not administered UV-B light prior to sowing. In some embodiments, tiller number is increased by about 1% to about 100%, or by about 1% to about 15%. %. In some embodiments, the plant comprises a member of the Poaceae or Fabaceae family. In some embodiments, the member of the Poaceae family comprises rice, corn, or a grass. In some embodiments, the grass comprises a forage grass. In some embodiments, the grass comprises perennial ryegrass (Lolium perenne), timothy grass (Phleum pratense), tall fescue (Festuca arundinacea), kentucky bluegrass (Poa pratensis), bermuda grass (Cynodon dactylon), alfalfa grass (Medicago sativa), orchard grass (Dactylis glomerata), rye grass (lolium), brome grass (bromus), sudan grass (Sorghum×drummondii), bahia grass (Paspalum notatum), clover (trifolium), meadow fescue (Festuca pratensis), switchgrass (Panicum virgatum), prairie grass (Bromus kalmii), festulolium (hybrid of ryegrass and fescue), signal grass (Urochloa panicoides), Italian ryegrass (Lolium multiflorum), reed canary grass (Phalaris arundinacea), or millet (Panicum miliaceum or Setaria italica). In some embodiments, the grass comprises a ryegrass or a bluegrass. In some embodiments, the grass comprises ryegrass. In some embodiments, the ryegrass comprises annual ryegrass. In some embodiments, the member of the Fabaceae family comprises alfalfa, clover, soybean, peas, lentils, vetch, peanut, cowpea, chickpea or, lupins. In some embodiments, the member of the Fabaceae family comprises soybeans. In some embodiments, the UV-B light comprises UV-B light with a waveband of about 280-310 nm. In some embodiments, the UV-B light comprises UV-B light with a waveband of about 280-290 nm. In some embodiments, the UV-B light does not comprise UV-A or UV-C.

In certain aspects, disclosed herein is a method of increasing fresh weight in a grass, the method comprising: 1) administering UV-B light to a plant seed, 2) sowing the plant seed, 3) growing the plant from the plant seed, and 4) harvesting the plant. In some embodiments, shoot fresh weight of the grass is increased in plants grown from plant seeds administered UV-B light prior to sowing as compared to plants grown from plant seeds not administered UV-B light prior to sowing. In some embodiments, shoot fresh weight of the grass increased by about 1% to about 100%, or by about 1% to about 15%. In some embodiments, shoot fresh weight of the grass increased by about 1 gram to about 100 grams, or by about 1 gram to about 10 grams. %. In some embodiments, the plant comprises a member of the Poaceae or Fabaceae family. In some embodiments, the member of the Poaceae family comprises rice, corn, or a grass. In some embodiments, the grass comprises a forage grass. In some embodiments, the grass comprises perennial ryegrass (Lolium perenne), timothy grass (Phleum pratense), tall fescue (Festuca arundinacea), kentucky bluegrass (Poa pratensis), bermuda grass (Cynodon dactylon), alfalfa grass (Medicago sativa), orchard grass (Dactylis glomerata), rye grass (lolium), brome grass (bromus), sudan grass (Sorghum×drummondii), bahia grass (Paspalum notatum), clover (trifolium), meadow fescue (Festuca pratensis), switchgrass (Panicum virgatum), prairie grass (Bromus kalmii), festulolium (hybrid of ryegrass and fescue), signal grass (Urochloa panicoides), Italian ryegrass (Lolium multiflorum), reed canary grass (Phalaris arundinacea), or millet (Panicum miliaceum or Setaria italica). In some embodiments, the grass comprises a ryegrass or a bluegrass. In some embodiments, the grass comprises ryegrass. In some embodiments, the ryegrass comprises annual ryegrass. In some embodiments, the member of the Fabaceae family comprises alfalfa, clover, soybean, peas, lentils, vetch, peanut, cowpea, chickpea or, lupins. In some embodiments, the member of the Fabaceae family comprises soybeans. In some embodiments, the UV-B light comprises UV-B light with a waveband of about 280-310 nm. In some embodiments, the UV-B light comprises UV-B light with a waveband of about 280-290 nm. In some embodiments, the UV-B light does not comprise UV-A or UV-C.

In certain aspects, disclosed herein is a method of increasing the total energy content per unit of feed consumed by an animal, the method comprising 1) administering UV-B light to a plurality of plant seeds, 2) sowing the plurality of plant seeds in a field, 3) growing plant from the plurality of plant seeds, and 4) feeding the animals the feed. In some embodiments, the total energy content per unit of feed consumed is increased by about 1% to about 100%, by about 1% to about 25%, or by about 5% to about 20%. In some embodiments, the animal is a cow or a sheep. %. In some embodiments, the plant comprises a member of the Poaceae or Fabaceae family. In some embodiments, the member of the Poaceae family comprises rice, corn, or a grass. In some embodiments, the grass comprises a forage grass. In some embodiments, the grass comprises perennial ryegrass (Lolium perenne), timothy grass (Phleum pratense), tall fescue (Festuca arundinacea), kentucky bluegrass (Poa pratensis), bermuda grass (Cynodon dactylon), alfalfa grass (Medicago sativa), orchard grass (Dactylis glomerata), rye grass (lolium), brome grass (bromus), sudan grass (Sorghum×drummondii), bahia grass (Paspalum notatum), clover (trifolium), meadow fescue (Festuca pratensis), switchgrass (Panicum virgatum), prairie grass (Bromus kalmii), festulolium (hybrid of ryegrass and fescue), signal grass (Urochloa panicoides), Italian ryegrass (Lolium multiflorum), reed canary grass (Phalaris arundinacea), or millet (Panicum miliaceum or Setaria italica). In some embodiments, the grass comprises a ryegrass or a bluegrass. In some embodiments, the grass comprises ryegrass. In some embodiments, the ryegrass comprises annual ryegrass. In some embodiments, the member of the Fabaceae family comprises alfalfa, clover, soybean, peas, lentils, vetch, peanut, cowpea, chickpea or, lupins. In some embodiments, the member of the Fabaceae family comprises soybeans. In some embodiments, the UV-B light comprises UV-B light with a waveband of about 280-310 nm. In some embodiments, the UV-B light comprises UV-B light with a waveband of about 280-290 nm. In some embodiments, the UV-B light does not comprise UV-A or UV-C.

In certain aspects, disclosed herein is a method of decreasing an area of field required for feeding animals, the method comprising: 1) administering UV-B light to a plant seed, 2) sowing the plant seed, 3) growing plant from the plant seed, and 4) grazing the animals on the field. In some embodiments, the area of the field required for feeding animals is decreased compared to a field wherein the plant seed was not administered UV-B light prior to sowing. In some embodiments, the area is decreased by about 1% to about 100%, or by about 1% to about 15%. In some embodiments, the area is decreased by about 0.1 to about 1000 acres. In some embodiments, the area is decreased by about 0.1 to about 1000 hectares. In some embodiments, the animal is a cow or a sheep. %. In some embodiments, the plant comprises a member of the Poaceae or Fabaceae family. In some embodiments, the member of the Poaceae family comprises rice, corn, or a grass. In some embodiments, the grass comprises a forage grass. In some embodiments, the grass comprises perennial ryegrass (Lolium perenne), timothy grass (Phleum pratense), tall fescue (Festuca arundinacea), kentucky bluegrass (Poa pratensis), bermuda grass (Cynodon dactylon), alfalfa grass (Medicago sativa), orchard grass (Dactylis glomerata), rye grass (lolium), brome grass (bromus), sudan grass (Sorghum×drummondii), bahia grass (Paspalum notatum), clover (trifolium), meadow fescue (Festuca pratensis), switchgrass (Panicum virgatum), prairie grass (Bromus kalmii), festulolium (hybrid of ryegrass and fescue), signal grass (Urochloa panicoides), Italian ryegrass (Lolium multiflorum), reed canary grass (Phalaris arundinacea), or millet (Panicum miliaceum or Setaria italica). In some embodiments, the grass comprises a ryegrass or a bluegrass. In some embodiments, the grass comprises ryegrass. In some embodiments, the ryegrass comprises annual ryegrass. In some embodiments, the member of the Fabaceae family comprises alfalfa, clover, soybean, peas, lentils, vetch, peanut, cowpea, chickpea or, lupins. In some embodiments, the member of the Fabaceae family comprises soybeans. In some embodiments, the UV-B light comprises UV-B light with a waveband of about 280-310 nm. In some embodiments, the UV-B light comprises UV-B light with a waveband of about 280-290 nm. In some embodiments, the UV-B light does not comprise UV-A or UV-C.

In certain aspects, disclosed herein is a method of reducing the methane production of animals, the method comprising 1) administering UV-B light to a plurality of plant seeds, 2) sowing the plurality of plant seeds in a field, 3) growing plants from the plant seeds, and 4) grazing the animals on the field or feeding the plants to the animals. In some embodiments, methane production is decreased in animals fed plants grown from plant seeds administered UV-B light prior to sowing as compared to plants grown from plant seeds not administered UV-B light prior to sowing. In some embodiments, methane production is decreased by about 1% to about 100%, or by about 1% to about 15%. In some embodiments, the animal is a cow or a sheep. %. In some embodiments, the plant comprises a member of the Poaceae or Fabaceae family. In some embodiments, the member of the Poaceae family comprises rice, corn, or a grass. In some embodiments, the grass comprises a forage grass. In some embodiments, the grass comprises perennial ryegrass (Lolium perenne), timothy grass (Phleum pratense), tall fescue (Festuca arundinacea), kentucky bluegrass (Poa pratensis), bermuda grass (Cynodon dactylon), alfalfa grass (Medicago sativa), orchard grass (Dactylis glomerata), rye grass (lolium), brome grass (bromus), sudan grass (Sorghum×drummondii), bahia grass (Paspalum notatum), clover (trifolium), meadow fescue (Festuca pratensis), switchgrass (Panicum virgatum), prairie grass (Bromus kalmii), festulolium (hybrid of ryegrass and fescue), signal grass (Urochloa panicoides), Italian ryegrass (Lolium multiflorum), reed canary grass (Phalaris arundinacea), or millet (Panicum miliaceum or Setaria italica). In some embodiments, the grass comprises a ryegrass or a bluegrass. In some embodiments, the grass comprises ryegrass. In some embodiments, the ryegrass comprises annual ryegrass. In some embodiments, the member of the Fabaceae family comprises alfalfa, clover, soybean, peas, lentils, vetch, peanut, cowpea, chickpea or, lupins. In some embodiments, the member of the Fabaceae family comprises soybeans. In some embodiments, the UV-B light comprises UV-B light with a waveband of about 280-310 nm. In some embodiments, the UV-B light comprises UV-B light with a waveband of about 280-290 nm. In some embodiments, the UV-B light does not comprise UV-A or UV-C.

In certain aspects, disclosed herein is a method of sequestering carbon from the atmosphere, the method comprising: 1) administering UV-B light to a plant seed, 2) sowing the plant seed, and 3) growing the plant from the plant seed. In some embodiments, sequestered carbon is increased in plants grown from plant seeds administered UV-B light prior to sowing as compared to plants grown from plant seeds not administered UV-B light prior to sowing. In some embodiments, sequestered carbon is increased by about 1% to about 100%, or by about 1% to about 15%. In some embodiments, the plant comprises a member of the Poaceae or Fabaceae family. In some embodiments, the member of the Poaceae family comprises rice, corn, or a grass. In some embodiments, the grass comprises a forage grass. In some embodiments, the grass comprises perennial ryegrass (Lolium perenne), timothy grass (Phleum pratense), tall fescue (Festuca arundinacea), kentucky bluegrass (Poa pratensis), bermuda grass (Cynodon dactylon), alfalfa grass (Medicago sativa), orchard grass (Dactylis glomerata), rye grass (lolium), brome grass (bromus), sudan grass (Sorghum×drummondii), bahia grass (Paspalum notatum), clover (trifolium), meadow fescue (Festuca pratensis), switchgrass (Panicum virgatum), prairie grass (Bromus kalmii), festulolium (hybrid of ryegrass and fescue), signal grass (Urochloa panicoides), Italian ryegrass (Lolium multiflorum), reed canary grass (Phalaris arundinacea), or millet (Panicum miliaceum or Setaria italica). In some embodiments, the grass comprises a ryegrass or a bluegrass. In some embodiments, the grass comprises ryegrass. In some embodiments, the ryegrass comprises annual ryegrass. In some embodiments, the member of the Fabaceae family comprises alfalfa, clover, soybean, peas, lentils, vetch, peanut, cowpea, chickpea or, lupins. In some embodiments, the member of the Fabaceae family comprises soybeans. In some embodiments, the UV-B light comprises UV-B light with a waveband of about 280-310 nm. In some embodiments, the UV-B light comprises UV-B light with a waveband of about 280-290 nm. In some embodiments, the UV-B light does not comprise UV-A or UV-C.

In certain aspects, disclosed herein is a plant generated a method as disclosed herein, wherein the plant comprises increased expression of an Oleosin (OLE), or diacylglycerol acyltransferase (DGAT) gene compared to a plant not generated by a method as disclosed herein. In some embodiments, the plant comprises an increased concentration of at least one lipid. In some embodiments, the expression of OLE or DGAT is not transgenically increased. In some embodiments, lipid concentration is increased by about 1% to about 100%, or by about 1% to about 15%. In some embodiments, lipid concentration is increased by about 0.1% to about 100% per unit of plant dry matter compared to a plant that is grown from a seed not administered UV-B light, or by about 0.1% to about 1% per unit of plant dry matter compared to a plant that is grown from a seed not administered UV-B light. In some embodiments, the plant comprises a member of the Poaceae or Fabaceae family. In some embodiments, the member of the Poaceae family comprises rice, corn, or a grass. In some embodiments, the grass comprises a forage grass. In some embodiments, the grass comprises perennial ryegrass (Lolium perenne), timothy grass (Phleum pratense), tall fescue (Festuca arundinacea), kentucky bluegrass (Poa pratensis), bermuda grass (Cynodon dactylon), alfalfa grass (Medicago sativa), orchard grass (Dactylis glomerata), rye grass (lolium), brome grass (bromus), sudan grass (Sorghum×drummondii), bahia grass (Paspalum notatum), clover (trifolium), meadow fescue (Festuca pratensis), switchgrass (Panicum virgatum), prairie grass (Bromus kalmii), festulolium (hybrid of ryegrass and fescue), signal grass (Urochloa panicoides), Italian ryegrass (Lolium multiflorum), reed canary grass (Phalaris arundinacea), or millet (Panicum miliaceum or Setaria italica). In some embodiments, the grass comprises a ryegrass or a bluegrass. In some embodiments, the grass comprises ryegrass. In some embodiments, the ryegrass comprises annual ryegrass. In some embodiments, the member of the Fabaceae family comprises alfalfa, clover, soybean, peas, lentils, vetch, peanut, cowpea, chickpea or, lupins. In some embodiments, the member of the Fabaceae family comprises soybeans. In some embodiments, the UV-B light comprises UV-B light with a waveband of about 280-310 nm. In some embodiments, the UV-B light comprises UV-B light with a waveband of about 280-290 nm. In some embodiments, the UV-B light does not comprise UV-A or UV-C. In some embodiments, the lipid comprises a triacylglycerol, phospholipid, sterol, galactolipid, fatty acid, or tocopherol. In some embodiments, the phospholipid comprises phosphatidylcholine or phosphatidylethanolamine. In some embodiments, the sterol comprises sitosterol, campesterol, or stigmasterol. In some embodiments, the galactolipid comprises digalactosyldiacylglycerol or monogalactosyldiacylglycerol. In some embodiments, the tocopherol comprises Vitamin E. In some embodiments, the fatty acid comprises palmitic acid, palmitoleic acid, stearic acid, oleic acid, linoleic acid, linolenic acid, arachidic acid, gondoic acid, behenic acid, or erucic acid. In some embodiments, the total lipid percent is measured by gas chromatography or near infrared spectroscopy %.

In certain aspects, disclosed herein is a plant comprising an increased concentration of at least one lipid, wherein the plant does not comprise a transgenic gene. In some embodiments, the transgenic gene is Oleosin (OLE) or diacylglycerol acyltransferase (DGAT). In some embodiments, the plant is grown from a plant seed that has been administered UV-B light. In some embodiments, lipid concentration is increased by about 1% to about 100%, or by about 1% to about 15%. In some embodiments, lipid concentration is increased by about 0.1% to about 100% per unit of plant dry matter compared to a plant that is grown from a seed not administered UV-B light, or by about 0.1% to about 1% per unit of plant dry matter compared to a plant that is grown from a seed not administered UV-B light. In some embodiments, the plant comprises a member of the Poaceae or Fabaceae family. In some embodiments, the member of the Poaceae family comprises rice, corn, or a grass. In some embodiments, the grass comprises a forage grass. In some embodiments, the grass comprises perennial ryegrass (Lolium perenne), timothy grass (Phleum pratense), tall fescue (Festuca arundinacea), kentucky bluegrass (Poa pratensis), bermuda grass (Cynodon dactylon), alfalfa grass (Medicago sativa), orchard grass (Dactylis glomerata), rye grass (lolium), brome grass (bromus), sudan grass (Sorghum×drummondii), bahia grass (Paspalum notatum), clover (trifolium), meadow fescue (Festuca pratensis), switchgrass (Panicum virgatum), prairie grass (Bromus kalmii), festulolium (hybrid of ryegrass and fescue), signal grass (Urochloa panicoides), Italian ryegrass (Lolium multiflorum), reed canary grass (Phalaris arundinacea), or millet (Panicum miliaceum or Setaria italica). In some embodiments, the grass comprises a ryegrass or a bluegrass. In some embodiments, the grass comprises ryegrass. In some embodiments, the ryegrass comprises annual ryegrass. In some embodiments, the member of the Fabaceae family comprises alfalfa, clover, soybean, peas, lentils, vetch, peanut, cowpea, chickpea or, lupins. In some embodiments, the member of the Fabaceae family comprises soybeans. In some embodiments, the UV-B light comprises UV-B light with a waveband of about 280-310 nm. In some embodiments, the UV-B light comprises UV-B light with a waveband of about 280-290 nm. In some embodiments, the UV-B light does not comprise UV-A or UV-C. In some embodiments, the lipid comprises a triacylglycerol, phospholipid, sterol, galactolipid, fatty acid, or tocopherol. In some embodiments, the phospholipid comprises phosphatidylcholine or phosphatidylethanolamine. In some embodiments, the sterol comprises sitosterol, campesterol, or stigmasterol. In some embodiments, the galactolipid comprises digalactosyldiacylglycerol or monogalactosyldiacylglycerol. In some embodiments, the tocopherol comprises Vitamin E. In some embodiments, the fatty acid comprises palmitic acid, palmitoleic acid, stearic acid, oleic acid, linoleic acid, linolenic acid, arachidic acid, gondoic acid, behenic acid, or erucic acid. In some embodiments, the total lipid percent is measured by gas chromatography or near infrared spectroscopy %.

In certain aspects, disclosed herein is a plant comprising an increased concentration of at least one lipid, wherein the plant comprises increased expression of an Oleosin (OLE) or diacylglycerol acyltransferase (DGAT) gene. In some embodiments, the expression of OLE or DGAT is not transgenically increased. In some embodiments, the plant is grown from a plant seed that has been administered UV-B light. In some embodiments, lipid concentration is increased by about 1% to about 100%, or by about 1% to about 15%. In some embodiments, lipid concentration is increased by about 0.1% to about 100% per unit of plant dry matter compared to a plant that is grown from a seed not administered UV-B light, or by about 0.1% to about 1% per unit of plant dry matter compared to a plant that is grown from a seed not administered UV-B light. In some embodiments, the plant comprises a member of the Poaceae or Fabaceae family. In some embodiments, the member of the Poaceae family comprises rice, corn, or a grass. In some embodiments, the grass comprises a forage grass. In some embodiments, the grass comprises perennial ryegrass (Lolium perenne), timothy grass (Phleum pratense), tall fescue (Festuca arundinacea), kentucky bluegrass (Poa pratensis), bermuda grass (Cynodon dactylon), alfalfa grass (Medicago sativa), orchard grass (Dactylis glomerata), rye grass (lolium), brome grass (bromus), sudan grass (Sorghum×drummondii), bahia grass (paspalum notatum), clover (trifolium), meadow fescue (Festuca pratensis), switchgrass (Panicum virgatum), prairie grass (Bromus kalmii), festulolium (hybrid of ryegrass and fescue), signal grass (Urochloa panicoides), Italian ryegrass (Lolium multiflorum), reed canary grass (Phalaris arundinacea), or millet (Panicum miliaceum or Setaria italica). In some embodiments, the grass comprises a ryegrass or a bluegrass. In some embodiments, the grass comprises ryegrass. In some embodiments, the ryegrass comprises annual ryegrass. In some embodiments, the member of the Fabaceae family comprises alfalfa, clover, soybean, peas, lentils, vetch, peanut, cowpea, chickpea or, lupins. In some embodiments, the member of the Fabaceae family comprises soybeans. In some embodiments, the UV-B light comprises UV-B light with a waveband of about 280-310 nm. In some embodiments, the UV-B light comprises UV-B light with a waveband of about 280-290 nm. In some embodiments, the UV-B light does not comprise UV-A or UV-C. In some embodiments, the lipid comprises a triacylglycerol, phospholipid, sterol, galactolipid, fatty acid, or tocopherol. In some embodiments, the phospholipid comprises phosphatidylcholine or phosphatidylethanolamine. In some embodiments, the sterol comprises sitosterol, campesterol, or stigmasterol. In some embodiments, the galactolipid comprises digalactosyldiacylglycerol or monogalactosyldiacylglycerol. In some embodiments, the tocopherol comprises Vitamin E. In some embodiments, the fatty acid comprises palmitic acid, palmitoleic acid, stearic acid, oleic acid, linoleic acid, linolenic acid, arachidic acid, gondoic acid, behenic acid, or erucic acid. In some embodiments, the total lipid percent is measured by gas chromatography or near infrared spectroscopy %.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts the crude fat percent of dry matter for plants grown from control ryegrass seeds and ryegrass seeds treated with UV-B.

FIG. 2 depicts the shoot fresh weight for plants grown from control ryegrass seeds and ryegrass seeds treated with UV-B.

FIG. 3 depicts the tiller number for plants grown from control ryegrass seeds and ryegrass seeds treated with UV-B.

FIG. 4 depicts metabolizable energy in corn sileage for plants grown from corn seeds treated with UV-B.

FIG. 5 depicts crude fat concentration in corn sileage for plants grown from corn seeds treated with UV-B.

### DETAILED DESCRIPTION

The present disclosure offers a novel method for increasing the energy content and metabolic utilization of crops utilizing ultraviolet (UV) light administration to plant seeds. The methods disclosed herein utilize the application of UV light to the seeds of a plant to stimulate plant growth, thereby leading to enhanced energy content and metabolic utilization of crops. Remarkably, the administration of UV light treatment to plant seeds is shown to increase lipid content, total metabolizable energy, and non-structural carbohydrates in plants grown from treated seeds. Increased lipid content is also directly related to reductions in methane production by grazing animals, as animals with high-fat diets have lower methane emissions. Additionally, the increased energy content in plants grown from UV treated seeds presents an opportunity to decrease the total amount of land needed to effectively feed grazing animals. Collectively, these improvements represent the ability of UV light administration to improve plant performance and grazing efficiency while simultaneously decreasing greenhouse gas emissions.

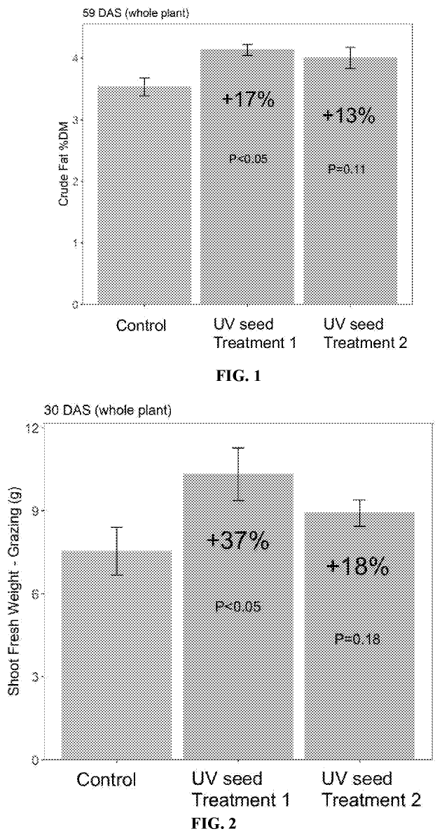

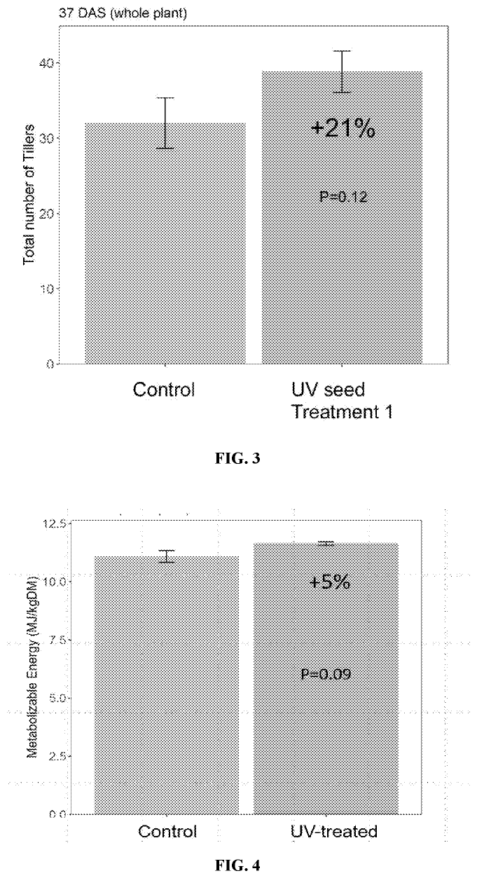

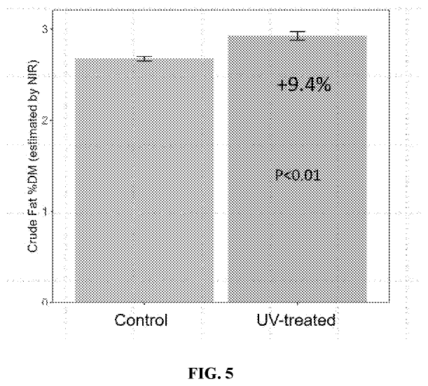

Disclosed herein are novel methods leading to surprisingly large increases in commercial crop energy content, including lipid content. In some embodiments, the methods herein treat grass seeds, such as ryegrass seeds, with UV light. Ryegrass grown from treated seeds shows a 13 to 17% increase in crude fat content, an 18% to 37% increase in shoot fresh weight, a 21% increase in tiller number, an 84% increase in non-structural carbohydrates, and a 2% increase in metabolizable energy compared to ryegrass grown from untreated seeds (FIGS. 1-3 and Tables 1-2). Similarly, corn sileage grown from treated seeds shows a 5% increase in metabolizable energy and a 9.4% increase in crude fat concentration compared to corn grown from untreated seeds (FIGS. 4-5).

Known methods of increasing the energy or lipid content of plants typically involve the use of genetic engineering to create transgenic plants. The disclosed methods present an effective alternative or complement to the use of transgenic plants. Additionally, the demonstrated magnitude of these increases are unusually large. Accordingly, the claimed methods present a novel and efficient solution for improving the energetic plant performance of crops, decreasing the land area needed to effectively graze animals, and decreasing harmful greenhouse gas emissions.

### I. METHODS

In certain aspects, described herein is a method of increasing total lipid percentage in a plant, the method comprising: 1) administering UV-B light to a plant seed, 2) sowing the plant seed, 3) growing the plant from the plant seed, and 4) harvesting the plant. In some embodiments, harvesting a plant comprises an animal grazing the plant.

Also described herein is a method of increasing the total lipid percentage in a plant, the method comprising: 1) administering UV-B light to a plant seed, 2) sowing the plant seed, and 3) growing the plant from the plant seed.

Also described herein is a method of increasing the total lipid percentage in a diet of an animal, the method comprising: 1) administering UV-B light to a plant seed, 2) sowing the plant seed, and 3) growing a plant from the plant seed, wherein the plant is the diet.

Also described herein is a method of increasing the total lipid percentage in a field for grazing, the method comprising: 1) administering UV-B light to a plurality of plant seeds, 2) sowing the plurality of plant seeds in the field, and 3) growing plants from the plurality of plant seeds.

Also described herein is a method of increasing the total lipid percentage of plants in a field, the method comprising: 1) administering UV-B light to a plurality of plant seeds, 2) sowing the plurality of plant seeds in the field, and 3) growing the plants from the plurality of plant seeds.

Also described herein is a method of increasing the total lipid percentage of plants in a field for grazing, the method comprising: 1) administering UV-B light to a plurality of plant seeds, 2) sowing the plurality of plant seeds in the field, 3) growing the plants from the plurality of plant seeds, and 4) grazing animals in the field.

Also described herein is a method of increasing the total lipid percentage of grazing animals, the method comprising 1) administering UV-B light to a plurality of plant seeds, 2) sowing the plurality of plant seeds in a field, 3) growing plant from the plurality of plant seeds, and 4) grazing the animals on the field.

Also described herein is a method of increasing the total energy content per unit of feed consumed by an animal, the method comprising 1) administering UV-B light to a plurality of plant seeds, 2) sowing the plurality of plant seeds in a field, 3) growing plant from the plurality of plant seeds, and 4) grazing the animals on the field.

Also described herein is a method of increasing metabolizable energy in a plant, the method comprising: 1) administering UV-B light to a plant seed, 2) sowing the plant seed, 3) growing the plant from the plant seed, and 4) harvesting the plant. In some embodiments, harvesting a plant comprises an animal grazing the plant.

Also described herein is a method of increasing metabolizable energy in a plant, the method comprising: 1) administering UV-B light to a plant seed, 2) sowing the plant seed, and 3) growing the plant from the plant seed.

Also described herein is a method of increasing non-structural carbohydrates in a plant, the method comprising: 1) administering UV-B light to a plant seed, 2) sowing the plant seed, 3) growing the plant from the plant seed, and 4) harvesting the plant. In some embodiments, harvesting a plant comprises an animal grazing the plant.

Also described herein is a method of increasing non-structural carbohydrates in a plant, the method comprising: 1) administering UV-B light to a plant seed, 2) sowing the plant seed, 3) growing the plant from the plant seed.

Also described herein is a method of decreasing an area of field required for feeding grazing animals, the method comprising: 1) administering UV-B light to a plant seed, 2) sowing the plant seed, 3) growing plant from the plant seed, and 4) grazing the animals on the field.

Also described herein is a method of decreasing an area of field required to supply the dietary needs of grazing animals, the method comprising: 1) administering UV-B light to a plant seed, 2) sowing the plant seed, 3) growing plant from the plant seed, and 4) grazing the animals on the field, wherein the total area required to supply the dietary needs of the grazing animals is decreased compared to a field of plants grown from plant seeds not administered the UV-B light.

Also described herein is a method of reducing the methane production of grazing animals, the method comprising 1) administering UV-B light to a plurality of plant seeds, 2) sowing the plurality of plant seeds in a field, 3) growing plants from the plant seeds, and 4) grazing the animals on the field or feed the plants to the animals.

Also described herein is a method of reducing the methane production of animals, the method comprising 1) administering UV-B light to a plurality of plant seeds, 2) sowing the plurality of plant seeds in a field, 3) growing plants from the plant seeds, 4) harvesting the plants, and 5) feeding the animals the plants.

Also described herein is a method of sequestering carbon from the atmosphere, the method comprising: 1) administering UV-B light to a plant seed, 2) sowing the plant seed, and 3) growing the plant from the plant seed.

In certain aspects, described herein is a method of increasing the total lipid percent in a grass by administering UV-B light to the seed prior to sowing. In some embodiments, the grass comprises a member of the Poaceae family. In some embodiments, the grass comprises a forage grass. In some embodiments, the grass comprises a ryegrass or a bluegrass. In some embodiments, the grass comprises ryegrass.

In some embodiments, the lipid comprises a triacylglycerol. In some embodiments, the lipid comprises a fatty acid. In some embodiments, the fatty acid comprises palmitic acid, palmitoleic acid, stearic acid, oleic acid, linoleic acid, linolenic acid, arachidic acid, gondoic acid, behenic acid, or erucic acid. In some embodiments, the total lipid percent is measured by gas chromatography. In some embodiments, the total lipid percent is measured by near infrared spectroscopy.

Also described herein is a method of increasing the rate of photosynthesis in a plant, the method comprising: administering a light treatment comprising UV-B to a seed and increasing the rate of photosynthesis, wherein the increased net rate of photosynthesis is measured by infrared gas analysis. In some embodiments, the rate of photosynthesis is increased in a plant compared to a plant wherein the seed was not administered UV-B light prior to sowing. In some embodiments, the rate of photosynthesis is increased by at least 1%, 2%, 3%, 4%, 5%, 6%, 7%, 8%, 9%, 10% or more.

Also described herein is a method of improving a field for grazing, the method comprising sowing a plurality of ryegrass seeds in the field, wherein the plurality of ryegrass seeds were administered UV-B light prior to sowing. In some embodiments, the field is improved compared to a field wherein the plurality of ryegrass seeds was not administered UV-B light prior to sowing. In some embodiments, the field is improved by at least 1%, 2%, 3%, 4%, 5%, 6%, 7%, 8%, 9%, 10% or more. In some embodiments, improved comprises an increase in the lipid content of the ryegrass grown in the field. In some embodiments, improved comprises decreasing the area of land required for feeding grazing animals.

Also described herein is a method of increasing the lipid content in a field for grazing, the method comprising sowing a plurality of ryegrass seeds in the field, wherein the plurality of ryegrass seeds were administered UV-B light prior to sowing. In some embodiments, the lipid content in the field is improved compared to a field wherein the plurality of ryegrass seeds was not administered UV-B light prior to sowing. In some embodiments, the lipid content in the field is improved by at least 1%, 2%, 3%, 4%, 5%, 6%, 7%, 8%, 9%, 10% or more.

Also described herein is a method of decreasing the area of land required for feeding grazing animals, the method comprising sowing a plurality of ryegrass seeds in the field, wherein the plurality of ryegrass seeds were administered UV-B light prior to sowing. Also described herein is a method of increasing the lipid content of grazing animals 1) administering a light treatment comprising UV-B to a plurality of ryegrass seeds, 2) sowing the plurality of ryegrass seeds in a field, 3) grazing the animals on the field. Also described herein is a method of increasing the lipid content of grazing animals 1) administering a light treatment comprising UV-B to a plurality of soybean seeds, 2) sowing the plurality of soybean seeds in a field, 3) feeding the resultant plants to the animals. Also described herein is a method of sequestering carbon from the atmosphere using a non-transgenic plant, the method comprising: 1) administering a light treatment comprising UV-B to a plurality of seeds, and 2) sowing the plurality of seeds in a field. Also described herein is a method of reducing the methane production of grazing animals, the method comprising 1) administering a light treatment comprising UV-B to a plurality of grass seeds, 2) sowing the plurality of grass seeds in a field, 3) grazing the animals on the field.

Also described herein is a method of reducing the methane production of animals, the method comprising 1) administering UV-B light to a plurality of plant seeds, 2) sowing the plurality of plant seeds in a field, 3) growing plants from the plant seeds, 4) harvesting the plants, and 5) feeding the animals the plants.

In some embodiments, methane production is reduced by at least about 1%, 2%, 3%, 4%, 5%, 6%, 7%, 8%, 9%, 10%, 11%, 12%, 13%, 14%, 15%, 16%, 17%, 18%, 19% or 20% as compared to a grazing animal grazed on a field not sown with grass seeds administered UV-B. In some embodiments, the grazing animal is a cow or a sheep.

### A. UV Light

Throughout this specification the term “ultraviolet (UV) irradiation” should be taken as meaning electromagnetic radiation with a wavelength shorter than visible light, but longer than X-rays, and is in between the range of 10 nm to 400 nm (corresponding to 3 eV to 124 eV). The ultraviolet (UV) irradiation spectrum is considered to be invisible to humans, and therefore differentiated from visible light in the spectrum of about 400 nm to 700 nm.

The ultraviolet spectrum can be further broken down into UV-A (400-320 nm), UV-B (320-280 nm) and UV-C (280-100 nm).

In some embodiments, use of a wavelength or wavelengths in a specific and narrow focused range within UV radiation between about 275-310 nm leads to beneficial results. In some embodiments, use of a wavelength or wavelengths in a specific and narrow focused range within UV-B radiation between 280-310 nm leads to beneficial results. In some embodiments, part of the UV-B spectrum above about 310 nm does not lead to the beneficial results seen. As will be discussed further, the UV-B spectrum covers 280 nm to about 315 nm (however, defined separations between UV wavebands are approximate, and are subject to at least two common variations in the literature, i.e. including an upper limit for UV-B of 320 nm (IARC monographs on the evaluation of carcinogenic risks to humans. Volume 55-Solar and ultraviolet radiation; Chapter 1; Exposure data (1992)). It is possible that broader treatment within the UV-B spectrum or uncontrolled UV treatment may lead to deleterious results.

Without wishing to be bound by theory, the commercial end result of an increased total lipid percentage, increased metabolizable energy, increased non-structural carbohydrates, decreased area of field required for feeding grazing animals, decreased area of field required to supply the dietary needs of grazing animals, reduced methane production of grazing animals, increased carbon sequestration from the atmosphere, or increased crude fat concentration is thought be at least partially attributed to an exposure of a plant seed to UV light.

In some embodiments, using UV radiation outside of the UV-B range (for example the UV-A or UV-C wavelengths) does not lead to beneficial results. In some embodiments, beneficial effects dramatically diminish or disappear entirely when moving out of the UV-B spectrum, for instance into the UV-A spectrum (400 to 315 nm).

Methods as described herein, in some embodiments, comprise exposure to UV wavelength of about 280 to about 305 nm. In some embodiments, the beneficial effects are most pronounced within a narrower band of the UV-B spectrum, particularly between 280-305 nm.

In some embodiments, beneficial results are still seen beyond 305 nm, but the beneficial results drop sharply after moving beyond a wavelength of about 310 nm. For example, a UV light treatment peaking at 319 nm is still within the UV-B waveband of the spectrum, yet do not appear to produce desired effects. The present disclosure, in some embodiments, uses wavelengths in the short-wave range of the UV-B spectrum, a proportion of which exist outside of the natural spectrum of sunlight that reach the earth's surface. In some embodiments, UV treatment in the UV-A spectrum (at 354 nm) or treatment in the UV-C spectrum (at 270 nm) is not effective to improve plant performance.

In some embodiments, the method includes exposure to a peak UV wavelength of about 280 to about 290 nm. In some embodiments, treatment with UV light peaking between 280-290 nm showed promising results. In some embodiments, the method includes only a specific wavelength (or at least a wavelength peak) between 280-310 nm. In some embodiments, methods as described herein comprise a small amount of UV light that extends partially outside of the 280-310 nm range. In some embodiments, methods comprise insignificant background irradiation. This effect would be minor and would be appreciated by someone skilled in the art to have no real influence on the disclosure's benefits.

Methods as described herein comprise administration of UV in a range of about 275 nm to about 320 nm. In some embodiments, UV is administered at 280 nm (±5 nm), 286 nm (±5 nm), 294 nm (±5 nm), or about 317 nm. The UV can be about 280 nm, about 281 nm, about 282 nm, about 283 nm, about 284 nm, about 285 nm, about 286 nm, about 287 nm, about 288 nm, about 289 nm, about 290 nm, about 291 nm, about 292 nm, about 293 nm, about 294 nm, about 295 nm, about 296 nm, about 297 nm, about 298 nm, about 299 nm, about 300 nm, about 301 nm, about 302 nm, about 303 nm, about 304 nm, about 305 nm, about 306 nm, about 307 nm, about 308 nm, about 309 nm, about 310 nm, about 311 nm, about 312 nm, about 313 nm, about 314 nm, about 315 nm, about 316 nm, about 317 nm, about 318 nm, about 319 nm, or about 320 nm. In some embodiments, UV is peaking at 280 nm (±5 nm), 286 nm (±5 nm), 294 nm (±5 nm), or about 317 nm. The UV can be about 280 nm, about 281 nm, about 282 nm, about 283 nm, about 284 nm, about 285 nm, about 286 nm, about 287 nm, about 288 nm, about 289 nm, about 290 nm, about 291 nm, about 292 nm, about 293 nm, about 294 nm, about 295 nm, about 296 nm, about 297 nm, about 298 nm, about 299 nm, about 300 nm, about 301 nm, about 302 nm, about 303 nm, about 304 nm, about 305 nm, about 306 nm, about 307 nm, about 308 nm, about 309 nm, about 310 nm, about 311 nm, about 312 nm, about 313 nm, about 314 nm, about 315 nm, about 316 nm, about 317 nm, about 318 nm, about 319 nm, or about 320 nm. In some embodiments, the UV is administered or peaking in a range of about 280 nm to about 290 nm, about 280 nm to about 300 nm, about 280 nm to about 310 nm, about 280 nm to about 320 nm, about 290 nm to about 300 nm, about 290 nm to about 310 nm, about 290 nm to about 320 nm, about 300 nm to about 310 nm, about 300 nm to about 320 nm, or about 310 nm to about 320 nm. In some embodiments, the UV is administered or peaking in a range of 280 nm (±5 nm) to 284 nm (±5 nm), 279 nm (±5 nm) to about 288 nm, about 289 nm to about 300 nm, or 286 nm (±5 nm) to about 305 nm. In some embodiments, UV is peaking at 282 nm. In some embodiments, UV is peaking at 292 nm.

In some embodiments, the wavelength within the 280-310 nm range during the method treatment for a given plant species is altered. In some embodiments, a combination of different wavelengths within the UV spectrum are concurrently used.

In some embodiments, methods as described herein do not comprise the use of other UV wavelengths such as UV-A or UV-C in combination with the specific UV-B treatment. In some embodiments, other wavelengths outside of the 280-310 nm UV-B treatment do not comprise part of the methods as described herein. In some embodiments, there is a significant advantage over treatment methods which use multiple wavelengths in more than one spectrum.

The preferred dosage regime(s) of UV light may vary and consider various parameters including, but not limited to, the type of crop, the intensity of the UV light (W m⁻² s⁻¹), the length of treatment (days) and the rest period (on/off) between each UV application during treatment.

In some embodiments, the dose of UV light administered is measured in terms of energy per area over time. In some embodiments, the dose of UV is in a range of about 0.3 kJ m⁻² h⁻¹ to about 3.0 kJ m⁻² h⁻¹, 2.0 kJ m⁻² h⁻¹ to about 12.0 kJ m⁻² h⁻¹, 0.1 kJ m⁻² h⁻¹ to about 1.0 kJ m⁻² h⁻¹, 2 kJ m⁻² d⁻¹ to about 10 kJ m 2 d⁻¹, 1.2 kJ m⁻² d⁻¹ to about 7 kJ m⁻² d⁻¹, 0.1 kJ m⁻² h⁻¹, about 0.2 kJ m⁻² h⁻¹, about 0.3 kJ m⁻² h⁻¹, about 0.4 kJ m⁻² h⁻¹, about 0.5 kJ m⁻² h⁻¹, about 0.6 kJ m⁻² h⁻¹, about 0.7 kJ m⁻² h⁻¹, about 0.8 kJ m⁻² h⁻¹, about 0.9 kJ m⁻² h⁻¹, or about 1.0 kJ m⁻² h⁻¹.

In some embodiments, a duration of administering UV light is about 0.5 minutes to about 180 minutes. In some embodiments, a duration of administering UV light is about 0.5 minutes to about 1 minute, about 0.5 minutes to about 3 minutes, about 0.5 minutes to about 5 minutes, about 0.5 minutes to about 10 minutes, about 0.5 minutes to about 15 minutes, about 0.5 minutes to about 20 minutes, about 0.5 minutes to about 30 minutes, about 0.5 minutes to about 45 minutes, about 0.5 minutes to about 60 minutes, about 0.5 minutes to about 120 minutes, about 0.5 minutes to about 180 minutes, about 1 minute to about 3 minutes, about 1 minute to about 5 minutes, about 1 minute to about 10 minutes, about 1 minute to about 15 minutes, about 1 minute to about 20 minutes, about 1 minute to about 30 minutes, about 1 minute to about 45 minutes, about 1 minute to about 60 minutes, about 1 minute to about 120 minutes, about 1 minute to about 180 minutes, about 3 minutes to about 5 minutes, about 3 minutes to about 10 minutes, about 3 minutes to about 15 minutes, about 3 minutes to about 20 minutes, about 3 minutes to about 30 minutes, about 3 minutes to about 45 minutes, about 3 minutes to about 60 minutes, about 3 minutes to about 120 minutes, about 3 minutes to about 180 minutes, about 5 minutes to about 10 minutes, about 5 minutes to about 15 minutes, about 5 minutes to about 20 minutes, about 5 minutes to about 30 minutes, about 5 minutes to about 45 minutes, about 5 minutes to about 60 minutes, about 5 minutes to about 120 minutes, about 5 minutes to about 180 minutes, about 10 minutes to about 15 minutes, about 10 minutes to about 20 minutes, about 10 minutes to about 30 minutes, about 10 minutes to about 45 minutes, about 10 minutes to about 60 minutes, about 10 minutes to about 120 minutes, about 10 minutes to about 180 minutes, about 15 minutes to about 20 minutes, about 15 minutes to about 30 minutes, about 15 minutes to about 45 minutes, about 15 minutes to about 60 minutes, about 15 minutes to about 120 minutes, about 15 minutes to about 180 minutes, about 20 minutes to about 30 minutes, about 20 minutes to about 45 minutes, about 20 minutes to about 60 minutes, about 20 minutes to about 120 minutes, about 20 minutes to about 180 minutes, about 30 minutes to about 45 minutes, about 30 minutes to about 60 minutes, about 30 minutes to about 120 minutes, about 30 minutes to about 180 minutes, about 45 minutes to about 60 minutes, about 45 minutes to about 120 minutes, about 45 minutes to about 180 minutes, about 60 minutes to about 120 minutes, about 60 minutes to about 180 minutes, or about 120 minutes to about 180 minutes. In some embodiments, a duration of administering UV light is about 0.5 minutes, about 1 minute, about 3 minutes, about 5 minutes, about 10 minutes, about 15 minutes, about 20 minutes, about 30 minutes, about 45 minutes, about 60 minutes, about 120 minutes, or about 180 minutes. In some embodiments, a duration of administering UV light is at least about 0.5 minutes, about 1 minute, about 3 minutes, about 5 minutes, about 10 minutes, about 15 minutes, about 20 minutes, about 30 minutes, about 45 minutes, about 60 minutes, or about 120 minutes. In some embodiments, a duration of administering UV light is at most about 1 minute, about 3 minutes, about 5 minutes, about 10 minutes, about 15 minutes, about 20 minutes, about 30 minutes, about 45 minutes, about 60 minutes, about 120 minutes, or about 180 minutes. In some embodiments, a duration of administering UV comprises at least or about 1 day or at least 14 days. In some embodiments, a duration of administering UV is about 1 day, about 2 days, about 3 days, about 4 days, about 5 days, about 6 days, or about 7 days.

In some embodiments, the dose of UV light administered is measured in terms of mols per area or Photosynthetically Active Radiation. In some embodiments, the dose is about 10 μmol m⁻² to about 500 μmol m⁻². In some embodiments, the dose is about 10 μmol m⁻² to about 50 μmol m⁻², about 10 μmol m⁻² to about 100 μmol m⁻², about 10 μmol m⁻² to about 150 umol m⁻², about 10 μmol m⁻² to about 200 μmol m⁻², about 10 μmol m⁻² to about 250 μmol m⁻², about 10 μmol m⁻² to about 300 μmol m⁻², about 10 μmol m⁻² to about 350 μmol m⁻², about 10 umol m⁻² to about 400 μmol m⁻², about 10 μmol m⁻² to about 500 μmol m⁻², about 50 μmol m⁻² to about 100 μmol m⁻², about 50 μmol m⁻² to about 150 μmol m⁻², about 50 μmol m⁻² to about 200 umol m⁻², about 50 μmol m⁻² to about 250 μmol m⁻², about 50 μmol m⁻² to about 300 μmol m⁻², about 50 μmol m⁻² to about 350 μmol m⁻², about 50 μmol m⁻² to about 400 μmol m⁻², about 50 umol m⁻² to about 500 μmol m⁻², about 100 μmol m⁻² to about 150 μmol m⁻², about 100 μmol m⁻² to about 200 μmol m⁻², about 100 μmol m⁻² to about 250 μmol m⁻², about 100 μmol m⁻² to about 300 μmol m⁻², about 100 μmol m⁻² to about 350 μmol m⁻², about 100 μmol m⁻² to about 400 μmol m⁻², about 100 μmol m⁻² to about 500 μmol m⁻², about 150 μmol m⁻² to about 200 μmol m⁻², about 150 μmol m⁻² to about 250 μmol m⁻², about 150 μmol m⁻² to about 300 μmol m⁻², about 150 μmol m⁻² to about 350 μmol m⁻², about 150 μmol m⁻² to about 400 μmol m⁻², about 150 μmol m⁻² to about 500 μmol m⁻², about 200 μmol m⁻² to about 250 μmol m⁻², about 200 μmol m⁻² to about 300 μmol m⁻², about 200 μmol m⁻² to about 350 μmol m⁻², about 200 μmol m⁻² to about 400 μmol m⁻², about 200 μmol m⁻² to about 500 μmol m⁻², about 250 μmol m⁻² to about 300 μmol m⁻², about 250 μmol m⁻² to about 350 μmol m⁻², about 250 μmol m⁻² to about 400 μmol m⁻², about 250 μmol m⁻² to about 500 μmol m⁻², about 300 μmol m⁻² to about 350 μmol m⁻², about 300 μmol m⁻² to about 400 μmol m⁻², about 300 μmol m⁻² to about 500 μmol m⁻², about 350 μmol m⁻² to about 400 μmol m⁻², about 350 μmol m⁻² to about 500 μmol m⁻², or about 400 μmol m⁻² to about 500 μmol m⁻². In some embodiments, the dose is about 10 μmol m⁻², about 50 μmol m⁻², about 100 μmol m⁻², about 150 μmol m⁻², about 200 μmol m⁻², about 250 μmol m⁻², about 300 umol m⁻², about 350 μmol m⁻², about 400 μmol m⁻², or about 500 μmol m⁻². In some embodiments, the dose is at least about 10 μmol m⁻², about 50 μmol m⁻², about 100 μmol m⁻², about 150 μmol m⁻², about 200 μmol m⁻², about 250 μmol m⁻², about 300 μmol m⁻², about 350 umol m⁻², or about 400 μmol m⁻². In some embodiments, the dose is at most about 50 μmol m⁻², about 100 μmol m⁻², about 150 μmol m⁻², about 200 μmol m⁻², about 250 μmol m⁻², about 300 umol m⁻², about 350 μmol m⁻², about 400 μmol m⁻², or about 500 μmol m⁻².

### B. Plants

Application to a number of plants is consistent with the disclosure herein. In some embodiments, plants subjected to methods as disclosed herein include plant seeds.

In some embodiments, plants subjected to methods as disclosed herein include seedlings, runners, post-seedling plants, leaves, roots, shoot meristems, tillers, or whole plants. In some embodiments whole plants are grown hydroponically or aeroponically. In some instances, the plant is a growing plant that has developed past a seedling stage, such as through the development or reliance upon post-embryonic tissues from the majority of its photosynthesis.

In some embodiments, a plant comprises a member of the Poaceae or Fabaceae family. In some embodiments, the member of the Poaceae family comprises rice, grass, corn, sorghum, wheat, oats, barley, or millet. In some embodiments, the member of the Poaceae family comprises rice, corn, or a grass.

In some embodiments, the grass comprises perennial ryegrass (Lolium perenne), timothy grass (Phleum pratense), tall fescue (Festuca arundinacea), kentucky bluegrass (Poa pratensis), bermuda grass (Cynodon dactylon), alfalfa grass (Medicago sativa), orchard grass (Dactylis glomerata), rye grass (lolium), signal grass (Urochloa panicoides), brome grass (bromus), sudan grass (Sorghum×drummondii), bahia grass (Paspalum notatum), clover (trifolium), meadow fescue (Festuca pratensis), switchgrass (Panicum virgatum), prairie grass (Bromus kalmii), festulolium (hybrid of ryegrass and fescue), Italian ryegrass (Lolium multiflorum), reed canary grass (Phalaris arundinacea), or millet (Panicum miliaceum or Setaria italica). In some embodiments, the grass comprises a ryegrass (including annual ryegrass) or a bluegrass. In some embodiments, the grass comprises a forage grass.

In some embodiments, the member of the Fabaceae family comprises alfalfa, clover, soybean, peas, lentils, vetch, peanut, cowpea, chickpea or, lupins. In some embodiments, the member of the Fabaceae family comprises soybeans.

In some embodiments, the plant comprises a grain. In some embodiments, the plant comprises rice, corn, corn grain, or corn sileage. In some instances, the plant seed or plant is a commercially important plant. The method may also be applicable to a wide variety of other plant types without limitation.

In some embodiments, a plant is selected from the group consisting of fruit and vegetables. In some instances, the plant seedling or plant is selected from the group consisting of green lettuce, soybean, red lettuce, tomato, cucumber, broccoli, herb crops, cannabis, strawberry and eggplant. In some instances, the plant is from at least one of tomato, strawberry, and cannabis.

UV light may be administered at a propagation stage of the plant. In some instances, the propagation stage comprises runners. In some instances, the propagation stage comprises shoots. In some instances, the propagation stage comprises cuttings.

Various cultivation systems for use with methods and devices as described herein may be used. For example, the plant is grown in soil. In some instances, the plant is grown using hydroponics or aeroponics. Plants are grown in controlled greenhouse conditions, such as conventional greenhouse conditions or vertical farming conditions. Alternately, plants are grown outdoors.

### C. Plant Performance Following UV Light Administration

In some embodiments, plant performance of one or more metrics is increased or decreased in plants or plant seeds subjected to methods as disclosed herein as compared to plants or plant seeds not subjected to methods as disclosed herein. In some embodiments, the one or more metrics include one or more of the following metrics.

### 1. Total Lipid Percentage in a Plant

In some embodiments, treatment of the plant seeds with UV light leads to an increase in the lipids produced by the plant. In some embodiments, total lipid percentage is increased by about 1% to about 35%. In some embodiments, total lipid percentage is increased by about 1% to about 3%, about 1% to about 5%, about 1% to about 7%, about 1% to about 10%, about 1% to about 13%, about 1% to about 15%, about 1% to about 17%, about 1% to about 20%, about 1% to about 25%, about 1% to about 30%, about 1% to about 35%, about 3% to about 5%, about 3% to about 7%, about 3% to about 10%, about 3% to about 13%, about 3% to about 15%, about 3% to about 17%, about 3% to about 20%, about 3% to about 25%, about 3% to about 30%, about 3% to about 35%, about 5% to about 7%, about 5% to about 10%, about 5% to about 13%, about 5% to about 15%, about 5% to about 17%, about 5% to about 20%, about 5% to about 25%, about 5% to about 30%, about 5% to about 35%, about 7% to about 10%, about 7% to about 13%, about 7% to about 15%, about 7% to about 17%, about 7% to about 20%, about 7% to about 25%, about 7% to about 30%, about 7% to about 35%, about 10% to about 13%, about 10% to about 15%, about 10% to about 17%, about 10% to about 20%, about 10% to about 25%, about 10% to about 30%, about 10% to about 35%, about 13% to about 15%, about 13% to about 17%, about 13% to about 20%, about 13% to about 25%, about 13% to about 30%, about 13% to about 35%, about 15% to about 17%, about 15% to about 20%, about 15% to about 25%, about 15% to about 30%, about 15% to about 35%, about 17% to about 20%, about 17% to about 25%, about 17% to about 30%, about 17% to about 35%, about 20% to about 25%, about 20% to about 30%, about 20% to about 35%, about 25% to about 30%, about 25% to about 35%, or about 30% to about 35%. In some embodiments, total lipid percentage is increased by about 1%, about 3%, about 5%, about 7%, about 10%, about 13%, about 15%, about 17%, about 20%, about 25%, about 30%, or about 35%. In some embodiments, total lipid percentage is increased by at least about 1%, about 3%, about 5%, about 7%, about 10%, about 13%, about 15%, about 17%, about 20%, about 25%, or about 30%. In some embodiments, total lipid percentage is increased by at most about 3%, about 5%, about 7%, about 10%, about 13%, about 15%, about 17%, about 20%, about 25%, about 30%, or about 35%.

In some embodiments, total lipid percentage in a plant is increased by about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, to 100%, or greater than 100%. In some embodiments, total lipid percentage in a plant is increased by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, to 100%, or at least greater than 100%. In some embodiments, total lipid percentage in a plant is increased by about 1-15, 10-20, 10-30, 10-40, 10-50, 10-60, 10-70, 10-80, 10-90, 10-100, 20-30, 20-40, 20-50, 20-60, 20-70, 20-80, 20-90, 20-100, 30-40, 30-50, 30-60, 30-70, 30-80, 30-90, 30-100, 40-50, 40-60, 40-70, 40-80, 40-90, 40-100, 50-60, 50-70, 50-80, 50-90, 50-100, 60-70, 60-80, 60-90, 60-100, 70-80, 80-90, 90-100, 80-90, 80-100, or 90-100%. In some embodiments, total lipid percentage in a plant is increased by at least 1-15, 10-20, 10-30, 10-40, 10-50, 10-60, 10-70, 10-80, 10-90, 10-100, 20-30, 20-40, 20-50, 20-60, 20-70, 20-80, 20-90, 20-100, 30-40, 30-50, 30-60, 30-70, 30-80, 30-90, 30-100, 40-50, 40-60, 40-70, 40-80, 40-90, 40-100, 50-60, 50-70, 50-80, 50-90, 50-100, 60-70, 60-80, 60-90, 60-100, 70-80, 80-90, 90-100, 80-90, 80-100, or 90-100%.

### 2. Total Lipid Percentage in a Field for Grazing

In some embodiments, treatment of the plant seeds with UV light leads to an increase in the lipids produced by the plant. When the treated seeds are sown in a field for grazing, the total lipid percentage of the field increases. In some embodiments, total lipid percentage in a field for grazing is increased by about 1% to about 35%. In some embodiments, total lipid percentage in a field for grazing is increased by about 1% to about 3%, about 1% to about 5%, about 1% to about 7%, about 1% to about 10%, about 1% to about 13%, about 1% to about 15%, about 1% to about 17%, about 1% to about 20%, about 1% to about 25%, about 1% to about 30%, about 1% to about 35%, about 3% to about 5%, about 3% to about 7%, about 3% to about 10%, about 3% to about 13%, about 3% to about 15%, about 3% to about 17%, about 3% to about 20%, about 3% to about 25%, about 3% to about 30%, about 3% to about 35%, about 5% to about 7%, about 5% to about 10%, about 5% to about 13%, about 5% to about 15%, about 5% to about 17%, about 5% to about 20%, about 5% to about 25%, about 5% to about 30%, about 5% to about 35%, about 7% to about 10%, about 7% to about 13%, about 7% to about 15%, about 7% to about 17%, about 7% to about 20%, about 7% to about 25%, about 7% to about 30%, about 7% to about 35%, about 10% to about 13%, about 10% to about 15%, about 10% to about 17%, about 10% to about 20%, about 10% to about 25%, about 10% to about 30%, about 10% to about 35%, about 13% to about 15%, about 13% to about 17%, about 13% to about 20%, about 13% to about 25%, about 13% to about 30%, about 13% to about 35%, about 15% to about 17%, about 15% to about 20%, about 15% to about 25%, about 15% to about 30%, about 15% to about 35%, about 17% to about 20%, about 17% to about 25%, about 17% to about 30%, about 17% to about 35%, about 20% to about 25%, about 20% to about 30%, about 20% to about 35%, about 25% to about 30%, about 25% to about 35%, or about 30% to about 35%. In some embodiments, total lipid percentage in a field for grazing is increased by about 1%, about 3%, about 5%, about 7%, about 10%, about 13%, about 15%, about 17%, about 20%, about 25%, about 30%, or about 35%. In some embodiments, total lipid percentage in a field for grazing is increased by at least about 1%, about 3%, about 5%, about 7%, about 10%, about 13%, about 15%, about 17%, about 20%, about 25%, or about 30%. In some embodiments, total lipid percentage in a field for grazing is increased by at most about 3%, about 5%, about 7%, about 10%, about 13%, about 15%, about 17%, about 20%, about 25%, about 30%, or about 35%.

In some embodiments, total lipid percentage in a field for grazing is increased by about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, to 100%, or greater than 100%. In some embodiments, total lipid percentage in a field for grazing is increased by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, to 100%, or at least greater than 100%. In some embodiments, total lipid percentage in a field for grazing is increased by about 1-15, 10-20, 10-30, 10-40, 10-50, 10-60, 10-70, 10-80, 10-90, 10-100, 20-30, 20-40, 20-50, 20-60, 20-70, 20-80, 20-90, 20-100, 30-40, 30-50, 30-60, 30-70, 30-80, 30-90, 30-100, 40-50, 40-60, 40-70, 40-80, 40-90, 40-100, 50-60, 50-70, 50-80, 50-90, 50-100, 60-70, 60-80, 60-90, 60-100, 70-80, 80-90, 90-100, 80-90, 80-100, or 90-100%. In some embodiments, total lipid percentage in a field for grazing is increased by at least 1-15, 10-20, 10-30, 10-40, 10-50, 10-60, 10-70, 10-80, 10-90, 10-100, 20-30, 20-40, 20-50, 20-60, 20-70, 20-80, 20-90, 20-100, 30-40, 30-50, 30-60, 30-70, 30-80, 30-90, 30-100, 40-50, 40-60, 40-70, 40-80, 40-90, 40-100, 50-60, 50-70, 50-80, 50-90, 50-100, 60-70, 60-80, 60-90, 60-100, 70-80, 80-90, 90-100, 80-90, 80-100, or 90-100%.

### 3. Total Lipid Percentage of Grazing Animals

In some embodiments, treatment of the plant seeds with UV light leads to an increase in the lipids produced by the plant. When the treated seeds are sown in a field for grazing, the total lipid percentage of the field increases. When grazing animals graze on the field or eat plants harvested from the field, the total lipid percentage of the grazing animals increases.

In some embodiments, total lipid percentage of grazing animals is increased by about 1% to about 10%. In some embodiments, total lipid percentage of grazing animals is increased by about 1% to about 10%, about 1% to about 2%, about 1% to about 9%, about 1% to about 3%, about 1% to about 8%, about 1% to about 4%, about 1% to about 7%, about 1% to about 5%, about 1% to about 6%, about 10% to about 2%, about 10% to about 9%, about 10% to about 3%, about 10% to about 8%, about 10% to about 4%, about 10% to about 7%, about 10% to about 5%, about 10% to about 6%, about 2% to about 9%, about 2% to about 3%, about 2% to about 8%, about 2% to about 4%, about 2% to about 7%, about 2% to about 5%, about 2% to about 6%, about 9% to about 3%, about 9% to about 8%, about 9% to about 4%, about 9% to about 7%, about 9% to about 5%, about 9% to about 6%, about 3% to about 8%, about 3% to about 4%, about 3% to about 7%, about 3% to about 5%, about 3% to about 6%, about 8% to about 4%, about 8% to about 7%, about 8% to about 5%, about 8% to about 6%, about 4% to about 7%, about 4% to about 5%, about 4% to about 6%, about 7% to about 5%, about 7% to about 6%, or about 5% to about 6%. In some embodiments, total lipid percentage of grazing animals is increased by about 1%, about 10%, about 2%, about 9%, about 3%, about 8%, about 4%, about 7%, about 5%, or about 6%. In some embodiments, total lipid percentage of grazing animals is increased by at least about 1%, about 10%, about 2%, about 9%, about 3%, about 8%, about 4%, about 7%, or about 5%. In some embodiments, total lipid percentage of grazing animals is increased by at most about 10%, about 2%, about 9%, about 3%, about 8%, about 4%, about 7%, about 5%, or about 6%.

In some embodiments, total lipid percentage of grazing animals is increased by about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, to 100%, or greater than 100%. In some embodiments, total lipid percentage of grazing animals is increased by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, to 100%, or at least greater than 100%. In some embodiments, total lipid percentage of grazing animals is increased by about 1-15, 10-20, 10-30, 10-40, 10-50, 10-60, 10-70, 10-80, 10-90, 10-100, 20-30, 20-40, 20-50, 20-60, 20-70, 20-80, 20-90, 20-100, 30-40, 30-50, 30-60, 30-70, 30-80, 30-90, 30-100, 40-50, 40-60, 40-70, 40-80, 40-90, 40-100, 50-60, 50-70, 50-80, 50-90, 50-100, 60-70, 60-80, 60-90, 60-100, 70-80, 80-90, 90-100, 80-90, 80-100, or 90-100%. In some embodiments, total lipid percentage of grazing animals is increased by at least 1-15, 10-20, 10-30, 10-40, 10-50, 10-60, 10-70, 10-80, 10-90, 10-100, 20-30, 20-40, 20-50, 20-60, 20-70, 20-80, 20-90, 20-100, 30-40, 30-50, 30-60, 30-70, 30-80, 30-90, 30-100, 40-50, 40-60, 40-70, 40-80, 40-90, 40-100, 50-60, 50-70, 50-80, 50-90, 50-100, 60-70, 60-80, 60-90, 60-100, 70-80, 80-90, 90-100, 80-90, 80-100, or 90-100%.

### 4. Total Energy Content Per Unit of Feed Consumed

In some embodiments, treatment of the plant seeds with UV light leads to an increase in the total energy content per unit of feed consumed by an animal. In some embodiments, the total energy content per unit of feed consumed by an animal is increased by about 1% to about 10%, about 1% to about 2%, about 1% to about 9%, about 1% to about 3%, about 1% to about 8%, about 1% to about 4%, about 1% to about 7%, about 1% to about 5%, about 1% to about 6%, about 10% to about 2%, about 10% to about 9%, about 10% to about 3%, about 10% to about 8%, about 10% to about 4%, about 10% to about 7%, about 10% to about 5%, about 10% to about 6%, about 2% to about 9%, about 2% to about 3%, about 2% to about 8%, about 2% to about 4%, about 2% to about 7%, about 2% to about 5%, about 2% to about 6%, about 9% to about 3%, about 9% to about 8%, about 9% to about 4%, about 9% to about 7%, about 9% to about 5%, about 9% to about 6%, about 3% to about 8%, about 3% to about 4%, about 3% to about 7%, about 3% to about 5%, about 3% to about 6%, about 8% to about 4%, about 8% to about 7%, about 8% to about 5%, about 8% to about 6%, about 4% to about 7%, about 4% to about 5%, about 4% to about 6%, about 7% to about 5%, about 7% to about 6%, or about 5% to about 6%. In some embodiments, total lipid percentage of grazing animals is increased by about 1%, about 10%, about 2%, about 9%, about 3%, about 8%, about 4%, about 7%, about 5%, or about 6%. In some embodiments, total lipid percentage of grazing animals is increased by at least about 1%, about 10%, about 2%, about 9%, about 3%, about 8%, about 4%, about 7%, or about 5%. In some embodiments, total lipid percentage of grazing animals is increased by at most about 10%, about 2%, about 9%, about 3%, about 8%, about 4%, about 7%, about 5%, or about 6%.

In some embodiments, total energy content per unit of feed consumed by an animal is increased by about 1 MJ/Kg to about 10 MJ/Kg of dry matter. In some embodiments, total energy content per unit of feed consumed by an animal is increased by about 1 MJ/Kg to about 10 MJ/Kg of dry matter, about 1 MJ/Kg to about 2 MJ/Kg of dry matter, about 1 MJ/Kg to about 9 MJ/Kg of dry matter, about 1 MJ/Kg to about 3 MJ/Kg of dry matter, about 1 MJ/Kg to about 8 MJ/Kg of dry matter, about 1 MJ/Kg to about 4 MJ/Kg of dry matter, about 1 MJ/Kg to about 7 MJ/Kg of dry matter, about 1 MJ/Kg to about 5 MJ/Kg of dry matter, about 1 MJ/Kg to about 6 MJ/Kg of dry matter, about 10 MJ/Kg of dry matter to about 2 MJ/Kg of dry matter, about 10 MJ/Kg of dry matter to about 9 MJ/Kg of dry matter, about 10 MJ/Kg of dry matter to about 3 MJ/Kg of dry matter, about 10 MJ/Kg of dry matter to about 8 MJ/Kg of dry matter, about 10 MJ/Kg of dry matter to about 4 MJ/Kg of dry matter, about 10 MJ/Kg of dry matter to about 7 MJ/Kg of dry matter, about 10 MJ/Kg of dry matter to about 5 MJ/Kg of dry matter, about 10 MJ/Kg of dry matter to about 6 MJ/Kg of dry matter, about 2 MJ/Kg of dry matter to about 9 MJ/Kg of dry matter, about 2 MJ/Kg of dry matter to about 3 MJ/Kg of dry matter, about 2 MJ/Kg of dry matter to about 8 MJ/Kg of dry matter, about 2 MJ/Kg of dry matter to about 4 MJ/Kg of dry matter, about 2 MJ/Kg of dry matter to about 7 MJ/Kg of dry matter, about 2 MJ/Kg of dry matter to about 5 MJ/Kg of dry matter, about 2 MJ/Kg of dry matter to about 6 MJ/Kg of dry matter, about 9 MJ/Kg of dry matter to about 3 MJ/Kg of dry matter, about 9 MJ/Kg of dry matter to about 8 MJ/Kg of dry matter, about 9 MJ/Kg of dry matter to about 4 MJ/Kg of dry matter, about 9 MJ/Kg of dry matter to about 7 MJ/Kg of dry matter, about 9 MJ/Kg of dry matter to about 5 MJ/Kg of dry matter, about 9 MJ/Kg of dry matter to about 6 MJ/Kg of dry matter, about 3 MJ/Kg of dry matter to about 8 MJ/Kg of dry matter, about 3 MJ/Kg of dry matter to about 4 MJ/Kg of dry matter, about 3 MJ/Kg of dry matter to about 7 MJ/Kg of dry matter, about 3 MJ/Kg of dry matter to about 5 MJ/Kg of dry matter, about 3 MJ/Kg of dry matter to about 6 MJ/Kg of dry matter, about 8 MJ/Kg of dry matter to about 4 MJ/Kg of dry matter, about 8 MJ/Kg of dry matter to about 7 MJ/Kg of dry matter, about 8 MJ/Kg of dry matter to about 5 MJ/Kg of dry matter, about 8 MJ/Kg of dry matter to about 6 MJ/Kg of dry matter, about 4 MJ/Kg of dry matter to about 7 MJ/Kg of dry matter, about 4 MJ/Kg of dry matter to about 5 MJ/Kg of dry matter, about 4 MJ/Kg of dry matter to about 6 MJ/Kg of dry matter, about 7 MJ/Kg of dry matter to about 5 MJ/Kg of dry matter, about 7 MJ/Kg of dry matter to about 6 MJ/Kg of dry matter, or about 5 MJ/Kg of dry matter to about 6 MJ/Kg of dry matter. In some embodiments, total energy content per unit of feed consumed by an animal is increased by about 1 MJ/Kg, about 10 MJ/Kg of dry matter, about 2 MJ/Kg of dry matter, about 9 MJ/Kg of dry matter, about 3 MJ/Kg of dry matter, about 8 MJ/Kg of dry matter, about 4 MJ/Kg of dry matter, about 7 MJ/Kg of dry matter, about 5 MJ/Kg of dry matter, or about 6 MJ/Kg of dry matter. In some embodiments, total energy content per unit of feed consumed by an animal is increased by at least about 1 MJ/Kg, about 10 MJ/Kg of dry matter, about 2 MJ/Kg of dry matter, about 9 MJ/Kg of dry matter, about 3 MJ/Kg of dry matter, about 8 MJ/Kg of dry matter, about 4 MJ/Kg of dry matter, about 7 MJ/Kg of dry matter, or about 5 MJ/Kg of dry matter. In some embodiments, total energy content per unit of feed consumed by an animal is increased by at most about 10 MJ/Kg of dry matter, about 2 MJ/Kg of dry matter, about 9 MJ/Kg of dry matter, about 3 MJ/Kg of dry matter, about 8 MJ/Kg of dry matter, about 4 MJ/Kg of dry matter, about 7 MJ/Kg of dry matter, about 5 MJ/Kg of dry matter, or about 6 MJ/Kg of dry matter.

In some embodiments, total energy content per unit of feed consumed by an animal in a plant is increased by about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, to 100%, or greater than 100%. In some embodiments, total energy content per unit of feed consumed by an animal in a plant is increased by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, to 100%, or at least greater than 100%. In some embodiments, total energy content per unit of feed consumed by an animal in a plant is increased by about 1-15, 10-20, 10-30, 10-40, 10-50, 10-60, 10-70, 10-80, 10-90, 10-100, 20-30, 20-40, 20-50, 20-60, 20-70, 20-80, 20-90, 20-100, 30-40, 30-50, 30-60, 30-70, 30-80, 30-90, 30-100, 40-50, 40-60, 40-70, 40-80, 40-90, 40-100, 50-60, 50-70, 50-80, 50-90, 50-100, 60-70, 60-80, 60-90, 60-100, 70-80, 80-90, 90-100, 80-90, 80-100, or 90-100%. In some embodiments, total energy content per unit of feed consumed by an animal in a plant is increased by at least 1-15, 10-20, 10-30, 10-40, 10-50, 10-60, 10-70, 10-80, 10-90, 10-100, 20-30, 20-40, 20-50, 20-60, 20-70, 20-80, 20-90, 20-100, 30-40, 30-50, 30-60, 30-70, 30-80, 30-90, 30-100, 40-50, 40-60, 40-70, 40-80, 40-90, 40-100, 50-60, 50-70, 50-80, 50-90, 50-100, 60-70, 60-80, 60-90, 60-100, 70-80, 80-90, 90-100, 80-90, 80-100, or 90-100%.

In some embodiments, total energy content per unit of feed consumed by an animal in a plant is increased by about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, to 100 MJ/Kg of dry matter. In some embodiments, total energy content per unit of feed consumed by an animal in a plant is increased by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, to 100 MJ/Kg of dry matter. In some embodiments, total energy content per unit of feed consumed by an animal in a plant is increased by about 1-15, 10-20, 10-30, 10-40, 10-50, 10-60, 10-70, 10-80, 10-90, 10-100, 20-30, 20-40, 20-50, 20-60, 20-70, 20-80, 20-90, 20-100, 30-40, 30-50, 30-60, 30-70, 30-80, 30-90, 30-100, 40-50, 40-60, 40-70, 40-80, 40-90, 40-100, 50-60, 50-70, 50-80, 50-90, 50-100, 60-70, 60-80, 60-90, 60-100, 70-80, 80-90, 90-100, 80-90, 80-100, or 90-100 MJ/Kg of dry matter. In some embodiments, total energy content per unit of feed consumed by an animal in a plant is increased by at least 1-15, 10-20, 10-30, 10-40, 10-50, 10-60, 10-70, 10-80, 10-90, 10-100, 20-30, 20-40, 20-50, 20-60, 20-70, 20-80, 20-90, 20-100, 30-40, 30-50, 30-60, 30-70, 30-80, 30-90, 30-100, 40-50, 40-60, 40-70, 40-80, 40-90, 40-100, 50-60, 50-70, 50-80, 50-90, 50-100, 60-70, 60-80, 60-90, 60-100, 70-80, 80-90, 90-100, 80-90, 80-100, or 90-100 MJ/Kg of dry matter.

### 5. Metabolizable Energy in a Plant

In some embodiments, treatment of the plant seeds with UV light leads to an increase in the total metabolizable energy in the plant. In some embodiments, the total metabolizable energy is increased by about 1% to about 10%, about 1% to about 2%, about 1% to about 9%, about 1% to about 3%, about 1% to about 8%, about 1% to about 4%, about 1% to about 7%, about 1% to about 5%, about 1% to about 6%, about 10% to about 2%, about 10% to about 9%, about 10% to about 3%, about 10% to about 8%, about 10% to about 4%, about 10% to about 7%, about 10% to about 5%, about 10% to about 6%, about 2% to about 9%, about 2% to about 3%, about 2% to about 8%, about 2% to about 4%, about 2% to about 7%, about 2% to about 5%, about 2% to about 6%, about 9% to about 3%, about 9% to about 8%, about 9% to about 4%, about 9% to about 7%, about 9% to about 5%, about 9% to about 6%, about 3% to about 8%, about 3% to about 4%, about 3% to about 7%, about 3% to about 5%, about 3% to about 6%, about 8% to about 4%, about 8% to about 7%, about 8% to about 5%, about 8% to about 6%, about 4% to about 7%, about 4% to about 5%, about 4% to about 6%, about 7% to about 5%, about 7% to about 6%, or about 5% to about 6%. In some embodiments, total lipid percentage of grazing animals is increased by about 1%, about 10%, about 2%, about 9%, about 3%, about 8%, about 4%, about 7%, about 5%, or about 6%. In some embodiments, total lipid percentage of grazing animals is increased by at least about 1%, about 10%, about 2%, about 9%, about 3%, about 8%, about 4%, about 7%, or about 5%. In some embodiments, total lipid percentage of grazing animals is increased by at most about 10%, about 2%, about 9%, about 3%, about 8%, about 4%, about 7%, about 5%, or about 6%.

In some embodiments, total metabolizable energy is increased by about 1 MJ/Kg to about 10 MJ/Kg of dry matter. In some embodiments, total metabolizable energy is increased by about 1 MJ/Kg to about 10 MJ/Kg of dry matter, about 1 MJ/Kg to about 2 MJ/Kg of dry matter, about 1 MJ/Kg to about 9 MJ/Kg of dry matter, about 1 MJ/Kg to about 3 MJ/Kg of dry matter, about 1 MJ/Kg to about 8 MJ/Kg of dry matter, about 1 MJ/Kg to about 4 MJ/Kg of dry matter, about 1 MJ/Kg to about 7 MJ/Kg of dry matter, about 1 MJ/Kg to about 5 MJ/Kg of dry matter, about 1 MJ/Kg to about 6 MJ/Kg of dry matter, about 10 MJ/Kg of dry matter to about 2 MJ/Kg of dry matter, about 10 MJ/Kg of dry matter to about 9 MJ/Kg of dry matter, about 10 MJ/Kg of dry matter to about 3 MJ/Kg of dry matter, about 10 MJ/Kg of dry matter to about 8 MJ/Kg of dry matter, about 10 MJ/Kg of dry matter to about 4 MJ/Kg of dry matter, about 10 MJ/Kg of dry matter to about 7 MJ/Kg of dry matter, about 10 MJ/Kg of dry matter to about 5 MJ/Kg of dry matter, about 10 MJ/Kg of dry matter to about 6 MJ/Kg of dry matter, about 2 MJ/Kg of dry matter to about 9 MJ/Kg of dry matter, about 2 MJ/Kg of dry matter to about 3 MJ/Kg of dry matter, about 2 MJ/Kg of dry matter to about 8 MJ/Kg of dry matter, about 2 MJ/Kg of dry matter to about 4 MJ/Kg of dry matter, about 2 MJ/Kg of dry matter to about 7 MJ/Kg of dry matter, about 2 MJ/Kg of dry matter to about 5 MJ/Kg of dry matter, about 2 MJ/Kg of dry matter to about 6 MJ/Kg of dry matter, about 9 MJ/Kg of dry matter to about 3 MJ/Kg of dry matter, about 9 MJ/Kg of dry matter to about 8 MJ/Kg of dry matter, about 9 MJ/Kg of dry matter to about 4 MJ/Kg of dry matter, about 9 MJ/Kg of dry matter to about 7 MJ/Kg of dry matter, about 9 MJ/Kg of dry matter to about 5 MJ/Kg of dry matter, about 9 MJ/Kg of dry matter to about 6 MJ/Kg of dry matter, about 3 MJ/Kg of dry matter to about 8 MJ/Kg of dry matter, about 3 MJ/Kg of dry matter to about 4 MJ/Kg of dry matter, about 3 MJ/Kg of dry matter to about 7 MJ/Kg of dry matter, about 3 MJ/Kg of dry matter to about 5 MJ/Kg of dry matter, about 3 MJ/Kg of dry matter to about 6 MJ/Kg of dry matter, about 8 MJ/Kg of dry matter to about 4 MJ/Kg of dry matter, about 8 MJ/Kg of dry matter to about 7 MJ/Kg of dry matter, about 8 MJ/Kg of dry matter to about 5 MJ/Kg of dry matter, about 8 MJ/Kg of dry matter to about 6 MJ/Kg of dry matter, about 4 MJ/Kg of dry matter to about 7 MJ/Kg of dry matter, about 4 MJ/Kg of dry matter to about 5 MJ/Kg of dry matter, about 4 MJ/Kg of dry matter to about 6 MJ/Kg of dry matter, about 7 MJ/Kg of dry matter to about 5 MJ/Kg of dry matter, about 7 MJ/Kg of dry matter to about 6 MJ/Kg of dry matter, or about 5 MJ/Kg of dry matter to about 6 MJ/Kg of dry matter. In some embodiments, total metabolizable energy is increased by about 1 MJ/Kg, about 10 MJ/Kg of dry matter, about 2 MJ/Kg of dry matter, about 9 MJ/Kg of dry matter, about 3 MJ/Kg of dry matter, about 8 MJ/Kg of dry matter, about 4 MJ/Kg of dry matter, about 7 MJ/Kg of dry matter, about 5 MJ/Kg of dry matter, or about 6 MJ/Kg of dry matter. In some embodiments, total metabolizable energy is increased by at least about 1 MJ/Kg, about 10 MJ/Kg of dry matter, about 2 MJ/Kg of dry matter, about 9 MJ/Kg of dry matter, about 3 MJ/Kg of dry matter, about 8 MJ/Kg of dry matter, about 4 MJ/Kg of dry matter, about 7 MJ/Kg of dry matter, or about 5 MJ/Kg of dry matter. In some embodiments, total metabolizable energy is increased by at most about 10 MJ/Kg of dry matter, about 2 MJ/Kg of dry matter, about 9 MJ/Kg of dry matter, about 3 MJ/Kg of dry matter, about 8 MJ/Kg of dry matter, about 4 MJ/Kg of dry matter, about 7 MJ/Kg of dry matter, about 5 MJ/Kg of dry matter, or about 6 MJ/Kg of dry matter.

In some embodiments, total metabolizable energy in a plant is increased by about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, to 100%, or greater than 100%. In some embodiments, total metabolizable energy in a plant is increased by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, to 100%, or at least greater than 100%. In some embodiments, total metabolizable energy in a plant is increased by about 1-15, 10-20, 10-30, 10-40, 10-50, 10-60, 10-70, 10-80, 10-90, 10-100, 20-30, 20-40, 20-50, 20-60, 20-70, 20-80, 20-90, 20-100, 30-40, 30-50, 30-60, 30-70, 30-80, 30-90, 30-100, 40-50, 40-60, 40-70, 40-80, 40-90, 40-100, 50-60, 50-70, 50-80, 50-90, 50-100, 60-70, 60-80, 60-90, 60-100, 70-80, 80-90, 90-100, 80-90, 80-100, or 90-100%. In some embodiments, total metabolizable energy in a plant is increased by at least 1-15, 10-20, 10-30, 10-40, 10-50, 10-60, 10-70, 10-80, 10-90, 10-100, 20-30, 20-40, 20-50, 20-60, 20-70, 20-80, 20-90, 20-100, 30-40, 30-50, 30-60, 30-70, 30-80, 30-90, 30-100, 40-50, 40-60, 40-70, 40-80, 40-90, 40-100, 50-60, 50-70, 50-80, 50-90, 50-100, 60-70, 60-80, 60-90, 60-100, 70-80, 80-90, 90-100, 80-90, 80-100, or 90-100%.

In some embodiments, total metabolizable energy in a plant is increased by about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, to 100 MJ/Kg of dry matter. In some embodiments, total metabolizable energy in a plant is increased by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, to 100 MJ/Kg of dry matter. In some embodiments, total metabolizable energy in a plant is increased by about 1-15, 10-20, 10-30, 10-40, 10-50, 10-60, 10-70, 10-80, 10-90, 10-100, 20-30, 20-40, 20-50, 20-60, 20-70, 20-80, 20-90, 20-100, 30-40, 30-50, 30-60, 30-70, 30-80, 30-90, 30-100, 40-50, 40-60, 40-70, 40-80, 40-90, 40-100, 50-60, 50-70, 50-80, 50-90, 50-100, 60-70, 60-80, 60-90, 60-100, 70-80, 80-90, 90-100, 80-90, 80-100, or 90-100 MJ/Kg of dry matter. In some embodiments, total metabolizable energy in a plant is increased by at least 1-15, 10-20, 10-30, 10-40, 10-50, 10-60, 10-70, 10-80, 10-90, 10-100, 20-30, 20-40, 20-50, 20-60, 20-70, 20-80, 20-90, 20-100, 30-40, 30-50, 30-60, 30-70, 30-80, 30-90, 30-100, 40-50, 40-60, 40-70, 40-80, 40-90, 40-100, 50-60, 50-70, 50-80, 50-90, 50-100, 60-70, 60-80, 60-90, 60-100, 70-80, 80-90, 90-100, 80-90, 80-100, or 90-100 MJ/Kg of dry matter.

### 6. Non-Structural Carbohydrates in a Plant

In some embodiments, treatment of the plant seeds with UV light leads to an increase in total non-structural carbohydrates in the plant. In some embodiments, total non-structural carbohydrates in a plant is increased by about 1% to about 100%. In some embodiments, total non-structural carbohydrates in a plant is increased by about 1% to about 10%, about 1% to about 20%, about 1% to about 30%, about 1% to about 40%, about 1% to about 50%, about 1% to about 60%, about 1% to about 70%, about 1% to about 80%, about 1% to about 84%, about 1% to about 90%, about 1% to about 100%, about 10% to about 20%, about 10% to about 30%, about 10% to about 40%, about 10% to about 50%, about 10% to about 60%, about 10% to about 70%, about 10% to about 80%, about 10% to about 84%, about 10% to about 90%, about 10% to about 100%, about 20% to about 30%, about 20% to about 40%, about 20% to about 50%, about 20% to about 60%, about 20% to about 70%, about 20% to about 80%, about 20% to about 84%, about 20% to about 90%, about 20% to about 100%, about 30% to about 40%, about 30% to about 50%, about 30% to about 60%, about 30% to about 70%, about 30% to about 80%, about 30% to about 84%, about 30% to about 90%, about 30% to about 100%, about 40% to about 50%, about 40% to about 60%, about 40% to about 70%, about 40% to about 80%, about 40% to about 84%, about 40% to about 90%, about 40% to about 100%, about 50% to about 60%, about 50% to about 70%, about 50% to about 80%, about 50% to about 84%, about 50% to about 90%, about 50% to about 100%, about 60% to about 70%, about 60% to about 80%, about 60% to about 84%, about 60% to about 90%, about 60% to about 100%, about 70% to about 80%, about 70% to about 84%, about 70% to about 90%, about 70% to about 100%, about 80% to about 84%, about 80% to about 90%, about 80% to about 100%, about 84% to about 90%, about 84% to about 100%, or about 90% to about 100%. In some embodiments, total non-structural carbohydrates in a plant is increased by about 1%, about 10%, about 20%, about 30%, about 40%, about 50%, about 60%, about 70%, about 80%, about 84%, about 90%, or about 100%. In some embodiments, total non-structural carbohydrates in a plant is increased by at least about 1%, about 10%, about 20%, about 30%, about 40%, about 50%, about 60%, about 70%, about 80%, about 84%, or about 90%. In some embodiments, total non-structural carbohydrates in a plant is increased by at most about 10%, about 20%, about 30%, about 40%, about 50%, about 60%, about 70%, about 80%, about 84%, about 90%, or about 100%.

In some embodiments, total non-structural carbohydrates in a plant is increased by about 1% of plant dry matter percentage to about 100% of plant dry matter percentage. In some embodiments, total non-structural carbohydrates in a plant is increased by about 1% of plant dry matter percentage to about 10% of plant dry matter percentage, about 1% of plant dry matter percentage to about 20% of plant dry matter percentage, about 1% of plant dry matter percentage to about 30% of plant dry matter percentage, about 1% of plant dry matter percentage to about 40% of plant dry matter percentage, about 1% of plant dry matter percentage to about 50% of plant dry matter percentage, about 1% of plant dry matter percentage to about 60% of plant dry matter percentage, about 1% of plant dry matter percentage to about 70% of plant dry matter percentage, about 1% of plant dry matter percentage to about 80% of plant dry matter percentage, about 1% of plant dry matter percentage to about 84% of plant dry matter percentage, about 1% of plant dry matter percentage to about 90% of plant dry matter percentage, about 1% of plant dry matter percentage to about 100% of plant dry matter percentage, about 10% of plant dry matter percentage to about 20% of plant dry matter percentage, about 10% of plant dry matter percentage to about 30% of plant dry matter percentage, about 10% of plant dry matter percentage to about 40% of plant dry matter percentage, about 10% of plant dry matter percentage to about 50% of plant dry matter percentage, about 10% of plant dry matter percentage to about 60% of plant dry matter percentage, about 10% of plant dry matter percentage to about 70% of plant dry matter percentage, about 10% of plant dry matter percentage to about 80% of plant dry matter percentage, about 10% of plant dry matter percentage to about 84% of plant dry matter percentage, about 10% of plant dry matter percentage to about 90% of plant dry matter percentage, about 10% of plant dry matter percentage to about 100% of plant dry matter percentage, about 20% of plant dry matter percentage to about 30% of plant dry matter percentage, about 20% of plant dry matter percentage to about 40% of plant dry matter percentage, about 20% of plant dry matter percentage to about 50% of plant dry matter percentage, about 20% of plant dry matter percentage to about 60% of plant dry matter percentage, about 20% of plant dry matter percentage to about 70% of plant dry matter percentage, about 20% of plant dry matter percentage to about 80% of plant dry matter percentage, about 20% of plant dry matter percentage to about 84% of plant dry matter percentage, about 20% of plant dry matter percentage to about 90% of plant dry matter percentage, about 20% of plant dry matter percentage to about 100% of plant dry matter percentage, about 30% of plant dry matter percentage to about 40% of plant dry matter percentage, about 30% of plant dry matter percentage to about 50% of plant dry matter percentage, about 30% of plant dry matter percentage to about 60% of plant dry matter percentage, about 30% of plant dry matter percentage to about 70% of plant dry matter percentage, about 30% of plant dry matter percentage to about 80% of plant dry matter percentage, about 30% of plant dry matter percentage to about 84% of plant dry matter percentage, about 30% of plant dry matter percentage to about 90% of plant dry matter percentage, about 30% of plant dry matter percentage to about 100% of plant dry matter percentage, about 40% of plant dry matter percentage to about 50% of plant dry matter percentage, about 40% of plant dry matter percentage to about 60% of plant dry matter percentage, about 40% of plant dry matter percentage to about 70% of plant dry matter percentage, about 40% of plant dry matter percentage to about 80% of plant dry matter percentage, about 40% of plant dry matter percentage to about 84% of plant dry matter percentage, about 40% of plant dry matter percentage to about 90% of plant dry matter percentage, about 40% of plant dry matter percentage to about 100% of plant dry matter percentage, about 50% of plant dry matter percentage to about 60% of plant dry matter percentage, about 50% of plant dry matter percentage to about 70% of plant dry matter percentage, about 50% of plant dry matter percentage to about 80% of plant dry matter percentage, about 50% of plant dry matter percentage to about 84% of plant dry matter percentage, about 50% of plant dry matter percentage to about 90% of plant dry matter percentage, about 50% of plant dry matter percentage to about 100% of plant dry matter percentage, about 60% of plant dry matter percentage to about 70% of plant dry matter percentage, about 60% of plant dry matter percentage to about 80% of plant dry matter percentage, about 60% of plant dry matter percentage to about 84% of plant dry matter percentage, about 60% of plant dry matter percentage to about 90% of plant dry matter percentage, about 60% of plant dry matter percentage to about 100% of plant dry matter percentage, about 70% of plant dry matter percentage to about 80% of plant dry matter percentage, about 70% of plant dry matter percentage to about 84% of plant dry matter percentage, about 70% of plant dry matter percentage to about 90% of plant dry matter percentage, about 70% of plant dry matter percentage to about 100% of plant dry matter percentage, about 80% of plant dry matter percentage to about 84% of plant dry matter percentage, about 80% of plant dry matter percentage to about 90% of plant dry matter percentage, about 80% of plant dry matter percentage to about 100% of plant dry matter percentage, about 84% of plant dry matter percentage to about 90% of plant dry matter percentage, about 84% of plant dry matter percentage to about 100% of plant dry matter percentage, or about 90% of plant dry matter percentage to about 100% of plant dry matter percentage. In some embodiments, total non-structural carbohydrates in a plant is increased by about 1% of plant dry matter percentage, about 10% of plant dry matter percentage, about 20% of plant dry matter percentage, about 30% of plant dry matter percentage, about 40% of plant dry matter percentage, about 50% of plant dry matter percentage, about 60% of plant dry matter percentage, about 70% of plant dry matter percentage, about 80% of plant dry matter percentage, about 84% of plant dry matter percentage, about 90% of plant dry matter percentage, or about 100% of plant dry matter percentage. In some embodiments, total non-structural carbohydrates in a plant is increased by at least about 1% of plant dry matter percentage, about 10% of plant dry matter percentage, about 20% of plant dry matter percentage, about 30% of plant dry matter percentage, about 40% of plant dry matter percentage, about 50% of plant dry matter percentage, about 60% of plant dry matter percentage, about 70% of plant dry matter percentage, about 80% of plant dry matter percentage, about 84% of plant dry matter percentage, or about 90% of plant dry matter percentage. In some embodiments, total non-structural carbohydrates in a plant is increased by at most about 10% of plant dry matter percentage, about 20% of plant dry matter percentage, about 30% of plant dry matter percentage, about 40% of plant dry matter percentage, about 50% of plant dry matter percentage, about 60% of plant dry matter percentage, about 70% of plant dry matter percentage, about 80% of plant dry matter percentage, about 84% of plant dry matter percentage, about 90% of plant dry matter percentage, or about 100% of plant dry matter percentage.

In some embodiments, total non-structural carbohydrates in a plant is increased by about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, to 100%, or greater than 100%. In some embodiments, total non-structural carbohydrates in a plant is increased by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, to 100%, or at least greater than 100%. In some embodiments, total non-structural carbohydrates in a plant is increased by about 1-15, 10-20, 10-30, 10-40, 10-50, 10-60, 10-70, 10-80, 10-90, 10-100, 20-30, 20-40, 20-50, 20-60, 20-70, 20-80, 20-90, 20-100, 30-40, 30-50, 30-60, 30-70, 30-80, 30-90, 30-100, 40-50, 40-60, 40-70, 40-80, 40-90, 40-100, 50-60, 50-70, 50-80, 50-90, 50-100, 60-70, 60-80, 60-90, 60-100, 70-80, 80-90, 90-100, 80-90, 80-100, or 90-100%. In some embodiments, total non-structural carbohydrates in a plant is increased by at least 1-15, 10-20, 10-30, 10-40, 10-50, 10-60, 10-70, 10-80, 10-90, 10-100, 20-30, 20-40, 20-50, 20-60, 20-70, 20-80, 20-90, 20-100, 30-40, 30-50, 30-60, 30-70, 30-80, 30-90, 30-100, 40-50, 40-60, 40-70, 40-80, 40-90, 40-100, 50-60, 50-70, 50-80, 50-90, 50-100, 60-70, 60-80, 60-90, 60-100, 70-80, 80-90, 90-100, 80-90, 80-100, or 90-100%.

In some embodiments, total non-structural carbohydrates in a plant is increased by about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, to 100% of plant dry matter percentage, or greater than 100% of plant dry matter percentage. In some embodiments, total non-structural carbohydrates in a plant is increased by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, to 100% of plant dry matter percentage, or at least greater than 100% of plant dry matter percentage. In some embodiments, total non-structural carbohydrates in a plant is increased by about 1-15, 10-20, 10-30, 10-40, 10-50, 10-60, 10-70, 10-80, 10-90, 10-100, 20-30, 20-40, 20-50, 20-60, 20-70, 20-80, 20-90, 20-100, 30-40, 30-50, 30-60, 30-70, 30-80, 30-90, 30-100, 40-50, 40-60, 40-70, 40-80, 40-90, 40-100, 50-60, 50-70, 50-80, 50-90, 50-100, 60-70, 60-80, 60-90, 60-100, 70-80, 80-90, 90-100, 80-90, 80-100, or 90-100% of plant dry matter percentage. In some embodiments, total non-structural carbohydrates in a plant is increased by at least 1-15, 10-20, 10-30, 10-40, 10-50, 10-60, 10-70, 10-80, 10-90, 10-100, 20-30, 20-40, 20-50, 20-60, 20-70, 20-80, 20-90, 20-100, 30-40, 30-50, 30-60, 30-70, 30-80, 30-90, 30-100, 40-50, 40-60, 40-70, 40-80, 40-90, 40-100, 50-60, 50-70, 50-80, 50-90, 50-100, 60-70, 60-80, 60-90, 60-100, 70-80, 80-90, 90-100, 80-90, 80-100, or 90-100% of plant dry matter percentage.

### 7. Decreasing an Area of Field Required for Feeding Grazing Animals

In some embodiments, treatment of the plant seeds with UV light leads to a decrease in the area of field required for feeding grazing animals following sowing of the treated plant seeds. In some embodiments, an area of field required for feeding grazing animals is decreased by about 1% to about 100%. In some embodiments, an area of field required for feeding grazing animals is decreased by about 1% to about 10%, about 1% to about 20%, about 1% to about 30%, about 1% to about 40%, about 1% to about 50%, about 1% to about 60%, about 1% to about 70%, about 1% to about 80%, about 1% to about 84%, about 1% to about 90%, about 1% to about 100%, about 10% to about 20%, about 10% to about 30%, about 10% to about 40%, about 10% to about 50%, about 10% to about 60%, about 10% to about 70%, about 10% to about 80%, about 10% to about 84%, about 10% to about 90%, about 10% to about 100%, about 20% to about 30%, about 20% to about 40%, about 20% to about 50%, about 20% to about 60%, about 20% to about 70%, about 20% to about 80%, about 20% to about 84%, about 20% to about 90%, about 20% to about 100%, about 30% to about 40%, about 30% to about 50%, about 30% to about 60%, about 30% to about 70%, about 30% to about 80%, about 30% to about 84%, about 30% to about 90%, about 30% to about 100%, about 40% to about 50%, about 40% to about 60%, about 40% to about 70%, about 40% to about 80%, about 40% to about 84%, about 40% to about 90%, about 40% to about 100%, about 50% to about 60%, about 50% to about 70%, about 50% to about 80%, about 50% to about 84%, about 50% to about 90%, about 50% to about 100%, about 60% to about 70%, about 60% to about 80%, about 60% to about 84%, about 60% to about 90%, about 60% to about 100%, about 70% to about 80%, about 70% to about 84%, about 70% to about 90%, about 70% to about 100%, about 80% to about 84%, about 80% to about 90%, about 80% to about 100%, about 84% to about 90%, about 84% to about 100%, or about 90% to about 100%. In some embodiments, an area of field required for feeding grazing animals is decreased by about 1%, about 10%, about 20%, about 30%, about 40%, about 50%, about 60%, about 70%, about 80%, about 84%, about 90%, or about 100%. In some embodiments, an area of field required for feeding grazing animals is decreased by at least about 1%, about 10%, about 20%, about 30%, about 40%, about 50%, about 60%, about 70%, about 80%, about 84%, or about 90%. In some embodiments, an area of field required for feeding grazing animals is decreased by at most about 10%, about 20%, about 30%, about 40%, about 50%, about 60%, about 70%, about 80%, about 84%, about 90%, or about 100%.

In some embodiments, an area of field required for feeding grazing animals is decreased by about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or greater than 100%. In some embodiments, an area of field required for feeding grazing animals is decreased by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or at least greater than 100%. In some embodiments, an area of field required for feeding grazing animals is decreased by about 1-15, 10-20, 10-30, 10-40, 10-50, 10-60, 10-70, 10-80, 10-90, 10-100, 20-30, 20-40, 20-50, 20-60, 20-70, 20-80, 20-90, 20-100, 30-40, 30-50, 30-60, 30-70, 30-80, 30-90, 30-100, 40-50, 40-60, 40-70, 40-80, 40-90, 40-100, 50-60, 50-70, 50-80, 50-90, 50-100, 60-70, 60-80, 60-90, 60-100, 70-80, 80-90, 90-100, 80-90, 80-100, or 90-100%. In some embodiments, an area of field required for feeding grazing animals is decreased by at least 1-15, 10-20, 10-30, 10-40, 10-50, 10-60, 10-70, 10-80, 10-90, 10-100, 20-30, 20-40, 20-50, 20-60, 20-70, 20-80, 20-90, 20-100, 30-40, 30-50, 30-60, 30-70, 30-80, 30-90, 30-100, 40-50, 40-60, 40-70, 40-80, 40-90, 40-100, 50-60, 50-70, 50-80, 50-90, 50-100, 60-70, 60-80, 60-90, 60-100, 70-80, 80-90, 90-100, 80-90, 80-100, or 90-100%.

### 8. Reducing the Methane Production of Grazing Animals

In some embodiments, treatment of the plant seeds with UV light leads to a decrease in methane production of grazing animals that eat plants grown from the UV treated seeds. In some embodiments, the methane production of grazing animals is decreased by about 1% to about 100%. In some embodiments, the methane production of grazing animals is decreased by about 1% to about 10%, about 1% to about 20%, about 1% to about 30%, about 1% to about 40%, about 1% to about 50%, about 1% to about 60%, about 1% to about 70%, about 1% to about 80%, about 1% to about 84%, about 1% to about 90%, about 1% to about 100%, about 10% to about 20%, about 10% to about 30%, about 10% to about 40%, about 10% to about 50%, about 10% to about 60%, about 10% to about 70%, about 10% to about 80%, about 10% to about 84%, about 10% to about 90%, about 10% to about 100%, about 20% to about 30%, about 20% to about 40%, about 20% to about 50%, about 20% to about 60%, about 20% to about 70%, about 20% to about 80%, about 20% to about 84%, about 20% to about 90%, about 20% to about 100%, about 30% to about 40%, about 30% to about 50%, about 30% to about 60%, about 30% to about 70%, about 30% to about 80%, about 30% to about 84%, about 30% to about 90%, about 30% to about 100%, about 40% to about 50%, about 40% to about 60%, about 40% to about 70%, about 40% to about 80%, about 40% to about 84%, about 40% to about 90%, about 40% to about 100%, about 50% to about 60%, about 50% to about 70%, about 50% to about 80%, about 50% to about 84%, about 50% to about 90%, about 50% to about 100%, about 60% to about 70%, about 60% to about 80%, about 60% to about 84%, about 60% to about 90%, about 60% to about 100%, about 70% to about 80%, about 70% to about 84%, about 70% to about 90%, about 70% to about 100%, about 80% to about 84%, about 80% to about 90%, about 80% to about 100%, about 84% to about 90%, about 84% to about 100%, or about 90% to about 100%. In some embodiments, the methane production of grazing animals is decreased by about 1%, about 10%, about 20%, about 30%, about 40%, about 50%, about 60%, about 70%, about 80%, about 84%, about 90%, or about 100%. In some embodiments, the methane production of grazing animals is decreased by at least about 1%, about 10%, about 20%, about 30%, about 40%, about 50%, about 60%, about 70%, about 80%, about 84%, or about 90%. In some embodiments, the methane production of grazing animals is decreased by at most about 10%, about 20%, about 30%, about 40%, about 50%, about 60%, about 70%, about 80%, about 84%, about 90%, or about 100%.

In some embodiments, the methane production of grazing animals is decreased by about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or greater than 100%. In some embodiments, the methane production of grazing animals is decreased by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or at least greater than 100%. In some embodiments, the methane production of grazing animals is decreased by about 1-15, 10-20, 10-30, 10-40, 10-50, 10-60, 10-70, 10-80, 10-90, 10-100, 20-30, 20-40, 20-50, 20-60, 20-70, 20-80, 20-90, 20-100, 30-40, 30-50, 30-60, 30-70, 30-80, 30-90, 30-100, 40-50, 40-60, 40-70, 40-80, 40-90, 40-100, 50-60, 50-70, 50-80, 50-90, 50-100, 60-70, 60-80, 60-90, 60-100, 70-80, 80-90, 90-100, 80-90, 80-100, or 90-100%. In some embodiments, the methane production of grazing animals is decreased by at least 1-15, 10-20, 10-30, 10-40, 10-50, 10-60, 10-70, 10-80, 10-90, 10-100, 20-30, 20-40, 20-50, 20-60, 20-70, 20-80, 20-90, 20-100, 30-40, 30-50, 30-60, 30-70, 30-80, 30-90, 30-100, 40-50, 40-60, 40-70, 40-80, 40-90, 40-100, 50-60, 50-70, 50-80, 50-90, 50-100, 60-70, 60-80, 60-90, 60-100, 70-80, 80-90, 90-100, 80-90, 80-100, or 90-100%.

9. Sequestering Carbon from the Atmosphere

In some embodiments, treatment of the plant seeds with UV light leads to an increase in the amount of carbon sequestered from the atmosphere by plants grown from UV treated seeds. In some embodiments, the amount of carbon sequestered from the atmosphere is increased by about 1% to about 100%. In some embodiments, the amount of carbon sequestered from the atmosphere is increased by about 1% to about 10%, about 1% to about 20%, about 1% to about 30%, about 1% to about 40%, about 1% to about 50%, about 1% to about 60%, about 1% to about 70%, about 1% to about 80%, about 1% to about 84%, about 1% to about 90%, about 1% to about 100%, about 10% to about 20%, about 10% to about 30%, about 10% to about 40%, about 10% to about 50%, about 10% to about 60%, about 10% to about 70%, about 10% to about 80%, about 10% to about 84%, about 10% to about 90%, about 10% to about 100%, about 20% to about 30%, about 20% to about 40%, about 20% to about 50%, about 20% to about 60%, about 20% to about 70%, about 20% to about 80%, about 20% to about 84%, about 20% to about 90%, about 20% to about 100%, about 30% to about 40%, about 30% to about 50%, about 30% to about 60%, about 30% to about 70%, about 30% to about 80%, about 30% to about 84%, about 30% to about 90%, about 30% to about 100%, about 40% to about 50%, about 40% to about 60%, about 40% to about 70%, about 40% to about 80%, about 40% to about 84%, about 40% to about 90%, about 40% to about 100%, about 50% to about 60%, about 50% to about 70%, about 50% to about 80%, about 50% to about 84%, about 50% to about 90%, about 50% to about 100%, about 60% to about 70%, about 60% to about 80%, about 60% to about 84%, about 60% to about 90%, about 60% to about 100%, about 70% to about 80%, about 70% to about 84%, about 70% to about 90%, about 70% to about 100%, about 80% to about 84%, about 80% to about 90%, about 80% to about 100%, about 84% to about 90%, about 84% to about 100%, or about 90% to about 100%. In some embodiments, the amount of carbon sequestered from the atmosphere is increased by about 1%, about 10%, about 20%, about 30%, about 40%, about 50%, about 60%, about 70%, about 80%, about 84%, about 90%, or about 100%. In some embodiments, the amount of carbon sequestered from the atmosphere is increased by at least about 1%, about 10%, about 20%, about 30%, about 40%, about 50%, about 60%, about 70%, about 80%, about 84%, or about 90%. In some embodiments, the amount of carbon sequestered from the atmosphere is increased by at most about 10%, about 20%, about 30%, about 40%, about 50%, about 60%, about 70%, about 80%, about 84%, about 90%, or about 100%.

In some embodiments, an amount of carbon sequestered from the atmosphere is increased by about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or greater than 100%. In some embodiments, an amount of carbon sequestered from the atmosphere is increased by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or at least greater than 100%. In some embodiments, an amount of carbon sequestered from the atmosphere is increased by about 1-15, 10-20, 10-30, 10-40, 10-50, 10-60, 10-70, 10-80, 10-90, 10-100, 20-30, 20-40, 20-50, 20-60, 20-70, 20-80, 20-90, 20-100, 30-40, 30-50, 30-60, 30-70, 30-80, 30-90, 30-100, 40-50, 40-60, 40-70, 40-80, 40-90, 40-100, 50-60, 50-70, 50-80, 50-90, 50-100, 60-70, 60-80, 60-90, 60-100, 70-80, 80-90, 90-100, 80-90, 80-100, or 90-100%. In some embodiments, an amount of carbon sequestered from the atmosphere is increased by at least 1-15, 10-20, 10-30, 10-40, 10-50, 10-60, 10-70, 10-80, 10-90, 10-100, 20-30, 20-40, 20-50, 20-60, 20-70, 20-80, 20-90, 20-100, 30-40, 30-50, 30-60, 30-70, 30-80, 30-90, 30-100, 40-50, 40-60, 40-70, 40-80, 40-90, 40-100, 50-60, 50-70, 50-80, 50-90, 50-100, 60-70, 60-80, 60-90, 60-100, 70-80, 80-90, 90-100, 80-90, 80-100, or 90-100%.

### 10. Increasing Crude Fat Concentration in a Plant

In some embodiments, treatment of the plant seeds with UV light leads to an increase in the amount of crude fat concentration in a plant grown from UV treated seeds.

In some embodiments, the amount of crude fat concentration in a plant is increased by about 1% to about 12%. In some embodiments, the amount of crude fat concentration in a plant is increased by about 1% to about 2%, about 1% to about 3%, about 1% to about 4%, about 1% to about 5%, about 1% to about 6%, about 1% to about 7%, about 1% to about 8%, about 1% to about 9%, about 1% to about 10%, about 1% to about 11%, about 1% to about 12%, about 2% to about 3%, about 2% to about 4%, about 2% to about 5%, about 2% to about 6%, about 2% to about 7%, about 2% to about 8%, about 2% to about 9%, about 2% to about 10%, about 2% to about 11%, about 2% to about 12%, about 3% to about 4%, about 3% to about 5%, about 3% to about 6%, about 3% to about 7%, about 3% to about 8%, about 3% to about 9%, about 3% to about 10%, about 3% to about 11%, about 3% to about 12%, about 4% to about 5%, about 4% to about 6%, about 4% to about 7%, about 4% to about 8%, about 4% to about 9%, about 4% to about 10%, about 4% to about 11%, about 4% to about 12%, about 5% to about 6%, about 5% to about 7%, about 5% to about 8%, about 5% to about 9%, about 5% to about 10%, about 5% to about 11%, about 5% to about 12%, about 6% to about 7%, about 6% to about 8%, about 6% to about 9%, about 6% to about 10%, about 6% to about 11%, about 6% to about 12%, about 7% to about 8%, about 7% to about 9%, about 7% to about 10%, about 7% to about 11%, about 7% to about 12%, about 8% to about 9%, about 8% to about 10%, about 8% to about 11%, about 8% to about 12%, about 9% to about 10%, about 9% to about 11%, about 9% to about 12%, about 10% to about 11%, about 10% to about 12%, or about 11% to about 12%. In some embodiments, the amount of crude fat concentration in a plant is increased by about 1%, about 2%, about 3%, about 4%, about 5%, about 6%, about 7%, about 8%, about 9%, about 10%, about 11%, or about 12%. In some embodiments, the amount of crude fat concentration in a plant is increased by at least about 1%, about 2%, about 3%, about 4%, about 5%, about 6%, about 7%, about 8%, about 9%, about 10%, or about 11%. In some embodiments, the amount of crude fat concentration in a plant is increased by at most about 2%, about 3%, about 4%, about 5%, about 6%, about 7%, about 8%, about 9%, about 10%, about 11%, or about 12%.

In some embodiments, the amount of crude fat concentration in a plant is increased by about 1% to about 15%. In some embodiments, the amount of crude fat concentration in a plant is increased by about 1% to about 5%, about 1% to about 9.4%, about 1% to about 10%, about 1% to about 15%, about 5% to about 9.4%, about 5% to about 10%, about 5% to about 15%, about 9.4% to about 10%, about 9.4% to about 15%, or about 10% to about 15%. In some embodiments, the amount of crude fat concentration in a plant is increased by about 1%, about 5%, about 9.4%, about 10%, or about 15%. In some embodiments, the amount of crude fat concentration in a plant is increased by at least about 1%, about 5%, about 9.4%, or about 10%. In some embodiments, the amount of crude fat concentration in a plant is increased by at most about 5%, about 9.4%, about 10%, or about 15%.

In some embodiments, an amount of crude fat concentration in a plant is increased by about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or greater than 100%. In some embodiments, an amount of crude fat concentration in a plant is increased by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or at least greater than 100%. In some embodiments, an amount of crude fat concentration in a plant is increased by about 1-15, 10-20, 10-30, 10-40, 10-50, 10-60, 10-70, 10-80, 10-90, 10-100, 20-30, 20-40, 20-50, 20-60, 20-70, 20-80, 20-90, 20-100, 30-40, 30-50, 30-60, 30-70, 30-80, 30-90, 30-100, 40-50, 40-60, 40-70, 40-80, 40-90, 40-100, 50-60, 50-70, 50-80, 50-90, 50-100, 60-70, 60-80, 60-90, 60-100, 70-80, 80-90, 90-100, 80-90, 80-100, or 90-100%. In some embodiments, an amount of crude fat concentration in a plant is increased by at least 10-20, 10-30, 10-40, 10-50, 10-60, 10-70, 10-80, 10-90, 10-100, 20-30, 20-40, 20-50, 20-60, 20-70, 20-80, 20-90, 20-100, 30-40, 30-50, 30-60, 30-70, 30-80, 30-90, 30-100, 40-50, 40-60, 40-70, 40-80, 40-90, 40-100, 50-60, 50-70, 50-80, 50-90, 50-100, 60-70, 60-80, 60-90, 60-100, 70-80, 80-90, 90-100, 80-90, 80-100, or 90-100%.

### 11. Forage Biomass of the Plant

In some embodiments, treatment of the plant seeds with UV light leads to an increase in the amount of forage biomass in a plant grown from UV treated seeds. In some embodiments, the amount of forage biomass of a plant is increased by about 1% to about 55%. In some embodiments, the amount of forage biomass of a plant is increased by about 1% to about 10%, about 1% to about 15%, about 1% to about 20%, about 1% to about 25%, about 1% to about 30%, about 1% to about 35%, about 1% to about 37%, about 1% to about 40%, about 1% to about 45%, about 1% to about 50%, about 1% to about 55%, about 10% to about 15%, about 10% to about 20%, about 10% to about 25%, about 10% to about 30%, about 10% to about 35%, about 10% to about 37%, about 10% to about 40%, about 10% to about 45%, about 10% to about 50%, about 10% to about 55%, about 15% to about 20%, about 15% to about 25%, about 15% to about 30%, about 15% to about 35%, about 15% to about 37%, about 15% to about 40%, about 15% to about 45%, about 15% to about 50%, about 15% to about 55%, about 20% to about 25%, about 20% to about 30%, about 20% to about 35%, about 20% to about 37%, about 20% to about 40%, about 20% to about 45%, about 20% to about 50%, about 20% to about 55%, about 25% to about 30%, about 25% to about 35%, about 25% to about 37%, about 25% to about 40%, about 25% to about 45%, about 25% to about 50%, about 25% to about 55%, about 30% to about 35%, about 30% to about 37%, about 30% to about 40%, about 30% to about 45%, about 30% to about 50%, about 30% to about 55%, about 35% to about 37%, about 35% to about 40%, about 35% to about 45%, about 35% to about 50%, about 35% to about 55%, about 37% to about 40%, about 37% to about 45%, about 37% to about 50%, about 37% to about 55%, about 40% to about 45%, about 40% to about 50%, about 40% to about 55%, about 45% to about 50%, about 45% to about 55%, or about 50% to about 55%. In some embodiments, the amount of forage biomass of a plant is increased by about 1%, about 10%, about 15%, about 20%, about 25%, about 30%, about 35%, about 37%, about 40%, about 45%, about 50%, or about 55%. In some embodiments, the amount of forage biomass of a plant is increased by at least about 1%, about 10%, about 15%, about 20%, about 25%, about 30%, about 35%, about 37%, about 40%, about 45%, or about 50%. In some embodiments, the amount of forage biomass of a plant is increased by at most about 10%, about 15%, about 20%, about 25%, about 30%, about 35%, about 37%, about 40%, about 45%, about 50%, or about 55%.

In some embodiments, an amount of forage biomass of a plant is increased by about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or greater than 100%. In some embodiments, an amount of forage biomass of a plant is increased by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or at least greater than 100%. In some embodiments, an amount of forage biomass of a plant is increased by about 1-15, 10-20, 10-30, 10-40, 10-50, 10-60, 10-70, 10-80, 10-90, 10-100, 20-30, 20-40, 20-50, 20-60, 20-70, 20-80, 20-90, 20-100, 30-40, 30-50, 30-60, 30-70, 30-80, 30-90, 30-100, 40-50, 40-60, 40-70, 40-80, 40-90, 40-100, 50-60, 50-70, 50-80, 50-90, 50-100, 60-70, 60-80, 60-90, 60-100, 70-80, 80-90, 90-100, 80-90, 80-100, or 90-100%. In some embodiments, an amount of forage biomass of a plant is increased by at least 10-20, 10-30, 10-40, 10-50, 10-60, 10-70, 10-80, 10-90, 10-100, 20-30, 20-40, 20-50, 20-60, 20-70, 20-80, 20-90, 20-100, 30-40, 30-50, 30-60, 30-70, 30-80, 30-90, 30-100, 40-50, 40-60, 40-70, 40-80, 40-90, 40-100, 50-60, 50-70, 50-80, 50-90, 50-100, 60-70, 60-80, 60-90, 60-100, 70-80, 80-90, 90-100, 80-90, 80-100, or 90-100%.

### 12. Tiller Number

In some embodiments, treatment of the plant seeds with UV light leads to an increase in the tiller number of a plant grown from UV treated seeds. In some embodiments, the tiller number of a plant is increased by about 1% to about 50%. In some embodiments, the tiller number of a plant is increased by about 1% to about 10%, about 1% to about 15%, about 1% to about 20%, about 1% to about 21%, about 1% to about 25%, about 1% to about 30%, about 1% to about 35%, about 1% to about 40%, about 1% to about 45%, about 1% to about 50%, about 10% to about 15%, about 10% to about 20%, about 10% to about 21%, about 10% to about 25%, about 10% to about 30%, about 10% to about 35%, about 10% to about 40%, about 10% to about 45%, about 10% to about 50%, about 15% to about 20%, about 15% to about 21%, about 15% to about 25%, about 15% to about 30%, about 15% to about 35%, about 15% to about 40%, about 15% to about 45%, about 15% to about 50%, about 20% to about 21%, about 20% to about 25%, about 20% to about 30%, about 20% to about 35%, about 20% to about 40%, about 20% to about 45%, about 20% to about 50%, about 21% to about 25%, about 21% to about 30%, about 21% to about 35%, about 21% to about 40%, about 21% to about 45%, about 21% to about 50%, about 25% to about 30%, about 25% to about 35%, about 25% to about 40%, about 25% to about 45%, about 25% to about 50%, about 30% to about 35%, about 30% to about 40%, about 30% to about 45%, about 30% to about 50%, about 35% to about 40%, about 35% to about 45%, about 35% to about 50%, about 40% to about 45%, about 40% to about 50%, or about 45% to about 50%. In some embodiments, the tiller number of a plant is increased by about 1%, about 10%, about 15%, about 20%, about 21%, about 25%, about 30%, about 35%, about 40%, about 45%, or about 50%. In some embodiments, the tiller number of a plant is increased by at least about 1%, about 10%, about 15%, about 20%, about 21%, about 25%, about 30%, about 35%, about 40%, or about 45%. In some embodiments, the tiller number of a plant is increased by at most about 10%, about 15%, about 20%, about 21%, about 25%, about 30%, about 35%, about 40%, about 45%, or about 50%.

In some embodiments, a tiller number of a plant is increased by about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or greater than 100%. In some embodiments, a tiller number of a plant is increased by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or at least greater than 100%. In some embodiments, a tiller number of a plant is increased by about 1-15, 10-20, 10-30, 10-40, 10-50, 10-60, 10-70, 10-80, 10-90, 10-100, 20-30, 20-40, 20-50, 20-60, 20-70, 20-80, 20-90, 20-100, 30-40, 30-50, 30-60, 30-70, 30-80, 30-90, 30-100, 40-50, 40-60, 40-70, 40-80, 40-90, 40-100, 50-60, 50-70, 50-80, 50-90, 50-100, 60-70, 60-80, 60-90, 60-100, 70-80, 80-90, 90-100, 80-90, 80-100, or 90-100%. In some embodiments, a tiller number of a plant is increased by at least 1-15, 10-20, 10-30, 10-40, 10-50, 10-60, 10-70, 10-80, 10-90, 10-100, 20-30, 20-40, 20-50, 20-60, 20-70, 20-80, 20-90, 20-100, 30-40, 30-50, 30-60, 30-70, 30-80, 30-90, 30-100, 40-50, 40-60, 40-70, 40-80, 40-90, 40-100, 50-60, 50-70, 50-80, 50-90, 50-100, 60-70, 60-80, 60-90, 60-100, 70-80, 80-90, 90-100, 80-90, 80-100, or 90-100%.

### 13. Shoot Fresh Weight of the Grass

In some embodiments, treatment of the plant seeds with UV light leads to an increase in the shoot fresh weight of a plant grown from UV treated seeds. In some embodiments, the shoot fresh weight of a plant is increased by about 1% to about 50%. In some embodiments, the shoot fresh weight of a plant is increased by about 1% to about 10%, about 1% to about 15%, about 1% to about 18%, about 1% to about 20%, about 1% to about 25%, about 1% to about 30%, about 1% to about 35%, about 1% to about 37%, about 1% to about 40%, about 1% to about 45%, about 1% to about 50%, about 10% to about 15%, about 10% to about 18%, about 10% to about 20%, about 10% to about 25%, about 10% to about 30%, about 10% to about 35%, about 10% to about 37%, about 10% to about 40%, about 10% to about 45%, about 10% to about 50%, about 15% to about 18%, about 15% to about 20%, about 15% to about 25%, about 15% to about 30%, about 15% to about 35%, about 15% to about 37%, about 15% to about 40%, about 15% to about 45%, about 15% to about 50%, about 18% to about 20%, about 18% to about 25%, about 18% to about 30%, about 18% to about 35%, about 18% to about 37%, about 18% to about 40%, about 18% to about 45%, about 18% to about 50%, about 20% to about 25%, about 20% to about 30%, about 20% to about 35%, about 20% to about 37%, about 20% to about 40%, about 20% to about 45%, about 20% to about 50%, about 25% to about 30%, about 25% to about 35%, about 25% to about 37%, about 25% to about 40%, about 25% to about 45%, about 25% to about 50%, about 30% to about 35%, about 30% to about 37%, about 30% to about 40%, about 30% to about 45%, about 30% to about 50%, about 35% to about 37%, about 35% to about 40%, about 35% to about 45%, about 35% to about 50%, about 37% to about 40%, about 37% to about 45%, about 37% to about 50%, about 40% to about 45%, about 40% to about 50%, or about 45% to about 50%. In some embodiments, the shoot fresh weight of a plant is increased by about 1%, about 10%, about 15%, about 18%, about 20%, about 25%, about 30%, about 35%, about 37%, about 40%, about 45%, or about 50%. In some embodiments, the shoot fresh weight of a plant is increased by at least about 1%, about 10%, about 15%, about 18%, about 20%, about 25%, about 30%, about 35%, about 37%, about 40%, or about 45%. In some embodiments, the shoot fresh weight of a plant is increased by at most about 10%, about 15%, about 18%, about 20%, about 25%, about 30%, about 35%, about 37%, about 40%, about 45%, or about 50%.

In some embodiments, the shoot fresh weight of a plant is increased by about 0.5 g to about 6 g. In some embodiments, the shoot fresh weight of a plant is increased by about 0.5 g to about 1 g, about 0.5 g to about 1.5 g, about 0.5 g to about 2 g, about 0.5 g to about 2.5 g, about 0.5 g to about 3 g, about 0.5 g to about 3.5 g, about 0.5 g to about 4 g, about 0.5 g to about 4.5 g, about 0.5 g to about 5 g, about 0.5 g to about 5.5 g, about 0.5 g to about 6 g, about 1 g to about 1.5 g, about 1 g to about 2 g, about 1 g to about 2.5 g, about 1 g to about 3 g, about 1 g to about 3.5 g, about 1 g to about 4 g, about 1 g to about 4.5 g, about 1 g to about 5 g, about 1 g to about 5.5 g, about 1 g to about 6 g, about 1.5 g to about 2 g, about 1.5 g to about 2.5 g, about 1.5 g to about 3 g, about 1.5 g to about 3.5 g, about 1.5 g to about 4 g, about 1.5 g to about 4.5 g, about 1.5 g to about 5 g, about 1.5 g to about 5.5 g, about 1.5 g to about 6 g, about 2 g to about 2.5 g, about 2 g to about 3 g, about 2 g to about 3.5 g, about 2 g to about 4 g, about 2 g to about 4.5 g, about 2 g to about 5 g, about 2 g to about 5.5 g, about 2 g to about 6 g, about 2.5 g to about 3 g, about 2.5 g to about 3.5 g, about 2.5 g to about 4 g, about 2.5 g to about 4.5 g, about 2.5 g to about 5 g, about 2.5 g to about 5.5 g, about 2.5 g to about 6 g, about 3 g to about 3.5 g, about 3 g to about 4 g, about 3 g to about 4.5 g, about 3 g to about 5 g, about 3 g to about 5.5 g, about 3 g to about 6 g, about 3.5 g to about 4 g, about 3.5 g to about 4.5 g, about 3.5 g to about 5 g, about 3.5 g to about 5.5 g, about 3.5 g to about 6 g, about 4 g to about 4.5 g, about 4 g to about 5 g, about 4 g to about 5.5 g, about 4 g to about 6 g, about 4.5 g to about 5 g, about 4.5 g to about 5.5 g, about 4.5 g to about 6 g, about 5 g to about 5.5 g, about 5 g to about 6 g, or about 5.5 g to about 6 g. In some embodiments, the shoot fresh weight of a plant is increased by about 0.5 g, about 1 g, about 1.5 g, about 2 g, about 2.5 g, about 3 g, about 3.5 g, about 4 g, about 4.5 g, about 5 g, about 5.5 g, or about 6 g. In some embodiments, the shoot fresh weight of a plant is increased by at least about 0.5 g, about 1 g, about 1.5 g, about 2 g, about 2.5 g, about 3 g, about 3.5 g, about 4 g, about 4.5 g, about 5 g, or about 5.5 g. In some embodiments, the shoot fresh weight of a plant is increased by at most about 1 g, about 1.5 g, about 2 g, about 2.5 g, about 3 g, about 3.5 g, about 4 g, about 4.5 g, about 5 g, about 5.5 g, or about 6 g.

In some embodiments, a shoot fresh weight of a plant is increased by about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or greater than 100%. In some embodiments, a shoot fresh weight of a plant is increased by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or at least greater than 100%. In some embodiments, a shoot fresh weight of a plant is increased by about 1-15, 10-20, 10-30, 10-40, 10-50, 10-60, 10-70, 10-80, 10-90, 10-100, 20-30, 20-40, 20-50, 20-60, 20-70, 20-80, 20-90, 20-100, 30-40, 30-50, 30-60, 30-70, 30-80, 30-90, 30-100, 40-50, 40-60, 40-70, 40-80, 40-90, 40-100, 50-60, 50-70, 50-80, 50-90, 50-100, 60-70, 60-80, 60-90, 60-100, 70-80, 80-90, 90-100, 80-90, 80-100, or 90-100%. In some embodiments, a shoot fresh weight of a plant is increased by at least 1-15, 10-20, 10-30, 10-40, 10-50, 10-60, 10-70, 10-80, 10-90, 10-100, 20-30, 20-40, 20-50, 20-60, 20-70, 20-80, 20-90, 20-100, 30-40, 30-50, 30-60, 30-70, 30-80, 30-90, 30-100, 40-50, 40-60, 40-70, 40-80, 40-90, 40-100, 50-60, 50-70, 50-80, 50-90, 50-100, 60-70, 60-80, 60-90, 60-100, 70-80, 80-90, 90-100, 80-90, 80-100, or 90-100%.

### 14. Gene Expression

In some embodiments, treatment of the plant seeds with UV light leads to an increase or decrease in gene expression of a gene in a plant grown from UV treated seeds. In some embodiments, expression of a gene is increased or decreased by about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or greater than 100%. In some embodiments, expression of a gene is increased or decreased by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or at least greater than 100%. In some embodiments, expression of a gene is increased or decreased by about 1-15, 10-20, 10-30, 10-40, 10-50, 10-60, 10-70, 10-80, 10-90, 10-100, 20-30, 20-40, 20-50, 20-60, 20-70, 20-80, 20-90, 20-100, 30-40, 30-50, 30-60, 30-70, 30-80, 30-90, 30-100, 40-50, 40-60, 40-70, 40-80, 40-90, 40-100, 50-60, 50-70, 50-80, 50-90, 50-100, 60-70, 60-80, 60-90, 60-100, 70-80, 80-90, 90-100, 80-90, 80-100, or 90-100%. In some embodiments, expression of a gene is increased or decreased by at least 1-15, 10-20, 10-30, 10-40, 10-50, 10-60, 10-70, 10-80, 10-90, 10-100, 20-30, 20-40, 20-50, 20-60, 20-70, 20-80, 20-90, 20-100, 30-40, 30-50, 30-60, 30-70, 30-80, 30-90, 30-100, 40-50, 40-60, 40-70, 40-80, 40-90, 40-100, 50-60, 50-70, 50-80, 50-90, 50-100, 60-70, 60-80, 60-90, 60-100, 70-80, 80-90, 90-100, 80-90, 80-100, or 90-100%.

In some embodiments, expression of OLE or DGAT is increased or decreased by about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or greater than 100%. In some embodiments, expression of OLE or DGAT is increased or decreased by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or at least greater than 100%. In some embodiments, expression of OLE or DGAT is increased or decreased by about 1-15, 10-20, 10-30, 10-40, 10-50, 10-60, 10-70, 10-80, 10-90, 10-100, 20-30, 20-40, 20-50, 20-60, 20-70, 20-80, 20-90, 20-100, 30-40, 30-50, 30-60, 30-70, 30-80, 30-90, 30-100, 40-50, 40-60, 40-70, 40-80, 40-90, 40-100, 50-60, 50-70, 50-80, 50-90, 50-100, 60-70, 60-80, 60-90, 60-100, 70-80, 80-90, 90-100, 80-90, 80-100, or 90-100%. In some embodiments, expression of OLE or DGAT is increased or decreased by at least 1-15, 10-20, 10-30, 10-40, 10-50, 10-60, 10-70, 10-80, 10-90, 10-100, 20-30, 20-40, 20-50, 20-60, 20-70, 20-80, 20-90, 20-100, 30-40, 30-50, 30-60, 30-70, 30-80, 30-90, 30-100, 40-50, 40-60, 40-70, 40-80, 40-90, 40-100, 50-60, 50-70, 50-80, 50-90, 50-100, 60-70, 60-80, 60-90, 60-100, 70-80, 80-90, 90-100, 80-90, 80-100, or 90-100%.

### D. Plant Performance Continued

In certain aspects, the methods described herein comprise methods of increasing plant performance as described herein, including increasing the lipid content or concentration of a plant as described herein.

### 1. Lipids

Lipid content in grasses are typically low compared to other feed components. However, lipids still contribute to the nutritional profile of the feed. In some embodiments, lipids comprise triacylglycerols, phospholipids, sterols, galactolipids, fatty acids, and tocopherols. In some embodiments, the lipid comprises a triacylglycerol. In some embodiments, the lipid comprises a phospholipid. In some embodiments, the phospholipid comprises phosphatidylcholine or phosphatidylethanolamine. In some embodiments, the lipid comprises a sterol. In some embodiments, the sterol comprises sitosterol, campesterol, or stigmasterol. In some embodiments, the lipid comprises a galactolipid. In some embodiments, the galactolipid comprises digalactosyldiacylglycerol or monogalactosyldiacylglycerol. In some embodiments, the lipid comprises a fatty acid. In some embodiments, the fatty acid comprises palmitic acid, palmitoleic acid, stearic acid, oleic acid, linoleic acid, linolenic acid, arachidic acid, gondoic acid, behenic acid, or erucic acid. In some embodiments, the lipid comprises a tocopherol. In some embodiments, the tocopherol comprises Vitamin E.

In some embodiments, the total lipid percent is measured by gas chromatography. In some embodiments, the total lipid percent is measured by near infrared spectroscopy.

### 2. Additional Metrics of Plant Performance

Plant performance in plants treated using methods as described herein may result in improved plant performance as compared to the counterpart plant material that has not been treated. In some instances, hardiness of the plant material is improved using methods as described herein. In some instances, plant performance comprises at least one of flavonoid levels, anthocyanin levels, size, dry weight, nitrogen index, shoot dry weight, shoot fresh weight, shoot length, radical length, pigment production, leaf size, hypocotyl length, chlorophyll level, leaf area, and root dry weight. In some instances, plant performance comprise plant performance. In some improvements, an improvement in plant performance is an improvement in lipid content.

In some instances, plant performance is determined by determining at least one of fruit fresh weight, number of fruit harvested, cannabinoid concentration, Brix content, fruit width, fruit length, leaf size, leaf surface area, dry weight, nitrogen content, shoot dry weight, shoot fresh weight, root dry weight, vegetable development, flower plant performance per plants, plant performance of fruiting parts, weight of fruiting parts, hardiness, and seed germination rate. In some instances, plant performance is fruit fresh weight. In some instances, plant performance is Brix content. In some instances, plant performance is number of fruit harvested. In some instances, plant performance is branching. In some instances, plant performance is number of flowering parts. In some instances, plant performance is the flower plant performance per plant. In some instances, plant performance is the cannabinoid concentration. In some embodiments, the improved plant performance is an improvement in cannabinoid concentration or cannabinoid composition. In some embodiments, the improved plant performance is an improvement in terpene concentration or terpene profile.

In some instances, methods and devices as described herein result in improved plant performance as determined by an increase in number of fruit harvested. In some instances, methods and devices described herein result in at least or about 3000, 4000, 5000, 6000, 7000, 8000, 9000, 10000, 12000, 14000, 16000, 18000, 20000, 24000, 28000, 32000, 36000, 40000, 50000, 60000, 80000, 100000, or more than 100000 pounds of fruit harvested per acre. In some instances, methods and devices described herein result in a range of about 3000 to about 100000, about 4000 to about 80000, about 6000 to about 60000, about 10000 to about 40000, or about 20000 to about 30000 pounds of fruit harvested per acre. In some instances, methods and devices described herein result in more than 50000 pounds harvested per acre. In some instances, methods and devices described herein result in at least or about 3000, 4000, 5000, 6000, 7000, 8000, 9000, 10000, 12000, 14000, 16000, 18000, 20000, 24000, 28000, 32000, or more than 32000 trays per acre. In some instances, each try comprises about 9.5 pounds to about 10 pounds.

In some embodiments, plant performance is determined by measuring bushels of plant per acre (bu/ac).

In some embodiments, plants exposed to a method as disclosed herein result in an increase of Bu/ac of at least or about 1-100%. In some embodiments, plants exposed to a method as disclosed herein result in an increase of Bu/ac of at least or about 5%, 10%, 15%, 20%, 25%, 30%, 35%, 40%, 45%, 50%, 55%, 60%, 65%, 70%, 75%, 80%, 85%, 90%, 95%, 100% or greater than 100%. In some embodiments, plants exposed to a method as disclosed herein result in an increase of Bu/ac of at least or about 1-1000, 1-900, 1-800, 1-700, 1-600, 1-500, 1-400, 1-300, 1-200, 1-100, 1-50, 1-25, 10-150, 30-100, or 1-10 Bu/ac.

In some embodiments, grass exposed to a method as disclosed herein result in an increase of Bu/ac of at least or about 1-100%. In some embodiments, grass exposed to a method as disclosed herein result in an increase of Bu/ac of at least or about 5%, 10%, 15%, 20%, 25%, 30%, 35%, 40%, 45%, 50%, 55%, 60%, 65%, 70%, 75%, 80%, 85%, 90%, 95%, 100% or greater than 100%. In some embodiments, grass exposed to a method as disclosed herein result in an increase of Bu/ac of at least or about 1-1000, 1-900, 1-800, 1-700, 1-600, 1-500, 1-400, 1-300, 1-200, 1-100, 1-50, 1-25, 10-150, 30-100, or 1-10 Bu/ac.

In some embodiments, corn exposed to a method as disclosed herein result in an increase of Bu/ac of at least or about 1-100%. In some embodiments, corn exposed to a method as disclosed herein result in an increase of Bu/ac of at least or about 5%, 10%, 15%, 20%, 25%, 30%, 35%, 40%, 45%, 50%, 55%, 60%, 65%, 70%, 75%, 80%, 85%, 90%, 95%, 100% or greater than 100%. In some embodiments, corn exposed to a method as disclosed herein result in an increase of Bu/ac of at least or about 1-1000, 1-900, 1-800, 1-700, 1-600, 1-500, 1-400, 1-300, 1-200, 1-100, 1-50, 1-25, 10-150, 30-100, or 1-10 Bu/ac.

In some instances, methods and devices as described herein result in improved plant performance as determined by an increase in average fruit biomass. In some instances, methods and devices described herein result in at least or about 0.1, 0.2, 0.25, 0.3, 0.4, 0.5, 0.6, 0.7, 0.8, 0.9, 1.0, 1.25, 1.5, 1.75, 2.0, 2.25, 2.5, 2.75, 3.0, 3.5, 4.0, 5.0, or more than 5.0 pounds of fruit per foot of row. In some instances, methods and devices described herein result in a range of about 0.1 to about 5.0, about 0.2 to about 4.0, about 0.3 to about 3.5, about 0.4 to about 3.0, about 0.5 to about 2.5, or about 0.75 to about 2 pounds of fruit per foot of row. In some instances, the fruit is tomato, strawberry, or cannabis. In some instances, the fruit is strawberry.

In some instances, plant performance as determined by a metric as disclosed herein is improved by a significant percentage when compared to a counterpart plant that has not been irradiated with a UV-B regimen disclosed herein. Plant performance as determined by a metric as disclosed herein may be improved by about 5%-100%, 10%-90%, 20%-80%, 30%-70%, 40%-60%, 50%-95%, 65%-85%, or 75%-95%. Plant performance as determined by a metric as disclosed herein may be improved by at least about 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 99%, or 100%. Plant performance as determined by a metric as disclosed herein may be improved by at least or about 5%. Plant performance as determined by a metric as disclosed herein may be improved by at least or about 10%. Plant performance as determined by a metric as disclosed herein may be improved by at least or about 30%. Plant performance as determined by a metric as disclosed herein may be improved by at least or about 50%.

Improved plant performance as determined by a metric as disclosed herein, in some instances, occurs following administration of light enriched or supplemented with UV-B. In some instances, improved plant performance as determined by a metric as disclosed herein occurs at least or about 1 day, 2 days, 3 days, 4 days, 5 days, 6 days, 7 days, 8 days, 9 days, 10 days, 12 days, 14 days, 16 days, 18 days, 20 days, 24 days, 30 days, 32 days, 50 days, 72 days, or more than 72 days following administration of light enriched or supplemented with UV-B. In some instances, improved plant performance as determined by a metric as disclosed herein occurs at least or about 1 week, 2 weeks, 3 weeks, 4 weeks, 5 weeks, 6 weeks, 7 weeks, 8 weeks, 9 weeks, 10 weeks, 11 weeks, 12 weeks, or more than 12 weeks following administration of light enriched or supplemented with UV-B.

Improved plant performance as determined by a metric as disclosed herein may be measured by resistance to infection. Infection may be caused by organisms including, but not limited to, fungi, oomycetes, bacteria, viruses, viroids, virus-like organisms, phytoplasmas, protozoa, nematodes and parasitic plants. In some cases, following UV-B treatment of the plant as described in the methods herein, plant performance is unaffected or improved despite infections caused by such organisms. In some cases, following UV-B treatment of the plant as described in the methods herein, plant performance is improved despite infections caused by such organisms as compared to non-UV-B irradiated plant. In some cases, plant performance is inspected for infections caused by such organisms. Often plant performance is inspected for at least one of leaf disease, ear rot disease, stalk rot disease, and seeding and root disease.

In some instances, improved plant performance as determined by a metric as disclosed herein is measured by at least one of a reduction in fertilizer, herbicide, insecticide, and pesticide use without affecting plant performance. Reduction to fertilizer, herbicide, insecticide, or pesticide use may be determined by comparison to the industry use for a crop over ten years, to the state-wide average, or the national average. The reduction of fertilizer, use may be at least 5%. In some cases, the reduction of fertilizer is in the range of about 5%-100%, 10%-90%, 20%-80%, 30%-70%, 40%-60%, 50%-95%, 65%-85%, or 75%-95%. In some instances, the reduction of herbicide use is at least 5%. In some cases, the reduction of herbicide is in the range of about 5%-100%, 10%-90%, 20%-80%, 30%-70%, 40%-60%, 50%-95%, 65%-85%, or 75%-95%. In some instances, the reduction of insecticide use is at least 5%. In some cases, the reduction of insecticide is in the range of about 5%-100%, 10%-90%, 20%-80%, 30%-70%, 40%-60%, 50%-95%, 65%-85%, or 75%-95%. In some instances, the reduction of pesticide use is at least 5%. In some cases, the reduction of pesticide is in the range of about 5%-100%, 10%-90%, 20%-80%, 30%-70%, 40%-60%, 50%-95%, 65%-85%, or 75%-95%.

Accordingly, UV-B supplementation or enrichment enables methods of growing crops such that pesticide use, herbicide use, fertilizer administration, or water administration may be reduced relative to untreated plants without any concomitant decrease in plant performance. In some cases, UV-B supplementation enables a substantial decrease in overall environmental impact without decrease in crop plant performance.

Improved plant performance as determined by a metric as disclosed herein may be determined by comparison of UV-B irradiated plants to non-UV-B irradiated plants. In some instances, improvements in plant performance is determined in the resultant crops from UV-B irradiated plants that are compared to crops grown under similar conditions but from plants are not administered UV-B using methods described herein. Similar conditions may be similar environment or similar growing conditions. Environmental factors include, but are not limited to, sun exposure, temperature, soil composition, soil moisture, wind, humidity, and soil pH. Growing conditions, include but are not limited to, amount of watering, amount of pesticide, amount of herbicide, amount of insecticide, duration of priming, duration of germination, and timing of sowing. In some instances, the resultant crops are compared to crops grown at a same time. For example, the crops grown at the same time are grown on an adjacent or nearby field. In some instances, the resultant crops are compared to crops from a previous growing season. In some instances, a plant performance of the resultant crops is compared to a comparable crop. In some instances, plant performance from a comparable crop is referred to standard plant performance. In some instances, the comparable crop is a crop that is grown at a same time or subject to similar growing conditions.

Improvements plant performance may be determined by comparison of a field comprising UV-B irradiated plants to a field comprising non-UV-B irradiated plants. In some instances, improvements in plant performance is determined in the resultant crops of a field from UV-B irradiated plants that are compared to a field comprising crops grown under similar conditions but from plants that are not administered UV-B using methods described herein. Similar conditions may be similar environment or similar growing conditions. Environmental factors include, but are not limited to, sun exposure, temperature, soil composition, soil moisture, wind, humidity, and soil pH. Growing conditions, include but are not limited to, amount of watering, amount of pesticide, amount of herbicide, amount of insecticide, duration of priming, duration of germination, and timing of sowing. In some instances, the field comprising UV-B irradiated plants is compared to the field comprising non-UV-B irradiated plants grown at a same time. The fields may be adjacent fields or nearby fields. The fields may be fields of comparable size. In some instances, the field comprising UV-B irradiated plants is compared to a field comprising non-UV-B irradiated plants from a previous growing season. In some instances, the field comprising UV-B irradiated plants is compared to a historical average of fields comprising non-UV-B irradiated plants. In some instances, the field comprising UV-B irradiated plants is compared to an expected average plant performance for a field comprising non-UV-B irradiated plants. In some instances, the expected average plant performance for a field is based on a national average. In some instances, the expected average plant performance for a field is based on a historical average for a particular growing region.

In some embodiments, a method to evaluate the benefits of the disclosure is a “Hardiness index” as described below in detail. This is an integrated method for assessing the response of plants to UV light, as related to key combined physiological changes in plants in response to the treatment. In other words, the observation of several key physiological responses which have occurred simultaneously is one indication that plants have responded to treatment in a manner which should be beneficial for long term plant growth and subsequently improved plant performance and/or quality.

It should be appreciated that plants of different crop type, variety, and growing location may require amended hardiness indices, in order to fully assess hardiness in those particular plants. Amendments to the hardiness index may include the integration of other seedling or growing environment variables as required.

### 3. Hardiness Index

Throughout this specification the term hardiness index is defined according to the calculation provided below,

$$H = \frac{{SDW}^{T}}{{SDW}^{N}} + \frac{{SSLW}^{T}}{{SSLW}^{N}} + \frac{1 / {SLA}^{T}}{1 / {SLA}^{N}}$$

wherein:
H=Hardiness
SDW=Shoot dry weight
SSLW=Shoot specific leaf weight
SLA=Shoot leaf area
T=Treated plants; and
N=Non treated plants.

The shoot specific leaf weight (SSLW) defines the ratio of the dry weight of the leaf per unit leaf area, whereas the term shoot leaf area (SLA) simply defines the leaf area.

Furthermore, it should be appreciated that the use of the “1/SLA” function may be merely to provide a positive H value for ease of reference, and is not essential to the disclosure.

Without this 1/SLA function, the H value may be more difficult (but not impossible) to comprehend in certain circumstances. This is because the H value may, in some cases, decrease with improved hardiness. This result may arise when the plant's shoot leaf area (SLA) increases as a result of UV exposure according to the present disclosure. This increase in SLA may be seen as an improvement to hardiness in some plant varieties.

Yet, in other plant varieties, UV treatment may lead to an increase in SLA, which may actually increase hardiness in that variety. In such a case, it may be beneficial to adapt the Hardiness index as shown below, such that the SLA is not 1/SLA.

$$H = \frac{{SDW}^{T}}{{SDW}^{N}} + \frac{{SSLW}^{T}}{{SSLW}^{N}} + \frac{{SLA}^{T}}{{SLA}^{N}}$$

Regardless, it is clear the hardiness index may be adapted and may be able to account for these differences in plant varieties.

For instance, plant seedlings with a H value between 3.01 to 15 could be identified as those which are displaying increased hardiness following treatment.

The lower H value of 3.01 reflects that each of the three values should display a value of over equal to or over 1, reflecting a positive change to the plant seedling as a result of UV treatment. Therefore, an H value of 15 represents a very significant improvement or prediction for plant hardiness.

A range of H values between 3.01 to 15 is considered to be beneficial because this range corresponds to overall plant characteristics that are more likely to withstand typical stresses in the outdoor environment.

Even small increases in the H value may mean comparatively large increases in relative hardiness characteristics. For example, an increase in the H value by 0.1, indicates a 10% increase in relative hardiness.

It should be appreciated that measuring the H value typically requires destruction of the plant seedling. Therefore, individual test seedlings from a batch may be used to determine a representative H value for the batch before selecting batches or individual plant seedlings from a batch.

In some instances, hardiness comprises improved resilience following at least one of heat, flood, drought, frost, unusual climate events, salinity stress, and high visible light stress. In some instances, improved resilience in plant seedling or plants UV-B irradiated comprises ability to germinate despite exposure to stress. In some instances, the plant seedling or plants are inspected following at least one of heat, flood, drought, frost, unusual climate events, salinity stress, and high visible light stress.

Hardiness in seedlings or plants irradiated with UV-B may be increased by a significant percentage when compared to counterpart seedlings or plants that have not been irradiated with a UV-B regimen disclosed herein. Hardiness may be increased by about 5%-100%, 10%-90%, 20%-80%, 30%-70%, 40%-60%, 50%-95%, 65%-85%, or 75%-95%. Hardiness may be increased by at least about 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 99%, or 100%. Hardiness may be increased by at least or about 5%. Hardiness may be increased by at least or about 10%. Hardiness may be increased by at least or about 30%. Hardiness may be increased by at least or about 50%.

Additional methods to evaluate or predict hardiness and/or plant performance of a plant at harvest include, but are not limited to, relative growth rate, or “RGR” (change in growth parameter between a first and second time point, divided by days between time points, expressed relative to original size at first time point (this is often used to measure the actual plant performance at the point of harvest), incorporation of increases in leaf phenolic chemical content; incorporation of increases in seedling photosynthetic health; and/or incorporation of reduction of seedling hypocotyl length.

In some instances, methods treatments as described herein and the use of the hardiness index and/or RGR are used to measure the beneficial outcomes in relation to hardiness and/or subsequent increased plant performance or quality. In some instances, the methodology allows mechanisms for selecting seedlings or related seedlings undergoing the same or similar UV treatment for a subsequent growth phase or using a particular UV-dosage regime for subsequent seedling treatments. For example, seedlings shown to first have an increased hardiness index often then go on to provide an increase in plant performance and quality. Alternatively, subsequent treatments may be fine-tuned depending on the RGR of preliminary trials to further improve results.

Provided herein are methods comprising using UV-B in a specific wavelength range to provide the beneficial results. In some instances, the method is seen to beneficially improve plant performance and/or quality across a wide range of plants. In some instances, the method is seen to increase seedling dry weight, increase in leaf weight or specific leaf weight and/or decreases in leaf area. In some instances, the method also appears to protect the plants against stresses including weather damage, disease and insect pest attack that may otherwise be detrimental in vulnerable plants. In some instances, the method is seen to work well with a wide variety of plants in preliminary studies.

### II. PLANTS

In certain aspects, described herein is a plant generated by a method as disclosed herein, wherein the plant comprises increased expression of an Oleosin (OLE), or diacylglycerol acyltransferase (DGAT) gene compared to a plant not generated by a method as disclosed herein.

A plant comprising an increased concentration of at least one lipid, wherein the plant does not comprise a transgenic gene.

A plant comprising an increased concentration of at least one lipid, wherein the plant comprises increased expression of an Oleosin (OLE) or diacylglycerol acyltransferase (DGAT) gene.

Application to the type of plants envisioned is previously described above. In some embodiments, a plant comprises increased or decreased expression of a gene as described herein. In some embodiments, the expression of the gene is not increased or decreased by a transgenic method or a transgenic gene.

### III. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

As used herein, the term “about” a number refers to that number plus or minus 10% of that number. The term “about” a range refers to that range minus 10% of its lowest value and plus 10% of its greatest value.

Throughout the specification the phrase “prior to a subsequent growth phase” should be taken as meaning either prior to the plant seedling being transferred into an outdoor environment, or in some cases being retained indoors, at a particular time point based on the age, size other feature of the plant seedling or environmental characteristics. The growth phase of the plant is typically the phase when the plant exhibits substantial growth and development into a mature plant prior to harvesting.

Throughout this specification the term “hardiness” should be taken as meaning the ability of a plant to withstand or help protect against one or more stresses during crop production and which may allow for more desirable plant performance and/or quality of the plant at harvesting.

Throughout this specification the term “plant seedling” should be taken as meaning a young plant following germination from a seed. The plant seedling may be of a vegetable, fruit, tree, shrub, herb, grass origin, and so forth.

Throughout this specification the term “plant” should be taken as meaning a matured plant seedling which is ultimately used for crops or other applications.

Although the present disclosure has particular application to vegetable and fruit crop production, it is also possible the disclosure may be used to improve other types of plant hardiness such as trees, grasses, flowers, herbs and so forth. For simplicity, the remainder of the specification will refer to crop production (and particularly vegetables), although it should be appreciated this is not intended to be limiting.

Throughout this specification the term “crop” should be taken as meaning a cultivated plant which is harvested typically by a human or machine at some point during its growth stage for further use or human consumption. However, it should be appreciated that application of the methods to grasses, trees and so forth, may be used merely to improve the hardiness without any intention to harvest.

Throughout this specification the term “indoors” should be taken as meaning a housing, typically a greenhouse, plastic polytunnel, a shade cloth with no walls, or fully indoor system which might use artificial lighting.

In some embodiments, harvesting a plant comprises grazing an animal on the plant.

In the example of a greenhouse, it may include transparent walls and/or ceiling to allow natural light in. The indoor housing may be used to allow the initial germination and seedling development phase to occur and is used during the UV irradiation exposure of the present disclosure prior to a subsequent growth phase in an outdoor environment.

In some embodiments, the treatment of the plant seedlings or plant material occurs indoors. For example, any one of the methods may occur indoors, for example, a greenhouse. In some instances, UV-B is administered indoors. In some instances, UV-B is administered outdoors. In some instances, UV-B is administered outdoors in a field.

In some instances, an advantage of conducting the treatment indoors is that it may help to regulate the conditions whilst the plant seedling is particularly vulnerable. Additionally, it may mean that the device used to apply the UV treatment may be better protected and secured. However, it is possible the treatment of the present disclosure may also be conducted in an outdoor environment, depending on the circumstances and type of seedlings or plant material to be treated.

Throughout this specification the term “transplantation” should be taken as meaning the act of transferring the plant seedling into an outdoor environment such as a field to allow continued growth prior to ultimate harvesting of the crops. The term transplantation shock refers specifically to the stress or shock incurred by the plant at the time of transplantation, for instance due to sun shock due to the different sun exposure seen between indoors and the outdoor environment.

The term “seed for sowing” as used herein refers to any embryonic plant prior to, and/or intended to be used for, planting to grow any form of plant life or crop for subsequent use (typically, but not solely, for human and animal consumption). Substantially any type of seed may be used α-cording to the present disclosure, of which there are currently about 35,000 types currently known worldwide, as noted in U.S. Pat. No. 8,001,722. Results indicated that UV-B treatment of seeds improved plant performance that is extendable to any plant species.

Some non-limiting examples of seeds for are seeds of agricultural or ornamental plants, such as lettuce, beans, broccoli, cabbage, carrot, cauliflower, cucumber, melon, onion, peas, peppers, pumpkin, spinach, squash, sweetcorn, tomato, watermelon, alfalfa, canola, corn, cotton, sorghum, soybeans, sugarbeets, wheat, mint, sunflowers, or other agriculturally or ornamentally relevant plant species.

The term “seed” refers to an embryonic plant enclosed in a protective outer covering. The formation of the seed is part of the process of reproduction in seed plants, the spermatophytes, including gymnosperm and angiosperm plants. Seeds are the product of the ripened ovule, after fertilization by pollen and some growth within the mother plant. The embryo is developed from the zygote and the seed coat from the integuments of the ovule.

The term “seed germination” refers to a process by which a seed embryo develops into a seedling. It involves the activation of the metabolic pathways that lead to growth, and the emergence of the radicle or seed root and plumule or shoot. In general, seed germination is initiated through stratification, which varies among plant species according to their original ecological setting. Of-ten though not uniformly, seed germination is triggered through a three-phase process involving water imbibition, lag phase, and radicle emergence. Seed germination may be affected by environmental conditions including, but not limited to, water, oxygen, temperature, and light.

In some embodiments, the term “plant performance” refers to a measurable metric of a plant, with certain non-limiting examples disclosed herein.

The term “long-term hardiness” or “hardiness” as used herein refers to the ability of a plant to withstand one or more stresses during crop production and to allow desirable plant performance and/or quality of the plant at harvesting. Some non-limiting examples of how improved plant performance is measured include weight of harvestable crop material, such as lettuce leaves, soybeans, tomato fruit, in com-parison to harvestable crop material where the seeds for sowing were not treated with UV-B. Other examples of how improved plant performance are measured include fresh shoot weight or whole plant dry weight, improved germination of seeds resulting from the treatment method, and improved water use efficiency of the resulting plant. In some cases, improved quality is assessed as a quantitative or qualitative assessment of at least one of a lack of blemishes on the crop (either internal or on the surface, typically from insects), improved shelf life, improved resistance to bruising or other post-harvest handling, lack of deformities, lack of irregular shapes, lack of irregular sizes, improved taste, size, shape, color, and texture. An advantage of the present disclosure is that both stress resilience and plant performance were observed (often these traits can work in an inverse relationship, where resilience is achieved at the cost of plant performance as seen with UV-C treatment).

The term “ultraviolet (UV) irradiation” as used herein refers to electromagnetic radiation with a wavelength shorter than visible light, but longer than X-rays, and is in between the range of 10 nm to 400 nm (corresponding to 3 eV to 124 eV). The UV radiation spectrum is considered to be invisible to humans, and therefore differentiated from visible light in the spectrum of about 400 nm to 700 nm.

The term “UV-B radiation” as used herein refers to radiation specifically within the waveband of 320 nm to 280 nm (herein described as the UV-B range). This is distinguishable from the UV-C waveband (280 to 100 nm) and UV-A waveband (400-320 nm). It should also be distinguishable from natural sunlight which although provides UV-B radiation, also includes other UV radiation. In some cases, the UV-B radiation is administered via LED lights.

The term “harvestable crop material” as used herein refers to any material from the plant which may be harvested to be used for subsequent purposes or human or animal consumption. Often the crop material is harvested seeds to be consumed as food or used for subsequent planting or breeding purposes. The harvested material includes but is not limited to a fruit, a vegetable, a tree, a shrub, a grass, an herb, and an extract or component of any one of the above crop materials. In some cases, the present disclosure is the material that is actually harvested, or the material used to build plant performance without any harvesting. A non-limiting example of material not in-tended to be farmed is forest regeneration. Some non-limiting examples of harvestable crop mate-rial are lettuce, beans, broccoli, cabbage, carrot, cauliflower, cucumber, melon, onion, peas, pep-pers, pumpkin, spinach, squash, sweetcorn, tomato, watermelon, alfalfa, canola, corn, cotton, sorghum, soybeans, sugarbeets, wheat and combinations thereof.

A “fruit” refers strictly to any seed-containing organ of a plant. More informally, the term in some cases refers to harvestable material generally.

The term “flavonoid” as used herein refers to a class of plant secondary metabolites which have the general structure of a 15-carbon skeleton, consisting of two phenyl rings and heterocyclic ring (C6-C3-C6). Flavonoids are associated in some cases with stress resistance, such that an increase in their accumulation levels corresponds to an increase in plant stress resistance.

### IV. LIST OF ENUMERATED EMBODIMENTS

1. A method of increasing the total lipid percent in a grass by administering UV-B light to the seed prior to sowing.
2. The method of embodiment 1, wherein the grass comprises a member of the Poaceae family.
3. The method of embodiment 1 or 2, wherein the grass comprises a forage grass.
4. The method of embodiment 2, wherein the grass comprises a ryegrass or a bluegrass.
5. The method of embodiment 4, wherein the grass comprises ryegrass.
6. The method of any one of embodiments 1-5, wherein the lipid comprises a triacylglycerol.
7. The method of any one of embodiments 1-5, wherein the lipid comprises a fatty acid.
8. The method of embodiment 7, wherein the fatty acid comprises palmitic acid, palmitoleic acid, stearic acid, oleic acid, linoleic acid, linolenic acid, arachidic acid, gondoic acid, behenic acid, or erucic acid.
9. The method of any one of embodiments 1-8, wherein the total lipid percent is measured by gas chromatography.
10. A method of increasing the rate of photosynthesis in a plant, the method comprising: administering a light treatment comprising UV-B to a seed and increasing the rate of photosynthesis, wherein the increased net rate of photosynthesis is measured by infrared gas analysis.
11. The method of embodiment 10, wherein the rate of photosynthesis is increased in a plant compared to a plant wherein the seed was not administered UV-B light prior to sowing.
12. The method of embodiment 10 or 11, wherein the rate of photosynthesis is increased by at least 1%, 2%, 3%, 4%, 5%, 6%, 7%, 8%, 9%, 10% or more.
13. A method of improving a field for grazing, the method comprising sowing a plurality of ryegrass seeds in the field, wherein the plurality of ryegrass seeds were administered UV-B light prior to sowing.
14. The method of embodiment 13, wherein the field is improved compared to a field wherein the plurality of ryegrass seeds was not administered UV-B light prior to sowing.
15. The method of embodiment 13 or 14, wherein the field is improved by at least 1%, 2%, 3%, 4%, 5%, 6%, 7%, 8%, 9%, 10% or more.
16. The method of any one of embodiments 13-15, wherein improved comprises an increase in the lipid content of the ryegrass grown in the field.
17. The method of any one of embodiments 13-16, wherein improved comprises decreasing the area of land required for grazing animals.
18. A method of increasing the lipid content in a field for grazing, the method comprising sowing a plurality of ryegrass seeds in the field, wherein the plurality of ryegrass seeds were administered UV-B light prior to sowing.
19. The method of embodiment 18, wherein the lipid content in the field is improved compared to a field wherein the plurality of ryegrass seeds was not administered UV-B light prior to sowing.
20. The method of embodiment 18 or 19, wherein the lipid content in the field is improved by at least 1%, 2%, 3%, 4%, 5%, 6%, 7%, 8%, 9%, 10% or more.
21. A method of decreasing the area of land required for grazing animals, the method comprising sowing a plurality of ryegrass seeds in the field, wherein the plurality of ryegrass seeds were administered UV-B light prior to sowing.
22. A method of increasing the lipid content of grazing animals, the method comprising 1) administering a light treatment comprising UV-B to a plurality of ryegrass seeds, 2) sowing the plurality of ryegrass seeds in a field, 3) grazing the animals on the field.
23. A method of increasing the lipid content of grazing animals, the method comprising 1) administering a light treatment comprising UV-B to a plurality of soybean seeds, 2) sowing the plurality of soybean seeds in a field, 3) feeding the resultant plants to the animals.
24. A method of reducing the methane production of grazing animals, the method comprising 1) administering a light treatment comprising UV-B to a plurality of grass seeds, 2) sowing the plurality of grass seeds in a field, 3) grazing the animals on the field.

25. The method of embodiment 24, wherein the grass is a ryegrass.
26. The method of embodiment 24 or 25, wherein methane production is reduced by at least about 1%, 2%, 3%, 4%, 5%, 6%, 7%, 8%, 9%, 10%, 11%, 12%, 13%, 14%, 15%, 16%, 17%, 18%, 19% or 20% as compared to a grazing animal grazed on a field not sown with grass seeds administered UV-B.
27. The method of any one of embodiments 21-26, wherein the grazing animal is a cow or a sheep.
28. A method of sequestering carbon from the atmosphere using a non-transgenic plant, the method comprising: 1) administering a light treatment comprising UV-B to a plurality of seeds, and 2) sowing the plurality of seeds in a field.
29. The method of any one of embodiments 21-28, wherein the resultant plants from the plurality of seeds have an elevated lipid level.
30. The method of any one of embodiments 1-29, wherein the treatment is administered to a seed prior to sowing.
31. The method of any one of embodiments 1-30, wherein the UV-B light comprises UV-B light with a waveband of about 280-290 nm.
32. The method of any one of embodiments 1-31, wherein the UV-B light does not comprise UV-A or UV-C.
33. A plant comprising an elevated concentration of at least one lipid, wherein the plant does not comprise a transgenic gene.
34. The plant of embodiment 33, wherein the transgenic gene is OLE or DGAT.
35. The plant of embodiment 33 or 34, wherein the elevated concentration is at least a relative increase of 3% total plant lipids per unit of plant dry matter compared to a plant that has not been administered UV-B light.
36. The plant of any one of embodiments 33-35, wherein the elevated concentration is at least 0.5% of total plant lipid concentration per unit of plant dry matter.
37. The plant of any one of embodiments 33-36, wherein the plant comprises a grass.
38. The plant of embodiment 37, wherein the grass comprises a member of the Poaceae family.
39. The plant of embodiment 37, wherein the grass comprises a forage grass.
40. The plant of embodiment 37, wherein the grass comprises a ryegrass or a bluegrass
41. The plant of embodiment 40, wherein the grass comprises ryegrass.
42. The plant of any one of embodiments 33-36, wherein the plant is a soybean.
43. The plant of any one of embodiments 33-42, wherein the lipid comprises a triacylglycerol.
44. The plant of any one of embodiments 33-43, wherein the lipid comprises a fatty acid.
45. The plant of embodiment 39-44 wherein the fatty acid comprises palmitic acid, palmitoleic acid, stearic acid, oleic acid, linoleic acid, linolenic acid, arachidic acid, gondoic acid, behenic acid, or erucic acid.
46. The plant of embodiment 33, wherein the plant is an annual ryegrass, wherein the elevated concentration of the lipid comprises at least 4% dry mass.
47. The plant of any one of embodiments 33-46, wherein the plant has been administered UV-B light.
48. The plant of embodiment 47, wherein the UV-B light was administered to a seed prior to sowing.
49. The plant of embodiment 47 or 48, wherein the UV-B light comprises UV-B light with a waveband of about 280-290 nm.
50. The plant of any one of embodiments 47-49, wherein light in the wavebands of UV-A or UV-C was not administered to the plant.

### V. EXAMPLES

The following examples are included for illustrative purposes only and are not intended to limit the scope of the invention.

### Example 1: Treating Ryegrass Seeds with UV-B-Forage Quality, Forage Biomass, and Tiller Number

Ryegrass seeds were treated with UV-B light and sown into pots. The pots were randomized through a controlled environment room (Conditions: 12-16 h photoperiod, Photosynthetically Active Radiation 300-400 μmol m⁻²). 15-30 plants were included in each treatment group. Tiller number, shoot biomass from grazing simulation and forage quality were measured. Total plant lipids, an indicator of forage quality, increased by up to 17% at 59 days after sowing (FIG. 1). Each treatment group contained 10 plants. Simulated forage biomass increased by up to 37% by 20 days after sowing (FIG. 2). Each treatment group contained 15 plants. All tillers were cut and weighed at 5 cm from the soil. Tiller number increased by 21% by 37 days after sowing (FIG. 3). Each treatment group contained 30 plants.

### Example 2: Treating Ryegrass Seeds with UV-B—Non-Structural Carbohydrate Concentration and Metabolizable Energy

Ryegrass seeds were treated with UV-B light and sown into pots. The pots were randomized through a controlled environment plant growth room (Conditions: 12-16 h photoperiod, Photosynthetically Active Radiation: 300-400 μmol m⁻²) with 64 pots per treatment group. Non-structural carbohydrates and metabolizable energy were measured.

36 days after sowing, plant tissue was harvested from the plants and pooled into replicate samples, which were then analyzed for non-structural carbohydrate concentration, as a percentage of total plant tissue dry matter. Soluble or ‘free’ sugars in plant tissue were analyzed by extraction with aqueous ethanol, followed by determination using a phenol-sulphuric acid colorimetric procedure. Non-structural carbohydrate concentration was increased by 84% in the UV-B treatment group compared to controls. The results of the non-structural carbohydrate concentration measurements are shown in Table 1.

TABLE 1
Non-Structural Carbohydrate Concentration

| | Mean | | | | | |
|---|---|---|---|---|---|---|
| | (NSC as % | | | | % increase | |
| | of plant | St | Sample | St | vs. | |
| Treatment | Dry Matter) | dev | n | error | Control | P-value |
| Control | 3.23 | 2.39 | 4 | 1.19 | — | — |
| UV-B seed | 5.93 | 1 | 3 | 0.58 | 84% | 0.1 |
| treatment | | | | | | |

After sowing and a growth period, plant tissue was harvested from the plants and pooled into replicate samples, which were then analyzed for metabolizable energy concentration, as a relative amount of total plant tissue dry matter. Metabolizable Energy (ME) analysis was based on a pepsin-cellulase digestion method to give an Organic Matter Digestibility (OMD %) measure. The in-vitro OMD % result obtained from this method was adjusted to predict an in-vivo OMD % value using a linear regression that is based on a series of samples with documented in-vivo results. Digestibility results were reported on a dry weight basis as DOMD %. ME is then derived from the DOMD % of feed samples by published calculation and is also reported on a dry weight basis. The universal equation of ME=DOMD %×0.16 was used. The results of the ME analysis measurements are shown in Table 2.

TABLE 2
Metabolizable Energy

| | | | | | % increase | |
|---|---|---|---|---|---|---|
| | Mean (MJ/Kg | St | Sample | St | vs. | |
| Treatment | Dry Matter) | dev | n | error | Control | P-value |
| Control | 12.28 | 0.12 | 6 | 0.05 | | |
| UV-B | 12.52 | 0.17 | 6 | 0.07 | 1.9% | 0.02 |
| seed | | | | | | |
| treatment | | | | | | |

### Example 3: Treating Corn Seeds with UV-B Metabolizable Energy and Lipid Concentration

Corn seeds were treated with UV-B light and sown into two consecutive year field trials. The field trials employed a randomized and replicated plot design for each treatment/Control group. Each plot was 10 meters long×4 rows. For metabolizable energy analysis in corn sileage, 6 replicated plots per treatment/Control group were sown. For crude fat concentration in corn sileage 4 replicated plots per treatment/Control group were sown. At final maturity stage for corn sileage production, cob and stalk samples from each plot were hand-harvested, weighed, and homogenized. Randomly selected sub-samples were analyzed for crude fat (also referred to as plant lipids) concentration, and metabolizable energy concentration, both on a per amount of sample dry matter basis. Crude fat was determined using NIR spectroscopy analysis.

Metabolizable Energy analysis was based on a pepsin-cellulase digestion method to give an Organic Matter Digestibility (OMD %) measure. The in-vitro OMD % result obtained from this method is adjusted to predict an in-vivo OMD % value using a linear regression that is based on a series of samples with documented in-vivo results. Digestibility results were reported on a dry weight basis as DOMD %. ME is then derived from the DOMD % of feed samples by published calculation and is also reported on a dry weight basis. The universal equation of ME=DOMD %×0.16 was used. Metabolizable energy concentration in corn sileage increased by 5% (FIG. 4). Crude fat concentration in corn sileage increased by 9.4% (FIG. 5).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of increasing the performance of a plant, the method comprising: 1) administering UV-B light to a plant seed, 2) sowing the plant seed, 3) growing the plant from the plant seed, and 4) harvesting the plant, wherein the performance is measured by total lipid percentage, metabolizable energy, non-structural carbohydrates, crude fat concentration, forage biomass, tiller number, or shoot fresh weight.

2. A method of increasing total lipid percentage in a plant, the method comprising: 1) administering UV-B light to a plant seed, 2) sowing the plant seed, 3) growing the plant from the plant seed, and 4) harvesting the plant.

3. A method of increasing the total lipid percentage in a field for grazing, the method comprising: 1) administering UV-B light to a plurality of plant seeds, 2) sowing the plurality of plant seeds in the field, and 3) growing plants from the plurality of plant seeds.

4. The method of any one of claims 1-3, wherein the lipid percentage is increased in plants grown from plant seeds administered UV-B light prior to sowing as compared to plants grown from plant seeds not administered UV-B light prior to sowing.

5. The method of claim 1-4, wherein the lipid percentage is increased by about 1% to about 100%.

6. The method of claim 5, wherein the lipid percentage is increased by about 1% to about 25%.

7. The method of claim 5, wherein the lipid percentage is increased by about 5% to about 20%.

8. The method of any one of claims 1-4, wherein the lipid percentage is increased by about 1% to about 100% total plant lipids per unit of plant dry matter.

9. The method of claim 8, wherein the lipid percentage is increased by about 1% to about 25% total plant lipids per unit of plant dry matter.

10. The method of any one of claims 1-4, wherein the lipid comprises a triacylglycerol, phospholipid, sterol, galactolipid, fatty acid, or tocopherol.

11. The method of claim 10, wherein the phospholipid comprises phosphatidylcholine or phosphatidylethanolamine.

12. The method of claim 10, wherein the sterol comprises sitosterol, campesterol, or stigmasterol.

13. The method of claim 10, wherein the galactolipid comprises digalactosyldiacylglycerol or monogalactosyldiacylglycerol.

14. The method of claim 10, wherein the tocopherol comprises Vitamin E.

15. The method of claim 10, wherein the fatty acid comprises palmitic acid, palmitoleic acid, stearic acid, oleic acid, linoleic acid, linolenic acid, arachidic acid, gondoic acid, behenic acid, or erucic acid.

16. The method of any one of claims 1-15, wherein the total lipid percent is measured by gas chromatography or near infrared spectroscopy.

17. A method of increasing metabolizable energy in a plant, the method comprising: 1) administering UV-B light to a plant seed, 2) sowing the plant seed, 3) growing the plant from the plant seed, and 4) harvesting the plant.

18. The method of claim 17, wherein metabolizable energy is increased in plants grown from plant seeds administered UV-B light prior to sowing as compared to plants grown from plant seeds not administered UV-B light prior to sowing.

19. The method of claim 18, wherein metabolizable energy is increased by about 1% to about 100%.

20. The method of claim 19, wherein metabolizable energy is increased by about 1% to about 15%.

21. The method of claim 18, wherein metabolizable energy is increased by about 0.1 to about 10 MJ/Kg of dry matter.

22. The method of claim 21, wherein metabolizable energy is increased by about 0.1 to about 0.5 MJ/Kg of dry matter.

23. The method of claim 22, wherein metabolizable energy is increased by about 1 to about 5 MJ/Kg of dry matter.

24. A method of increasing non-structural carbohydrates in a plant, the method comprising: 1) administering UV-B light to a plant seed, 2) sowing the plant seed, 3) growing the plant from the plant seed, and 4) harvesting the plant.

25. The method of claim 24, wherein non-structural carbohydrates are increased in plants grown from plant seeds administered UV-B light prior to sowing as compared to plants grown from plant seeds not administered UV-B light prior to sowing.

26. The method of claim 25, wherein non-structural carbohydrates are increased by about 1% to about 100%.

27. The method of claim 26, wherein non-structural carbohydrates are increased by about 1% to about 15%.

28. The method of claim 27, wherein non-structural carbohydrates are increased by about 1% to about 10% of plant dry matter percentage.

29. The method of claim 28, wherein non-structural carbohydrates are increased by about 1% to about 5% of plant dry matter percentage.

30. A method of increasing crude fat concentration in a plant, the method comprising: 1) administering UV-B light to a plant seed, 2) sowing the plant seed, 3) growing the plant from the plant seed, and 4) harvesting the plant.

31. The method of claim 30, wherein crude fat concentration is increased in plants grown from plant seeds administered UV-B light prior to sowing as compared to plants grown from plant seeds not administered UV-B light prior to sowing.

32. The method of claim 31, wherein crude fat concentration is increased by about 1% to about 100%.

33. The method of claim 32, wherein crude fat concentration is increased by about 1% to about 15%.

34. The method of claim 33, wherein crude fat concentration is increased by about 1% to about 100% of plant dry matter percentage.

35. The method of claim 34, wherein crude fat concentration is increased by about 1% to about 15% of plant dry matter percentage.

36. A method of increasing forage biomass in a grass, the method comprising: 1) administering UV-B light to a plant seed, 2) sowing the plant seed, 3) growing the plant from the plant seed, and 4) harvesting the plant.

37. The method of claim 36, wherein forage biomass is increased in plants grown from plant seeds administered UV-B light prior to sowing as compared to plants grown from plant seeds not administered UV-B light prior to sowing.

38. The method of claim 37, wherein forage biomass is increased by about 1% to about 100%.

39. The method of claim 38, wherein forage biomass is increased by about 1% to about 15%.

40. A method of increasing tiller number in a grass, the method comprising: 1) administering UV-B light to a plant seed, 2) sowing the plant seed, 3) growing the plant from the plant seed, and 4) harvesting the plant.

41. The method of claim 40, wherein tiller number of the grass is increased in plants grown from plant seeds administered UV-B light prior to sowing as compared to plants grown from plant seeds not administered UV-B light prior to sowing.

42. The method of claim 41, wherein tiller number is increased by about 1% to about 100%.

43. The method of claim 42, wherein tiller number is increased by about 1% to about 15%.

44. A method of increasing fresh weight in a grass, the method comprising: 1) administering UV-B light to a plant seed, 2) sowing the plant seed, 3) growing the plant from the plant seed, and 4) harvesting the plant.

45. The method of claim 44, wherein shoot fresh weight of the grass is increased in plants grown from plant seeds administered UV-B light prior to sowing as compared to plants grown from plant seeds not administered UV-B light prior to sowing.

46. The method of claim 45, wherein shoot fresh weight of the grass increased by about 1% to about 100%.

47. The method of claim 46, wherein shoot fresh weight of the grass increased by about 1% to about 15%.

48. The method of claim 45, wherein shoot fresh weight of the grass increased by about 1 gram to about 100 grams.

49. The method of claim 48, wherein shoot fresh weight of the grass increased by about 1 gram to about 10 grams.

50. A method of increasing the total energy content per unit of feed consumed by an animal, the method comprising 1) administering UV-B light to a plurality of plant seeds, 2) sowing the plurality of plant seeds in a field, 3) growing plant from the plurality of plant seeds, and 4) feeding the animals the feed.

51. The method of claim 50, wherein the total energy content per unit of feed consumed is increased by about 1% to about 100%.

52. The method of claim 51, wherein the total energy content per unit of feed consumed is increased by about 1% to about 25%.

53. The method of claim 51, wherein the total energy content per unit of feed consumed is increased by about 5% to about 20%.

54. A method of decreasing an area of field required for feeding animals, the method comprising: 1) administering UV-B light to a plant seed, 2) sowing the plant seed, 3) growing plant from the plant seed, and 4) grazing the animals on the field.

55. The method of claim 54, wherein the area of the field required for feeding animals is decreased compared to a field wherein the plant seed was not administered UV-B light prior to sowing.

56. The method of claim 55, wherein the area is decreased by about 1% to about 100%.

57. The method of claim 56, wherein the area is decreased by about 1% to about 15%.

58. The method of claim 55, wherein the area is decreased by about 0.1 to about 1000 acres.

59. The method of claim 55, wherein the area is decreased by about 0.1 to about 1000 hectares.

60. A method of reducing the methane production of animals, the method comprising 1) administering UV-B light to a plurality of plant seeds, 2) sowing the plurality of plant seeds in a field, 3) growing plants from the plant seeds, and 4) grazing the animals on the field or feeding the plants to the animals.

61. The method of claim 60, wherein methane production is decreased in animals fed plants grown from plant seeds administered UV-B light prior to sowing as compared to plants grown from plant seeds not administered UV-B light prior to sowing.

62. The method of claim 61, wherein methane production is decreased by about 1% to about 100%.

63. The method of claim 62, wherein methane production is decreased by about 1% to about 15%.

64. The method of any one of claims 50-63 wherein the animal is a cow or a sheep.

65. A method of sequestering carbon from the atmosphere, the method comprising: 1) administering UV-B light to a plant seed, 2) sowing the plant seed, and 3) growing the plant from the plant seed.

66. The method of claim 65, wherein sequestered carbon is increased in plants grown from plant seeds administered UV-B light prior to sowing as compared to plants grown from plant seeds not administered UV-B light prior to sowing.

67. The method of claim 66, wherein sequestered carbon is increased by about 1% to about 100%.

68. The method of claim 67, wherein sequestered carbon is increased by about 1% to about 15%.

69. The method of any one of claims 1-68, wherein the plant comprises a member of the Poaceae or Fabaceae family.

70. The method of claim 69, wherein the member of the Poaceae family comprises rice, corn, or a grass.

71. The method of claim 70, wherein the grass comprises a forage grass.

72. The method of claim 70, wherein the grass comprises perennial ryegrass (Lolium perenne), timothy grass (Phleum pratense), tall fescue (Festuca arundinacea), kentucky bluegrass (Poa pratensis), bermuda grass (Cynodon dactylon), alfalfa grass (Medicago sativa), orchard grass (Dactylis glomerata), rye grass (lolium), brome grass (bromus), sudan grass (Sorghum×drummondii), bahia grass (Paspalum notatum), clover (trifolium), meadow fescue (Festuca pratensis), switchgrass (Panicum virgatum), prairie grass (Bromus kalmii), festulolium (hybrid of ryegrass and fescue), signal grass (Urochloa panicoides), Italian ryegrass (Lolium multiflorum), reed canary grass (Phalaris arundinacea), or millet (Panicum miliaceum or Setaria italica).

73. The method of claim 72, wherein the grass comprises a ryegrass or a bluegrass.

74. The method of claim 73, wherein the grass comprises ryegrass.

75. The method of claim 74, wherein the ryegrass comprises annual ryegrass.

76. The method of claim 69, wherein the member of the Fabaceae family comprises alfalfa, clover, soybean, peas, lentils, vetch, peanut, cowpea, chickpea or, lupins.

77. The method of claim 76, wherein the member of the Fabaceae family comprises soybeans.

78. The method of any one of claims 1-77, wherein the UV-B light comprises UV-B light with a waveband of about 280-310 nm.

79. The method of claim 78, wherein the UV-B light comprises UV-B light with a waveband of about 280-290 nm.

80. The method of any one of claims 1-79, wherein the UV-B light does not comprise UV-A or UV-C.

81. A plant generated by the method of any one of claims 1-80, wherein the plant comprises increased expression of an Oleosin (OLE) or diacylglycerol acyltransferase (DGAT) gene compared to a plant not generated by the method of any one of claims 1-80.

82. The plant of claim 81, wherein the plant comprises an increased concentration of at least one lipid.

83. The plant of claim 81, wherein the expression of OLE or DGAT is not transgenically increased.

84. A plant comprising an increased concentration of at least one lipid, wherein the plant does not comprise a transgenic gene.

85. The plant of claim 84, wherein the transgenic gene is Oleosin (OLE) or diacylglycerol acyltransferase (DGAT).

86. A plant comprising an increased concentration of at least one lipid, wherein the plant comprises increased expression of an Oleosin (OLE) or diacylglycerol acyltransferase (DGAT) gene.

87. The plant of claim 86, wherein the expression of OLE or DGAT is not transgenically increased.

88. The plant of any one of claims 84-87, wherein the plant is grown from a plant seed that has been administered UV-B light.

89. The plant of any one of claims 81-88, wherein lipid concentration is increased by about 1% to about 100%.

90. The plant of claim 89, wherein lipid concentration is increased by about 1% to about 15%.

91. The plant of any one of claims 81-88, wherein lipid concentration is increased by about 0.1% to about 100% per unit of plant dry matter compared to a plant that is grown from a seed not administered UV-B light.

92. The plant of claim 91, wherein the lipid concentration is increased by about 0.1% to about 1% per unit of plant dry matter compared to a plant that is grown from a seed not administered UV-B light.

93. The plant of any one of claims 81-92, wherein the plant comprises a member of the Poaceae or Fabaceae family.

94. The plant of claim 93, wherein the member of the Poaceae family comprises rice, corn, or a grass.

95. The plant of claim 94, wherein the grass comprises a forage grass.

96. The pant of claim 94, wherein the grass comprises perennial ryegrass (Lolium perenne), timothy grass (Phleum pratense), tall fescue (Festuca arundinacea), kentucky bluegrass (Poa pratensis), bermuda grass (Cynodon dactylon), alfalfa grass (Medicago sativa), orchard grass (Dactylis glomerata), rye grass (lolium), brome grass (bromus), sudan grass (Sorghum×drummondii), bahia grass (Paspalum notatum), clover (trifolium), meadow fescue (Festuca pratensis), switchgrass (Panicum virgatum), prairie grass (Bromus kalmii), festulolium (hybrid of ryegrass and fescue), signal grass (Urochloa panicoides), Italian ryegrass (Lolium multiflorum), reed canary grass (Phalaris arundinacea), or millet (panicum miliaceum or Setaria italica).

97. The plant of claim 96, wherein the grass comprises a ryegrass or a bluegrass.

98. The plant of claim 97, wherein the grass comprises ryegrass.

99. The plant of claim 98, wherein the ryegrass comprises annual ryegrass.

100. The plant of claim 93, wherein the member of the Fabaceae family comprises alfalfa, clover, soybean, peas, lentils, vetch, peanut, cowpea, chickpea or, lupins.

101. The plant of claim 100, wherein the member of the Fabaceae family comprises soybeans.

102. The plant of any one of claims 81-101, wherein the lipid comprises a triacylglycerol, phospholipid, sterol, galactolipid, fatty acid, or tocopherol.

103. The plant of claim 102, wherein the phospholipid comprises phosphatidylcholine or phosphatidylethanolamine.

104. The plant of claim 102, wherein the sterol comprises sitosterol, campesterol, or stigmasterol.

105. The plant of claim 102, wherein the galactolipid comprises digalactosyldiacylglycerol or monogalactosyldiacylglycerol.

106. The plant of claim 102, wherein the tocopherol comprises Vitamin E.

107. The plant of claim 102, wherein the fatty acid comprises palmitic acid, palmitoleic acid, stearic acid, oleic acid, linoleic acid, linolenic acid, arachidic acid, gondoic acid, behenic acid, or erucic acid.

108. The plant of claim 98, wherein the ryegrass is an annual ryegrass, and wherein the lipid concentration is increased by about 1% to 100% total plant lipids per unit of plant dry matter.

109. The plant of claim 98, wherein the ryegrass is an annual ryegrass, and wherein the lipid concentration is increased by about 1% to 15% total plant lipids per unit of plant dry matter.

110. The plant of any one of claims 84-109, wherein the plant has been administered UV-B light.

111. The plant of claim 110, wherein the UV-B light was administered to a seed prior to sowing.

112. The plant of claim 110 or 111, wherein the UV-B light comprises UV-B light with a waveband of about 280-310 nm.

113. The plant of claim 112, wherein the UV-B light comprises UV-B light with a waveband of about 280-290 nm.

114. The plant of any one of claims 84-113, wherein light in the wavebands of UV-A or UV-C was not administered to the plant.
